(19) United States
(12) Patent Application Publication
(10) Pub. No.: US 2021/0047560 A1
(43) Pub. Date: Feb. 18, 2021

(54) HETEROCYCLIC COMPOUNDS EXHIBITING THERMALLY ACTIVATED DELAYED FLUORESCENCE (TADF) AND THEIR USE IN ELECTROLUMINESCENT DEVICES

(71) Applicant: UNIVERSITY COURT OF THE UNIVERSITY OF ST ANDREWS, St Andrews, Fife (GB)

(72) Inventors: Eli ZYSMAN-COLMAN, St. Andrews, Fife (GB); Dongyang CHEN, St. Andrews, Fife (GB); Rajamalli PACHAI GOUNDER, St. Andrews, Fife (GB)

(73) Assignee: UNIVERSITY COURT OF THE UNIVERSITY OF ST ANDREWS, St Andrews, Fife (GB)

(21) Appl. No.: 17/049,304

(22) PCT Filed: Apr. 23, 2019
(86) PCT No.: PCT/GB2019/051143

§ 371 Date: Oct. 20, 2020

(30) Foreign Application Priority Data
Apr. 20, 2018 (GB) 1806488.1

(51) Int. Cl. C09K 11/06; C07D 401/14; H01L 51/00; C07D 403/14; C07F 9/6558

(52) U.S. Cl. CPC C09K 11/06; C07D 401/14; H01L 51/0067; H01L 51/5016; C07D 403/14; C07F 9/65583; C09K 2211/1018; H01L 51/0072

(57) ABSTRACT

An organic thermally activated delayed fluorescence (TADF) compound selected from the group consisting of compounds according to formula (Ia) and formula (Ib): wherein: Het is a heteroaryl group containing at least one heteroatom; n(D) denotes n donor groups D bonding to the heteroaryl group Het; n is at least 1; and -a and -b denotes bonding to another group.

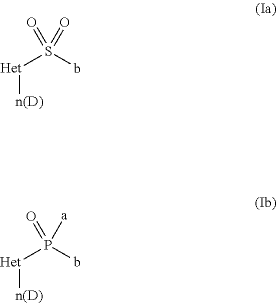

### FIELD OF THE INVENTION

The present invention is directed to the provision of Thermally Activated Delayed Fluorescence (TADF) compounds for use in electroluminescent devices such as OLEDs and Light Emitting Electrochemical Cells (LEECs).

### BACKGROUND TO THE INVENTION

Organic light-emitting diodes (OLEDs) have attracted significant attention and are now commonly used in flat panel displays such as in large-screen televisions.

The maximum internal quantum efficiency (IQE) of commercial devices is typically 25% for OLEDs using conventional fluorescent dopants, and increases to 100% for heavy metal employing phosphorescent emitters.

However, utilization of noble metals such as Ir or Pt in phosphorescent emitters may be problematic in terms of scarcity of materials and environmental concerns regarding extraction recycle and disposal.

In particular blue emitting materials present technical difficulties. Although many blue phosphorescent materials have been developed, providing acceptable device lifetimes and satisfactory depth of colour remain challenging. Alternative materials for other colours of emission are also still being sought.

Therefore, the development of new materials, highly efficient (and especially blue-emitting) to provide alternative and/or improved options is desirable.

Recently, OLEDs making use of metal-free thermally activated delayed fluorescence (TADF) emitters have arisen as a cheaper alternative to phosphorescent OLEDs.

Although a plethora of TADF emitters has been developed since 2012, only a few ‘deep-blue’ TADF OLEDs (with CIE coordinates of y<0.2 and x+y<0.30) are known.

The efficiencies of known deep blue TADF materials (and their related properties) are still generally lower than the known ‘sky blue’ and green TADF materials that have been developed.

TADF emitters have the benefit of being able to convert electrons in the lowest triplet excited state (T1) to the lowest singlet excited state (S1) via reverse intersystem crossing (RISC) using thermal energy. Thus TADF materials can harvest light from both triplet and singlet excitons.

To achieve efficient RISC, a very small singlet-triplet energy gap (ΔE_{ST}) is required. This is generally done by designing particular twisted molecular structures. However, twisted molecules tend to lead to structural relaxation phenomena, resulting in broadening and red-shifting of the emission spectra. As a result desired CIE colour coordinates (for e.g. deep blue emissions) are difficult to achieve with known TADF structures.

Therefore despite the progress made there is a need to provide improved and alternative compounds for use in display and lighting uses.

### DESCRIPTION OF THE INVENTION

According to a first aspect the present invention provides an organic thermally activated delayed fluorescence (TADF) compound selected from the group consisting of compounds according to formula Ia and formula Ib:

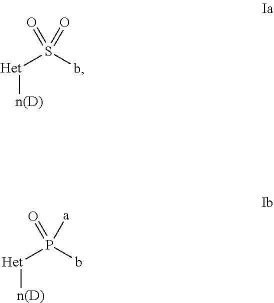

wherein:

Het is a heteroaryl group containing at least one heteroatom;

n(D) denotes n donor groups D bonding to the heteroaryl group Het;

n is at least 1; and

-a and -b denotes bonding to another group.

Thus the TADF compounds have acceptor groups of formula IIa or formula IIb:

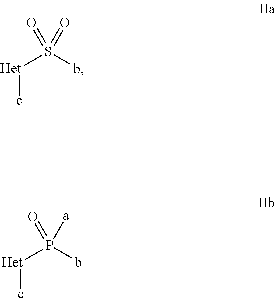

where -c denotes bonding to at least one donor group D, and -a and -b have the same meaning as in formulas Ia and Ib.

As discussed further below and with reference to some specific embodiments both sulphonate (formula IIa) and the phosphorus containing (formula IIb) acceptor groups of the invention can be used to provide TADF compounds with useful properties for OLEDs or other electroluminescent devices, for example to provide deep blue light emitting materials and corresponding devices.

The TADF compounds are typically metal free organic species. Heteroatoms may be independently selected from N, O and S. More than one heteroatom may be employed in a heteroaryl group Het.

The TADF compounds may have two heteroaryl groups Het bonded to the accepting moiety, one to each of the available bonding positions to S or P. Each Het may be the same or different. The TADF compounds may have two groups Het bonded to the accepting group and each group Het may have at least one donor group D bonded to it. Each Het may be the same or different.

As a further alternative the TADF compounds may have one group Het bonded to the accepting group and the other available bonding position (-b) may be to an aryl group (without heteroatoms in the ring or rings). The aryl group may have at least one donor group D bonded to it.

More generally the group bonded at position -b for compounds of formula Ia (sulphones) or formula Ib (phosphorus acceptors) may be selected from the group consisting of:

—H, substituted or unsubstituted primary, secondary or tertiary alkyl, that may be cyclic and may be unsaturated (for example C1-C10 or even C1-C4); substituted or unsubstituted aryl or heteroaryl; substituted or unsubstituted aryl hydroxyl; substituted or unsubstituted aryloxy; and substituted or unsubstituted thioalkyl or thioaryl. Where aryl or heteroaryl groups are present the may have donor groups D as substituents.

The group at position -a in compounds of formula Ib may be selected from the group consisting of —H, substituted or unsubstituted primary, secondary or tertiary alkyl, that may be cyclic and may be unsaturated (for example C1-C10 or even C1-C4); substituted or unsubstituted aryl or heteroaryl; substituted or unsubstituted primary, secondary or tertiary alkoxy, that may be cyclic and may be unsaturated (for example C1-C10 or even C1-C4); substituted or unsubstituted aryloxy or heteroaryloxy substituted or unsubstituted aryl hydroxyl; and substituted or unsubstituted thioalkyl or thioaryl.

Thus substituent groups at position -a may have oxygen bonding to the phosphorus. For example, substituents at position -a may be substituted or unsubstituted primary, secondary or tertiary alkoxy, that may be cyclic and may be unsaturated (for example C1-C10 or even C1-C4); substituted or unsubstituted aryloxy or heteroaryloxy and substituted or unsubstituted aryl hydroxyl. Sometimes, the substituent group at position is a substituted or unsubstituted primary, secondary or tertiary alkoxy, that may be cyclic and may be unsaturated (for example C1-C10 or even C1-C4). Often, the substituted or unsubstituted primary, secondary or tertiary alkoxy is linear. Typically, the primary, secondary or tertiary alkoxy is unsubstituted and is often ethyloxy. Where substituents bonding at position -a in compounds of formula Ib are aryl or heteroaryl, they may be substituted with one or more donor groups D.

Often, the substituent group at position -a has oxygen bonding to the phosphorus, e.g. as described above, and the phosphorus containing compound of the invention can be a phosphinate or phosphonate. In the case of a phosphinate, the group bonded at position -b of formula Ib can be selected from the group consisting of: —H, substituted or unsubstituted primary, secondary or tertiary alkyl, that may be cyclic and may be unsaturated (for example C1-C10 or even C1-C4); substituted or unsubstituted aryl or heteroaryl. Where aryl or heteroaryl groups are present they may have donor groups D as substituents. In the case of a phosphonate, the group bonded at position -b of formula Ib can be selected from the group consisting of: substituted or unsubstituted primary, secondary or tertiary alkoxy, that may be cyclic and may be unsaturated (for example C1-C10 or even C1-C4); and substituted or unsubstituted aryloxy or heteroaryloxy. Where aryl or heteroaryl groups are present they may have donor groups D as substituents.

Alternatively the substituent groups at position -a may have sulfur bonding to the phosphorus. The substituent group may be a substituted or unsubstituted thioalkyl or thioaryl. For example, the substituent group may be an R^{x} group bonded to the phosphorus atom via a sulfur atom. i.e. —S—R^{x}. R^{x} can be a substituted or unsubstituted primary, secondary or tertiary alkyl, that may be cyclic and may be unsaturated (for example C1-C10 or even C1-C4), or a substituted or unsubstituted aryl or heteroaryl.

Alternatively the substituent groups at position -a may have selenium bonding to the phosphorus. For example, the substituent group may be an R^{x} group bonded to the phosphorus atom via a selenium atom. i.e. —Se—R^{x}. R^{x} can be a substituted or unsubstituted primary, secondary or tertiary alkyl, that may be cyclic and may be unsaturated (for example C1-C10 or even C1-C4), or a substituted or unsubstituted aryl or heteroaryl.

Alternatively the substituent groups at position -a may have nitrogen bonding to the phosphorus. For example, the substituent group may be R^{x} and R^{y} groups bonded to the phosphorus atom via a nitrogen atom. i.e. —N—R^{x}R^{y}. R^{x} and R^{y} can be the same or different and can each independently be a substituted or unsubstituted primary, secondary or tertiary alkyl, that may be cyclic and may be unsaturated (for example C1-C10 or even C1-C4), a substituted or unsubstituted aryl or heteroaryl.

Thus the substituent groups at position -a may be bonded to the phosphorus atom via a heteroatom, e.g. O, S, Se or N. For example, the substituent group may be R^{x} or R^{x} and R^{y} groups bonded to the phosphorus atom via a heteroatom M, i.e. -M-R^{x}R^{y}ₙ. M can be O, S, Se or N. n is 0 or 1 depending on the valency of M. E.g. when M is O, S or Se, n=0 and when M is N, n=1. R^{x} and R^{y} can each independently be a substituted or unsubstituted primary, secondary or tertiary alkyl, that may be cyclic and may be unsaturated (for example C1-C10 or even C1-C4), a substituted or unsubstituted aryl or heteroaryl.

Alternatively, the substituent group at position -a is oxygen, i.e. an oxygen bonded via a double bond to the phosphorus atom.

Thus the TADF compounds of the invention may be selected from the group consisting of compounds according to formula IIIa and formula IIIb

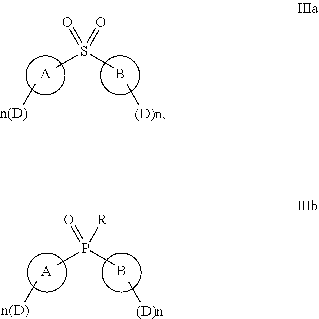

wherein:
rings A and B are aryl and at least one ring is a heteroaryl group Het;
n(D) denotes, independently for each occurrence, n donor groups D bonding to
the respective rings A and B;
n is at least 1 for the at least one group Het; and

R is selected from the group consisting of —H, substituted or unsubstituted primary, secondary or tertiary alkyl, that may be cyclic and may be unsaturated (for example C1-C10 or even C1-C4); substituted or unsubstituted aryl or heteroaryl; substituted or unsubstituted primary, secondary or tertiary alkoxy, that may be cyclic and may be unsaturated (for example C1-C10 or even C1-C4); substituted or unsubstituted aryloxy or heteroaryloxy; substituted or unsubstituted aryl hydroxyl; and substituted or unsubstituted thioalkyl or thioaryl.

The substituent groups R may have oxygen bonding to the phosphorus. For example, substituents at position R may be substituted or unsubstituted primary, secondary or tertiary alkoxy, that may be cyclic and may be unsaturated (for example C1-C10 or even C1-C4); substituted or unsubstituted aryloxy or heteroaryloxy and substituted or unsubstituted aryl hydroxyl. Sometimes, R is a substituted or unsubstituted primary, secondary or tertiary alkoxy, that may be cyclic and may be unsaturated (for example C1-C10 or even C1-C4). Often, the substituted or unsubstituted primary, secondary or tertiary alkoxy is linear. Typically, the primary, secondary or tertiary alkoxy is unsubstituted and is often ethyloxy. Often, R has oxygen bonding to the phosphorus, e.g. as described above, and the phosphorus containing compound of the invention is a phosphinate.

Alternatively, R may have sulfur bonding to the phosphorus. The substituent group may be a substituted or unsubstituted thioalkyl or thioaryl. For example, the substituent group may be an R^{x} group bonded to the phosphorus atom via a sulfur atom. i.e. —S—R^{x}. R^{x} can be a substituted or unsubstituted primary, secondary or tertiary alkyl, that may be cyclic and may be unsaturated (for example C1-C10 or even C1-C4), or a substituted or unsubstituted aryl or heteroaryl.

Alternatively, R may have selenium bonding to the phosphorus. For example, the substituent group may be an R^{x} group bonded to the phosphorus atom via a selenium atom. i.e. —Se—R^{x}. R^{x} can be a substituted or unsubstituted primary, secondary or tertiary alkyl, that may be cyclic and may be unsaturated (for example C1-C10 or even C1-C4), or a substituted or unsubstituted aryl or heteroaryl.

Alternatively R may have nitrogen bonding to the phosphorus. For example, the substituent group may be R^{x} and R^{y} groups bonded to the phosphorus atom via a nitrogen atom. i.e. —N—R^{x}R^{y}. R^{x} and R^{y} can be the same or different and can each independently be a substituted or unsubstituted primary, secondary or tertiary alkyl, that may be cyclic and may be unsaturated (for example C1-C10 or even C1-C4), a substituted or unsubstituted aryl or heteroaryl.

Thus R may be bonded to the phosphorus atom via a heteroatom, e.g. O, S, Se or N. For example, the substituent group may be R^{x} or R^{x} and R^{y} groups bonded to the phosphorus atom via a heteroatom M, i.e. -M-R^{x}R^{y}ₙ. M can be O, S, Se or N. n is 0 or 1 depending on the valency of M. E.g. when M is O, S or Se, n=0 and when M is N, n=1. R^{x} and R^{y} can each independently be a substituted or unsubstituted primary, secondary or tertiary alkyl, that may be cyclic and may be unsaturated (for example C1-C10 or even C1-C4), a substituted or unsubstituted aryl or heteroaryl.

Alternatively, R is oxygen, i.e. an oxygen bonded via a double bond to the phosphorus atom

Where substituents R in formula IIIb are aryl or heteroaryl, they may be substituted with one or more donor groups D. Thus compounds according to formula IIIc are also contemplated:

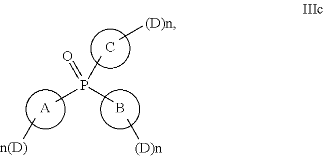

wherein:
rings A, B and C are aryl and at least one ring is a heteroaryl group Het;
n(D) denotes, independently for each occurrence, n donor groups D bonding to the respective rings A, B and C; and
n is at least 1 for the at least one group Het

Where donor groups D are present on a heteroaryl or aryl group of a TADF compound of the invention, the number n may be 1, 2, 3 etc. The maximum number of groups D possible on a heteroaryl or aryl group being determined by the number of bonding positions available on the ring system.

The heteroaryl and aryl groups (where present) may be six membered rings. The heteroaryl and aryl groups (where present) may be six membered rings containing one or more nitrogen atoms, for example 1, 2 or 3 nitrogen atoms; or even 1, 2, 3 or 4 nitrogen atoms. Each Het group can be independently selected from the group consisting of pyridinyl, pyridazinyl, pyrimidinyl, tetrazinyl, pyrazinyl, 1,2,4-triazinyl and 1,3,5-triazinyl. Sometimes, each Het group is independently selected from the group consisting of pyridinyl, pyridazinyl, pyrimidinyl, tetrazinyl, and pyrazinyl. Other times, each Het group is independently selected from the group consisting of pyridazinyl, pyrimidinyl, tetrazinyl, pyrazinyl, 1,2,4-triazinyl and 1,3,5-triazinyl. Often, each Het group is independently selected from the group consisting of 3-pyridinyl, 4-pyridinyl, pyridazinyl, pyrimidinyl, tetrazinyl, and pyrazinyl. Sometimes, each Het group is independently selected from the group consisting of pyridazinyl, pyrimidinyl, tetrazinyl, and pyrazinyl.

Thus the compounds of the invention may be TADF compounds selected from the group consisting of compounds according to formula IVa and formula IVb:

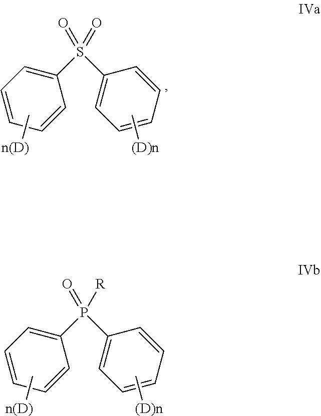

wherein at least one of the positions in one of the six membered rings is a heteroatom;

n(D)- denotes the presence of n donor groups D each bonded to a carbon atom in the respective ring; wherein n is at least 1 for one of the rings; and R has the same meaning as for formula IIIb.

The position of the heteroatom in at least one of the six membered rings in formulas IVa and IVb is not restricted. For example where pyridyl rings are employed in the compounds of the invention the nitrogen atom may be at any position not bonding to the acceptor moiety or a donor group D.

Where no heteroatom is present in a ring n may be from 0 to 5. Where one heteroatom is present in a ring n may be from 0 to 4. However, n is at least 1 for one heteroaryl ring in the structure.

Carbon atoms in the six membered rings of formulas IVa, IVb (or more generally in rings A and B of compounds of formulas IIIa, IIIb) that are not bonded to a donor group D or to the acceptor moieties

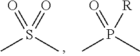

may be H or may be independently substituted. When substituted they may be substituted with substituted or unsubstituted primary, secondary or tertiary alkyl, that may be cyclic and may be unsaturated (for example C1-C10 or even C1-C4); substituted or unsubstituted aryl or heteroaryl, —CF₃, —OMe, —SF₅, —NO₂, halo (e.g. fluoro, chloro, bromo and iodo), aryl, aryl hydroxy, amino, alkoxy, alkylthio, carboxy, cyano, thio, formyl, ester, acyl, thioacyl, amido, sulfonamido, carbamate and the like. Where the substituent is amino it may be NH₂, NHR or NR₂, where the substituents R on the nitrogen may be alkyl, aryl or heteroaryl (for example substituted or unsubstituted C1-C20 or even C1-C10).

Examples of compounds making use of nitrogen heterocycle rings as groups Het are in accordance with formulas IIId, IIIe and IIIf:

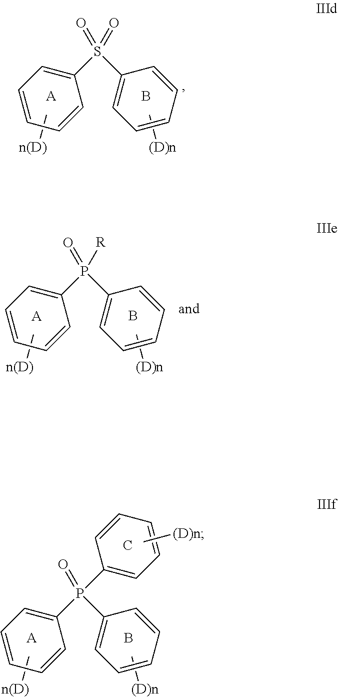

wherein at least one of the positions in one of the six membered rings A, B and C is a nitrogen atom;
n(D)- denotes the presence of n donor groups D each bonded to a carbon atom in the respective ring; and
wherein n is at least 1 for one of the rings that contains a nitrogen atom; and
R has the same meaning as for formula IIIb.

Thus rings A, B and C may (independently) contain from 0 to 3 nitrogen atoms, provided at least one of the rings does contain a nitrogen atom and is substituted with a donor group D.

Examples of compounds of formula Va making use of nitrogen heterocycle rings as groups Het are:

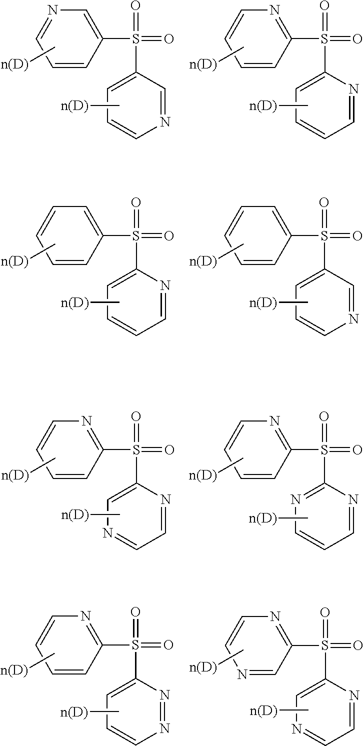

n=0, 1, 2, 3, 4 or 5 provided n=1 for at least one ring containing a nitrogen

Examples of compounds of formula IVb making use of nitrogen heterocycle rings as groups Het are:

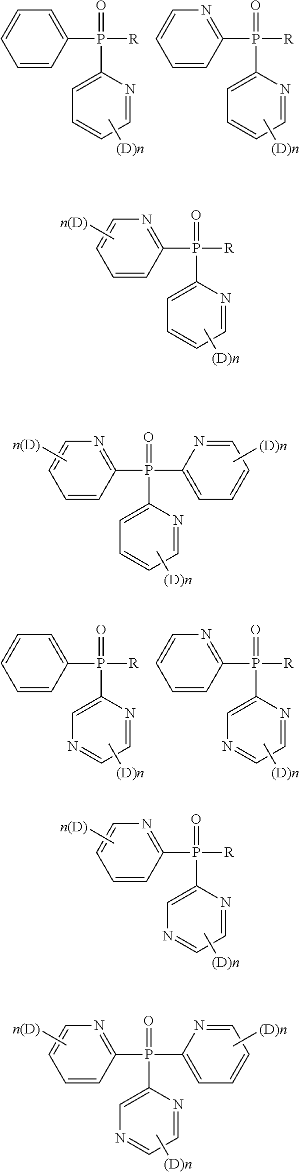

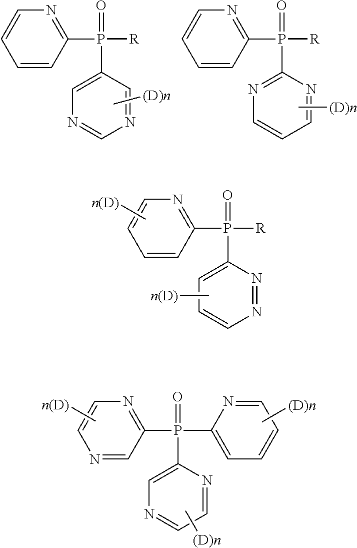

n=0, 1, 2, 3, 4 or 5 provided n=1 for at least one ring containing a nitrogen
R has the same meaning as for formula IlIb.

In the above examples benzene rings are employed where an aryl (rather than heteroaryl) ring is used at one of the bonding positions to the acceptor moieties

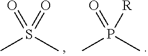

Typically one donor group D may be employed to bond to each of the six membered rings in compounds of formula Iva or IVb. Bonding in the position para to the acceptor moiety can have advantages as discussed hereafter and with reference to specific embodiments. However, other positions may be employed and may aid in adjusting the photo luminescent properties of the compounds concerned.

Thus compounds of formula Ia, Ib may be selected from the group consisting of compounds according to formula Va, Vb, VIa and VIb:

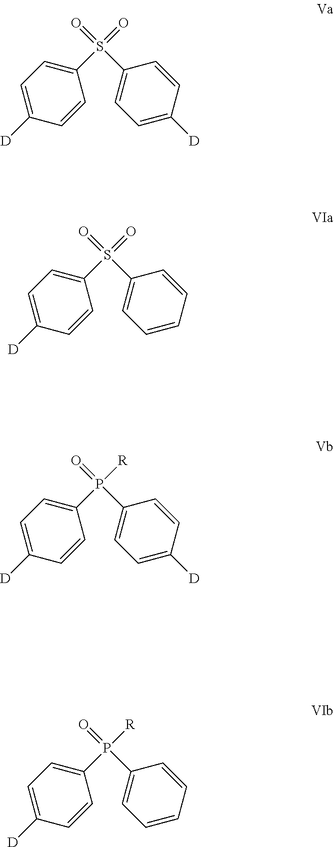

wherein D are donor groups;

wherein at least one of the positions in a six membered ring including a group D is a heteroatom; and R has the same meaning as for compounds of formula IIIb.

As with compounds according to formula Iva, IVb, for compounds according to formula Va, Vb, Via and VIb carbon atoms in the six membered rings not bonded to a donor group D or to the acceptor moieties

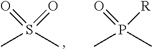

may be H or may be independently substituted. The substituents may be the same as described for compounds of formula I.

Thus sulphone compounds of formulas VII to XV, where D are donor groups, are contemplated.

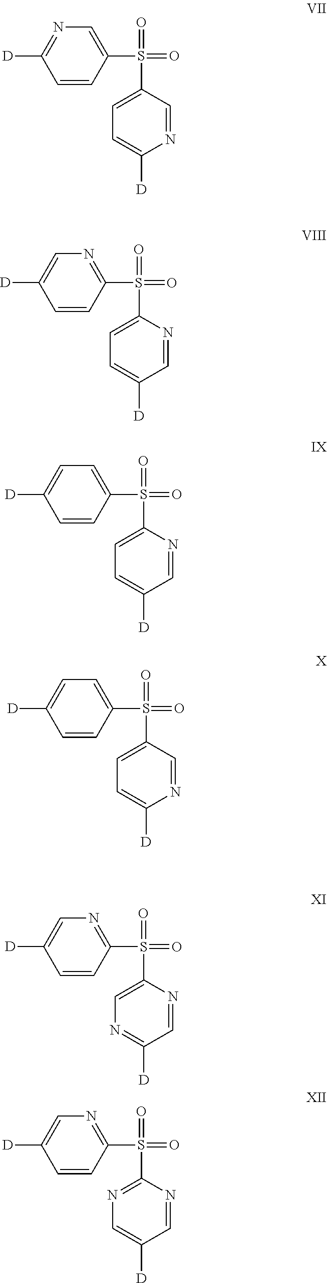

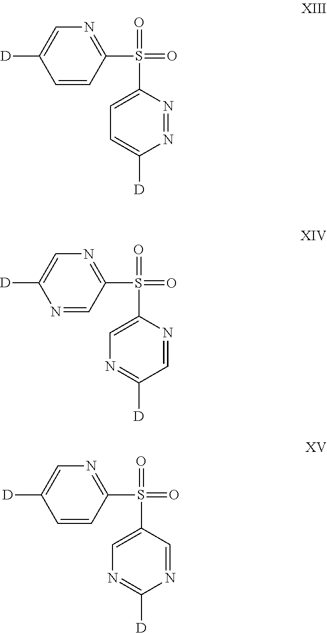

Also contemplated are phosphorus compounds of formulas XVI to XVII

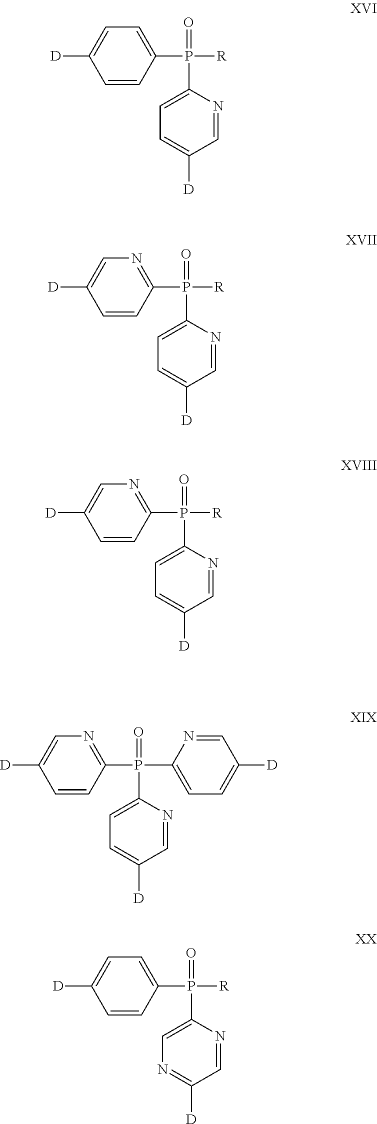

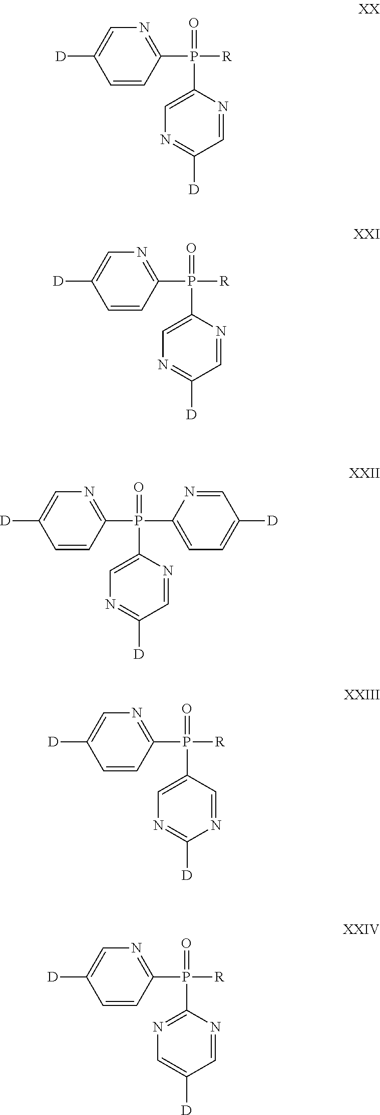

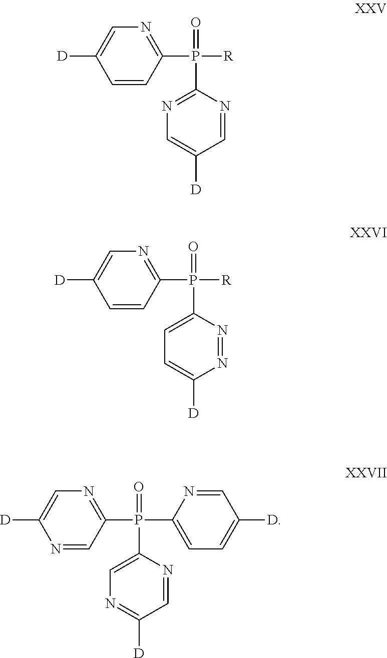

wherein R has the same meaning as for compounds of formula IIIb

Other heteroaryl groups Het and aryl groups may be employed in compounds of the invention. They may be substituted or unsubstituted.

Heteroatoms may be independently selected from N, O and S. More than one heteroatom may be employed in a heteroaryl group Het.

Further examples of heteroaryl group Het include pyridazinyl (in which 2 nitrogen atoms are adjacent in an aromatic 6-membered ring); pyrazinyl (in which 2 nitrogens are 1,4-disposed in a 6-membered aromatic ring); pyrimidinyl (in which 2 nitrogen atoms are 1,3-disposed in a 6-membered aromatic ring); or 1,3,5-triazinyl (in which 3 nitrogen atoms are 1,3,5-disposed in a 6-membered aromatic ring). 5-membered rings are also contemplated, such as pyrazole, 1,2,3 and 1,2,4 triazole, oxazole, oxadiazole, thiazole, thiadiazole and imidazole. Other heterocycles Het may include benzimidazole indole, quinoline, benzothiazole, purine, thiophene, benzothiophene, oxadiazole, benzoxadiazole, thiazole, quinazoline, phthalazine and pteridine.

Sometimes, when the TADF compound of the invention is a sulfone of formula Ia, IIa, IIIa, IVa IIId or Va, Het is not 1,2,4-triazinyl or 1,3,5-triazinyl, i.e. such trazinyl groups are excluded. Sometimes, when the compound of the invention is a sulfone of formula Ia, IIa, IIIa, IVa IIId or Va, Het is not pyridyl. Typically, Het is not 2-pyridyl, i.e. the sulfur atom of the sulfone is not positioned ortho to the nitrogen atom of the pyridyl.

Sometimes, the TADF compound of the invention is either a sulfone of formula Ia, IIa, IIIa, IVa IIId or Va, in which the sulfur atom is bonded to two Het groups, or a phosphinate of formula Ib, IIb, IIIb, IVb, IIIe, Vb, or VIb, in which the substituent groups at position -a or R may have oxygen bonding to the phosphorus, which is bonded to at least one Het group. When there are two Het groups present, each Het can be the same or different.

A wide range of donor groups D for TADF compounds are known.

Carbazole based donor groups may be employed, for example D may be a donor group of the form;

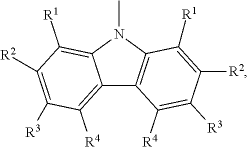

wherein each group R¹, R², R³ and R⁴ is, independently for each occurrence, selected from the group consisting of —H, substituted or unsubstituted primary, secondary or tertiary alkyl, that may be cyclic and may be unsaturated (for example C1-C10 or even C1-C4); substituted or unsubstituted aryl or heteroaryl, —CF₃, —OMe, —SF₅, —NO₂, halo (e.g. fluoro, chloro, bromo and iodo), aryl, aryl hydroxy, amino, alkoxy, alkylthio, carboxy, cyano, thio, formyl, ester, acyl, thioacyl, amido, sulfonamido, carbamate, phosphine oxide, phosphine sulphide and the like.

Where a group R¹, R², R³ or R⁴ is amino it may be —NH₂, —NHR or —NR₂, where the substituents R on the nitrogen may be alkyl, aryl or heteroaryl (for example substituted or unsubstituted C1-C20 or even C1-C10).

Where a group R¹, R², R³ or R⁴ is phosphine oxide or phosphine sulphide it may be selected from the group consisting of:

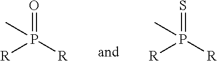

where the substituents R on the phosphorus may be substituted or unsubstituted alkyl, aryl or heteroaryl (for example substituted or unsubstituted C1-C20 or even C1-C10).

The phosphine oxide or phosphine sulphide substituent may be para to the nitrogen of the carbazole structure i.e. one or both of R³ may be a phosphine oxide or phosphine sulphide substituent. Conveniently where both R³ are a phosphine oxide or phosphine sulphide substituent they may be the same. The phosphine oxide or phosphine sulphide substituent may have phenyl or substituted phenyl groups R on the phosphorus.

Thus substituents:

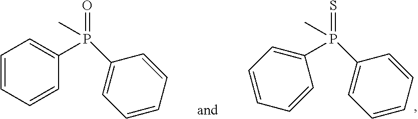

or substituents where one or both phenyl groups are substituted, are contemplated for donor groups D.

More generally donor groups D may also be selected from the following:

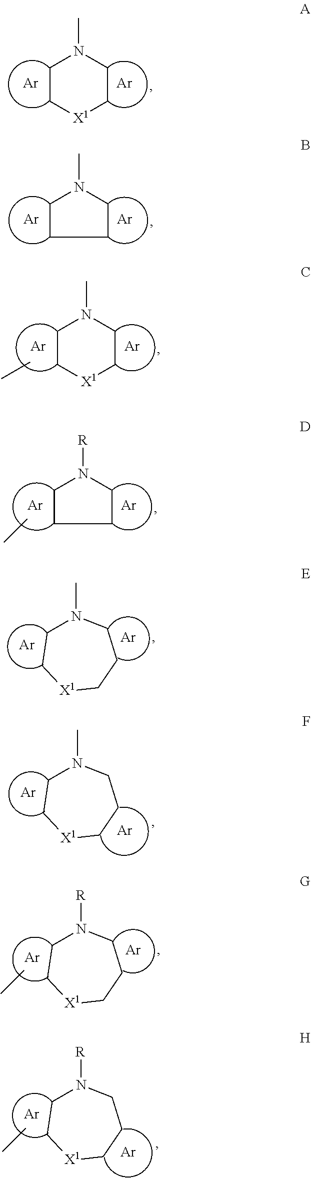

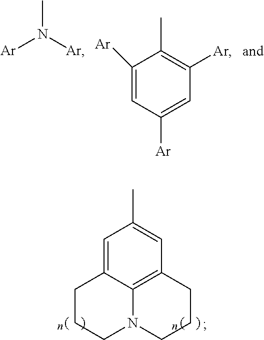

wherein X¹ is selected from the group consisting of O, S, NR, SiR₂, PR and CR₂, wherein each R is independently selected from the group consisting of —H, alkyl, aryl or heteroaryl (for example substituted or unsubstituted C1-C20 or even C1-C10);

each Ar is independently for each occurrence selected from the group consisting of substituted or unsubstituted aryl or heteroaryl; and

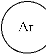

represents, independently for each occurrence a substituted or unsubstituted aryl or heteroaryl ring fused to the central ring of structures A B, C, D, E or F, for example a five or a six membered substituted or unsubstituted aryl or heteroaryl ring, and in structures C, D, G and H bonding to the rest of the molecule is para to the nitrogen;

n ( ) indicates the optional presence of saturated —CH₂— groups in the rings annelated to the benzene ring, wherein n is independently for each occurrence, 0, 1, or 2; substituents on —Ar and

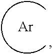

where present can include phosphine oxide or phosphine sulphide, to moderate the donor properties.

Donor groups D in a compound of the invention may also be selected from:

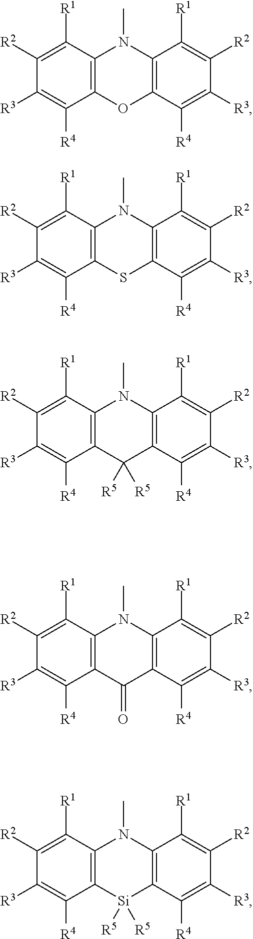

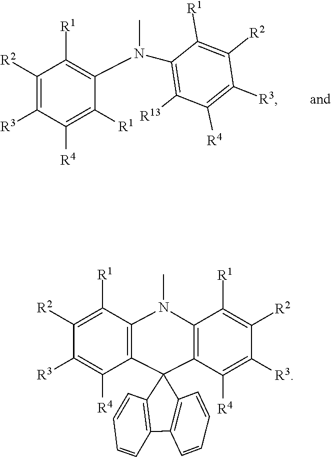

wherein the groups R¹, R², R³ and R⁴ may take the same meaning as before in respect of carbazole based donor groups D; and each group R⁵ may also be independently selected from the same options. Each R⁵ may be alkyl, for example methyl.

Donor groups D in a compound of the invention may also be selected from:

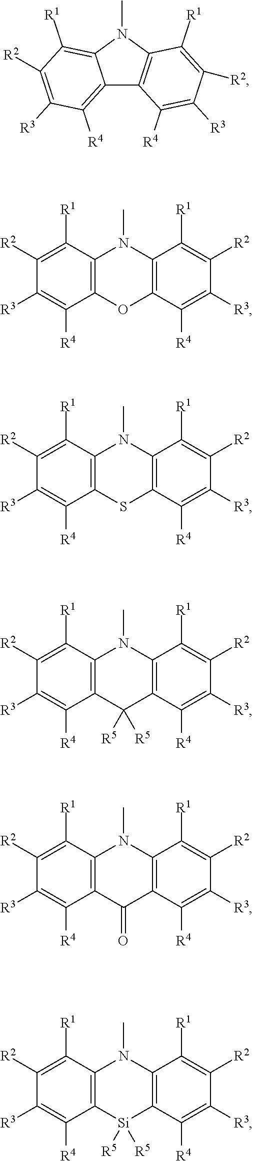

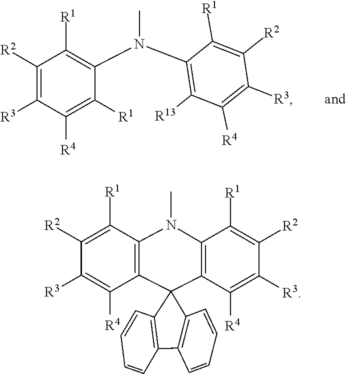

wherein the groups R¹, R², R³ and R⁴ may take the same meaning as before in respect of carbazole based donor groups D; and each group R⁵ may also be independently selected from the same options. Each R⁵ may be alkyl, for example methyl.

In the structure

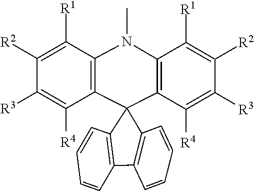

the fluorene moiety may have one or more of the hydrogens substituted by the options indicated for the groups R¹, R², R³ and R⁴.

Examples of the groups R¹, R², R³, R⁴ and R⁵ include the group of alkyl and amino substituents consisting of:

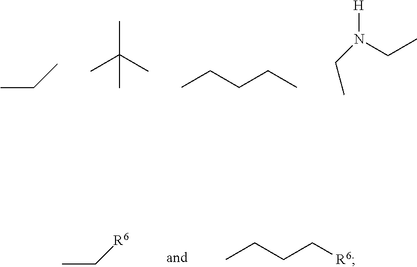

wherein R⁶ may be independently for each occurrence, selected from the group consisting of —H, substituted or unsubstituted primary, secondary or tertiary alkyl, that may be cyclic and may be unsaturated (for example C1-C10 or even C1-C4); substituted or unsubstituted aryl or heteroaryl, —CF₃, —OMe, —SF₅, —NO₂, halo (e.g. fluoro, chloro, bromo and iodo), aryl, aryl hydroxy, amino, alkoxy, alkylthio, carboxy, cyano, thio, formyl, ester, acyl, thioacyl, amido, sulfonamido, carbamate, phosphine oxide, phosphine sulphide and the like. i.e. R⁶ may be as indicated for groups R¹, R², R³, R⁴ and R⁵.

The saturated rings annelated to the benzene ring in the structure:

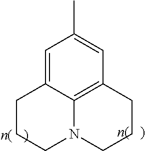

may be five six or seven membered rings. Typically they may be six membered, i.e. the juliolidine structure:

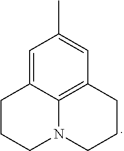

Often, each donor group D of the compound of the invention is not:

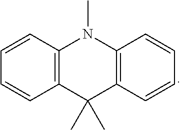 [SMILES: CN1c2ccccc2C(C)(C)c2ccccc21]

Sometimes, donor groups D in a compound of the invention are selected from:

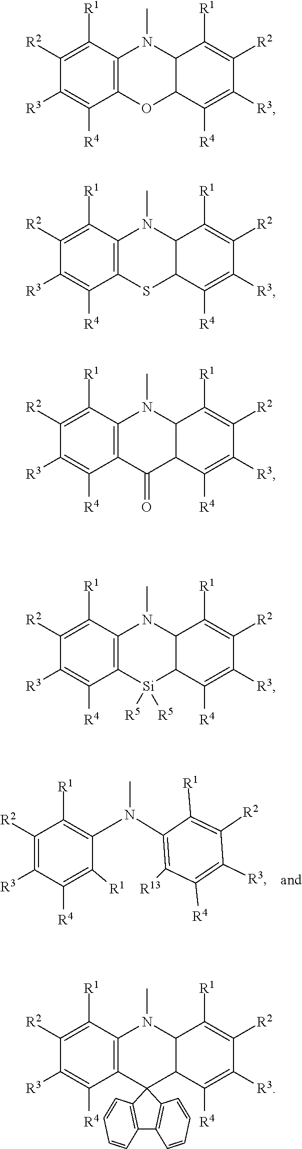

wherein the groups R¹, R², R³ and R⁴ may take the same meaning as before in respect of carbazole based donor groups D; and each group R⁵ may also be independently selected from the same options. Each R⁵ may be alkyl, for example methyl.

Other times, donor groups D in a compound of the invention are selected from:

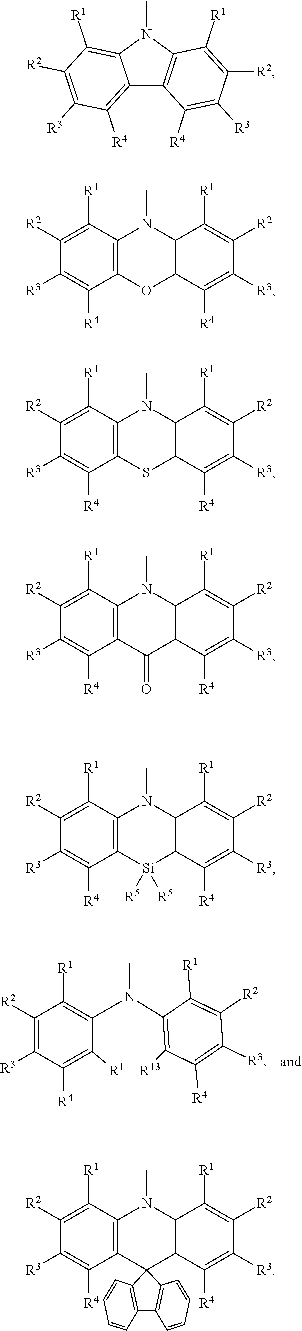

wherein the groups R¹, R², R³ and R⁴ may take the same meaning as before in respect of carbazole based donor groups D; and each group R⁵ may also be independently selected from the same options. Each R⁵ may be alkyl, for example methyl.

Where carbazole based donor groups D are employed they may be substituted at one or both positions para to the nitrogen (R³) and the other positions may be H. The para, (R³) position or positions may be substituted with an alkyl group. for example substituted or unsubstituted primary, secondary or tertiary alkyl, that may be cyclic and may be unsaturated (for example C1-C10 or even C1-C4).

Thus donor groups D may be, for example t-butyl para (to the nitrogen) substituted carbazole or even carbazole para (to the nitrogen) substituted carbazole i.e.:

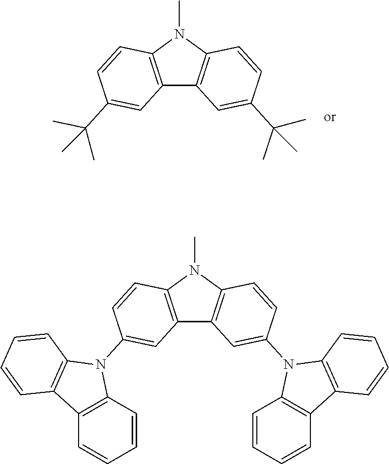 [SMILES: Cn1c2ccc(C(C)(C)C)cc2c2cc(C(C)(C)C)ccc21 ; Cn1c2ccc(-n3c4ccccc4c4ccccc43)cc2c2cc(-n3c4ccccc4c4ccccc43)ccc21]

with bonding in compounds of the invention via the carbazole nitrogen to the acceptor group of formula IIa or IIb.

The sulphone containing compounds of the invention typically have two heteroaryl groups Het bonded to the accepting moiety, one to each of the available bonding positions to S. In addition, or alternatively, the sulphone containing compounds of the invention may not include the compound:

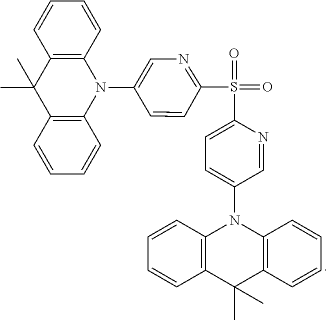 [SMILES: CC1(C)c2ccccc2N(c2ccc(S(=O)(=O)c3ccc(N4c5ccccc5C(C)(C)c5ccccc54)cn3)nc2)c2ccccc21]

In addition, or alternatively, the sulphone containing compounds of the invention may not include compounds which contain a donor group D of formula:

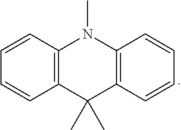 [SMILES: CN1c2ccccc2C(C)(C)c2ccccc21]

In addition, or alternatively, the sulphone containing compounds of the invention may not include compounds which contain two heteroaryl group Hets bonded to the accepting moiety wherein each Het group is 2-pyridyl, i.e. may not include compounds of formula Ia in which the group bonded at position b is Het and each Het is 2-pyridyl. In addition, or alternatively, the sulphone containing compounds of the invention may not include compounds which contain two heteroaryl group Hets bonded to the accepting moiety wherein each Het group is pyridyl, i.e. may not include compounds of formula Ia in which the group bonded at position b is Het and each Het is pyridyl. Sulphone containing TADF compounds of the invention may have a structure selected from the group consisting of:

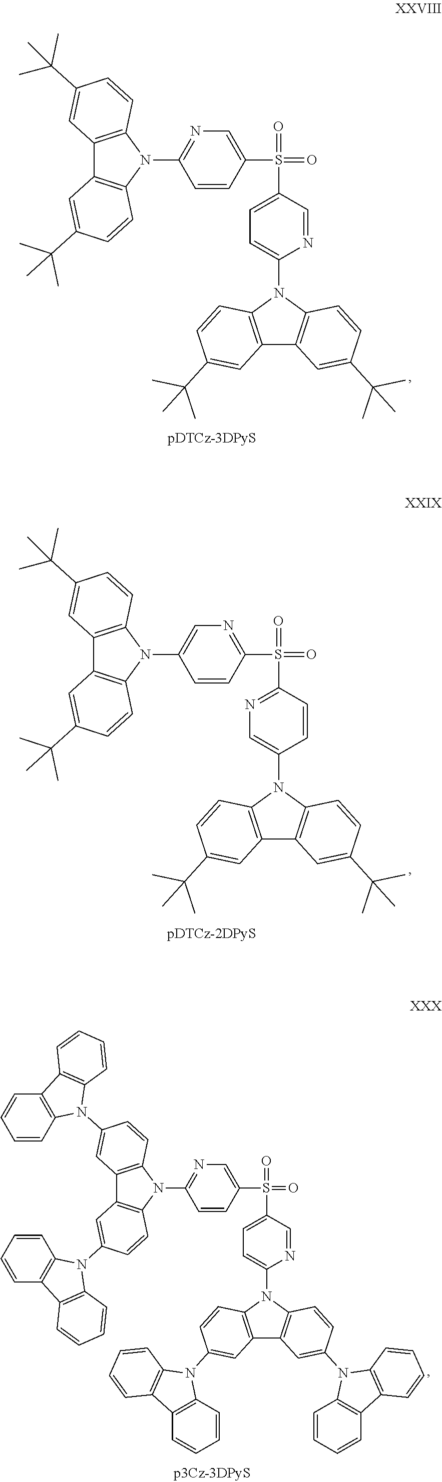

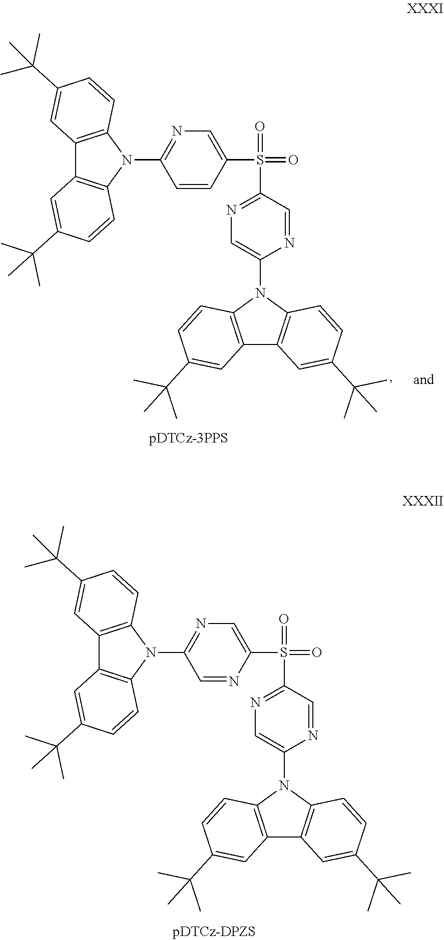

Sulphone containing TADF compounds of the invention may have a structure selected from the group consisting of:

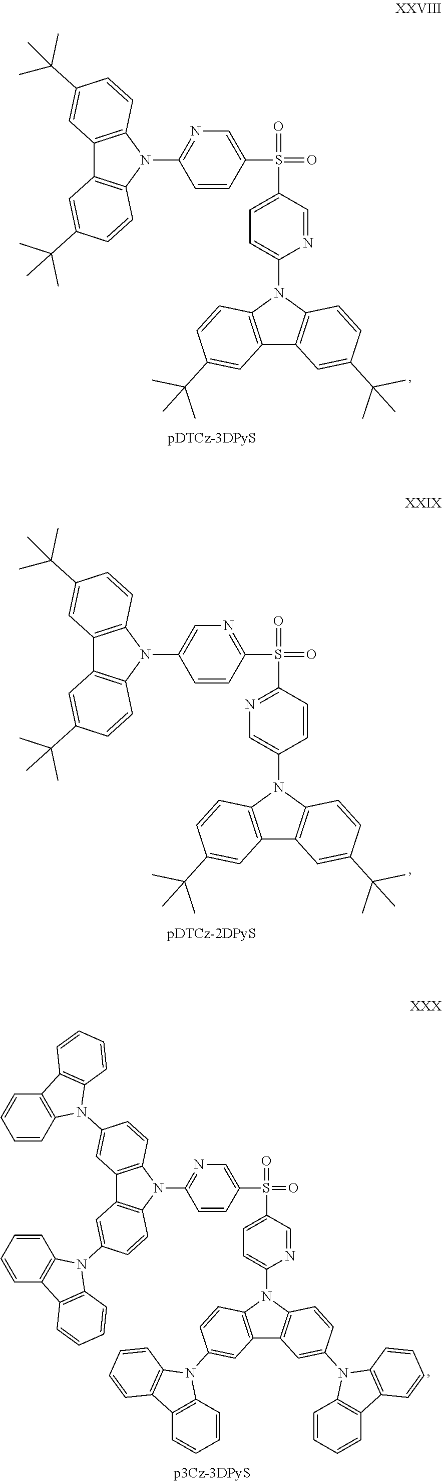

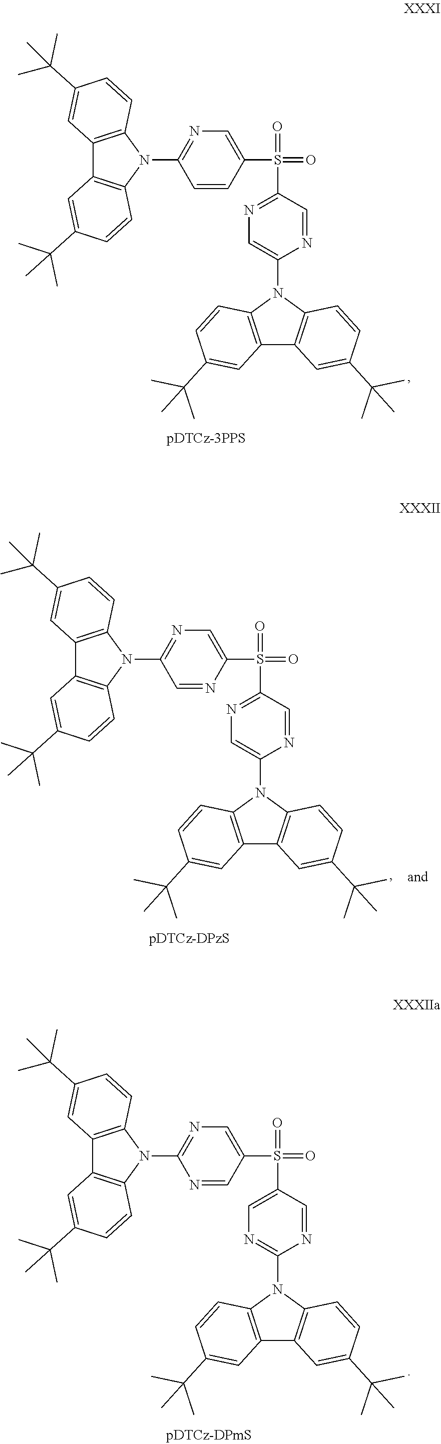

In these examples where the donor groups D are para to the sulphone groups notably good results may be obtained as discussed further hereafter.

Further useful adjustment of the properties of the compounds may be found where a heteroatom, such as N, on the groups Het can interact (for example) by hydrogen bonding with the donor group D. For example the N atoms in the acceptor group heterocycles Het in structure XXVIII are ortho to the carbazole donors D and so can provide this effect.

The phosphorus containing compounds of the invention may not include the compound:

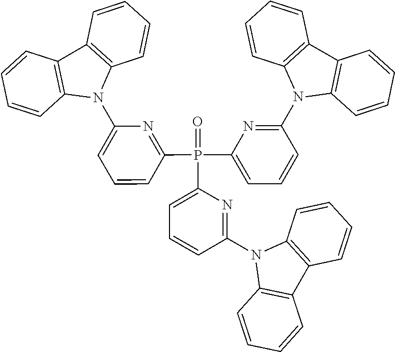 [SMILES: O=P(c1cccc(-n2c3ccccc3c3ccccc32)n1)(c1cccc(-n2c3ccccc3c3ccccc32)n1)c1cccc(-n2c3ccccc3c3ccccc32)n1]

and/or the compound

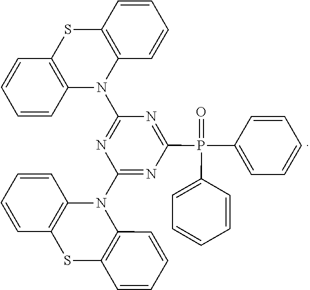 [SMILES: O=P(c1ccccc1)(c1ccccc1)c1nc(N2c3ccccc3Sc3ccccc32)nc(N2c3ccccc3Sc3ccccc32)n1]

In addition or alternatively, the phosphorus containing compounds of the invention may not include compounds which contain a donor group D of formula:

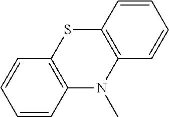

and/or compounds which contain a donor group D containing a substituted or unsubstituted group of formula:

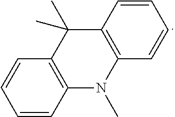 [SMILES: CN1c2ccccc2C(C)(C)c2ccccc21]

Typically, the phosphorus containing compounds of the invention have substituent groups at position -a in formula Ib or R in formula IIIb, IVb, IIIe, Vb and VIb bonded to the phosphorus atom via a heteroatom. The substituent groups may be R^{x} or R^{x} and R^{y} groups bonded to the phosphorus atom via a heteroatom M, i.e. -M-RₓR^{y}ₙ. M can be O, S, Se or N. n is 0 or 1 depending on the valency of M. E.g. when M is O, S or Se, n=0 and when M is N, n=1. R^{x} and R can be each independently a substituted or unsubstituted primary, secondary or tertiary alkyl, that may be cyclic and may be unsaturated (for example C1-C10 or even C1-C4), a substituted or unsubstituted aryl or heteroaryl. The phosphorus containing compounds of the invention can be phosphinates or phosphonates. Typically the phosphorus containing compounds of the invention are phosphinates.

Similarly TADF compounds of the invention containing phosphorus containing acceptor moieties may be selected from the group consisting of:

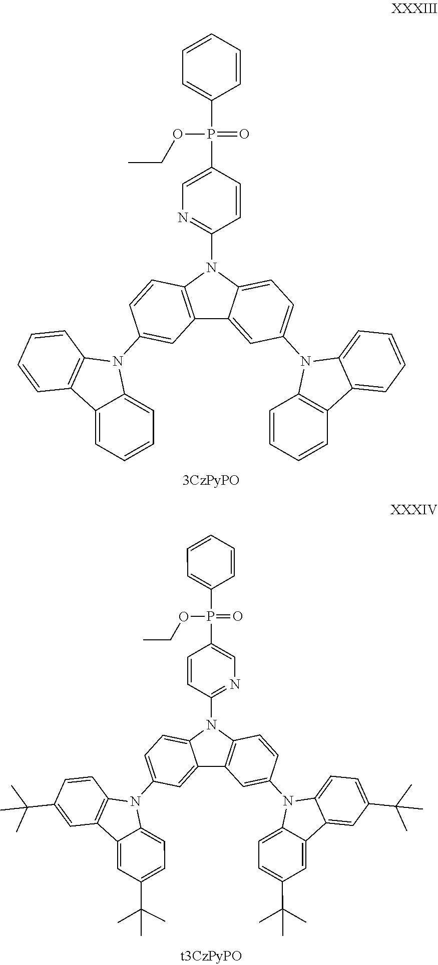 [SMILES: CCOP(=O)(c1ccccc1)c1ccc(-n2c3ccc(-n4c5ccccc5c5ccccc54)cc3c3cc(-n4c5ccccc5c5ccccc54)ccc32)nc1 ; CCOP(=O)(c1ccccc1)c1ccc(-n2c3ccc(-n4c5ccc(C(C)(C)C)cc5c5cc(C(C)(C)C)ccc54)cc3c3cc(-n4c5ccc(C(C)(C)C)cc5c5cc(C(C)(C)C)ccc54)ccc32)nc1]

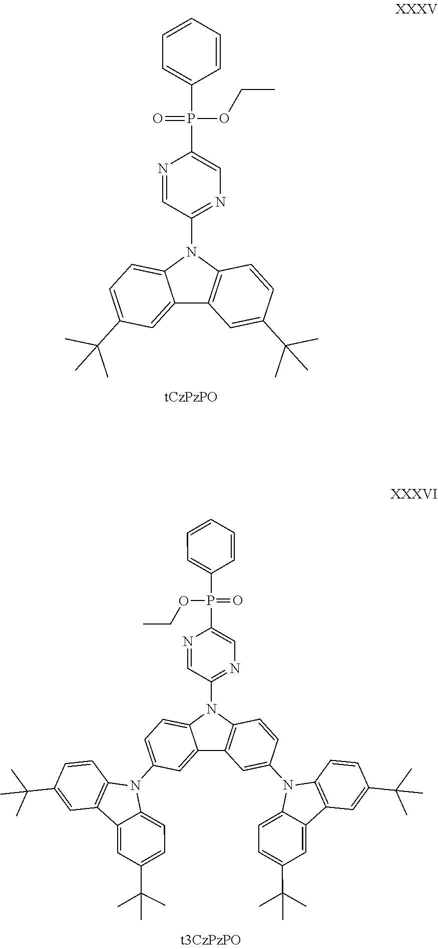 [SMILES: CCOP(=O)(c1ccccc1)c1cnc(-n2c3ccc(C(C)(C)C)cc3c3cc(C(C)(C)C)ccc32)cn1 ; CCOP(=O)(c1ccccc1)c1cnc(-n2c3ccc(-n4c5ccc(C(C)(C)C)cc5c5cc(C(C)(C)C)ccc54)cc3c3cc(-n4c5ccc(C(C)(C)C)cc5c5cc(C(C)(C)C)ccc54)ccc32)cn1]

Compounds of formulas XXXIII to XXXVI make use of phosphinate acceptor moieties. Such compounds can have useful photo physical properties as discussed hereafter with reference to specific examples.

More generally, and without wishing to be bound by theory, the TADF compounds of the invention present relatively rigid structures for TADF materials that can be utilised to manufacture efficient, for example, deep blue-emitting, TADF-based OLEDs. Another design consideration is the orientation of emitter molecules in an emitting film layer. A generally horizontal arrangement can be expected to improve the optical out-coupling efficiency of the OLED. The compounds of the invention can have a generally planar structure which may aid orientation in a film layer of an OLED.

By aryl is meant herein a radical formed formally by abstraction of at least one hydrogen atom from an aromatic compound. As known to those skilled in the art, heteroaryl moieties are a subset of aryl moieties that comprise one or more heteroatoms, typically O, N or S, in place of one or more carbon atoms and any hydrogen atoms attached thereto. Exemplary aryl substituents, for example, include phenyl or naphthyl that may be substituted. Exemplary heteroaryl substituents, for example, include pyridinyl, furanyl, pyrrolyl and pyrimidinyl, which may be substituted

Further examples of heteroaryl rings (which may be substituted) include pyridazinyl (in which 2 nitrogen atoms are adjacent in an aromatic 6-membered ring); pyrazinyl (in which 2 nitrogens are 1,4-disposed in a 6-membered aromatic ring); pyrimidinyl (in which 2 nitrogen atoms are 1,3-disposed in a 6-membered aromatic ring); or 1,3,5-triazinyl (in which 3 nitrogen atoms are 1,3,5-disposed in a 6-membered aromatic ring). Yet further examples of heteroaryl rings include imidazole benzimidazole indole, pyrazole, triazole, oxadiazole, oxazole, thiazole, thiadiazole, quinoline, benzothiazole, purine and pteridine, (all of which may be substituted).

Where groups are substituted herein it is meant (unless otherwise stated or the context dictates otherwise) that groups that may be substituted may be, for example, substituted once, twice, or three times, e.g. once, i.e. formally replacing one or more hydrogen atoms of the group. Examples of such substituents are halo (e.g. fluoro, chloro, bromo and iodo), —SF₅, —CF₃, —OMe, —NO₂, substituted or unsubstituted primary, secondary or tertiary alkyl, that may be cyclic and may be unsaturated (for example C1-C10 or even C1-C4); substituted or unsubstituted aryl or heteroaryl, aryl hydroxy, amino, alkoxy, alkylthio, carboxy, cyano, thio, formyl, ester, acyl, thioacyl, amido, sulfonamido, carbamate, phosphine oxide, phosphine sulphide and the like. Where the substituent is amino it may be NH₂, NHR or NR₂, where the substituents R on the nitrogen may be alkyl, aryl or heteroaryl (for example substituted or unsubstituted C1-C20 or even C1-C10).

Synthesis of organic thermally activated delayed fluorescence (TADF) can be carried out by a skilled person. Examples of synthetic routes are described hereafter and with reference to specific embodiments.

An illustration of the general procedures for compounds employing the sulphone acceptor moiety (e.g compounds of formula IIIa) is provided in Scheme 1 below where carbazole based donor groups D are deployed to make two different TADF compounds. In these examples (described below in more detail under the heading “Detailed Description of Some Embodiments and Experimental Results”) di t-butyl substituted carbazole donor groups are employed to provide XXIX ‘pDTCz-2DPyS’ {9,9′-(sulfonylbis(pyridine-6,3-diyl))bis(3,6-di-tert-butyl-9H-carbazole)} and XXVIII ‘pDTCz-3DPyS’ {9,9′-(sulfonylbis(pyridine-5,2-diyl))bis(3,6-di-tert-butyl-9H-carbazole)}both of which are deep blue emitters. A similar strategy can be used to prepare other related sulphone containing compounds such as XXX to XXXIIa.

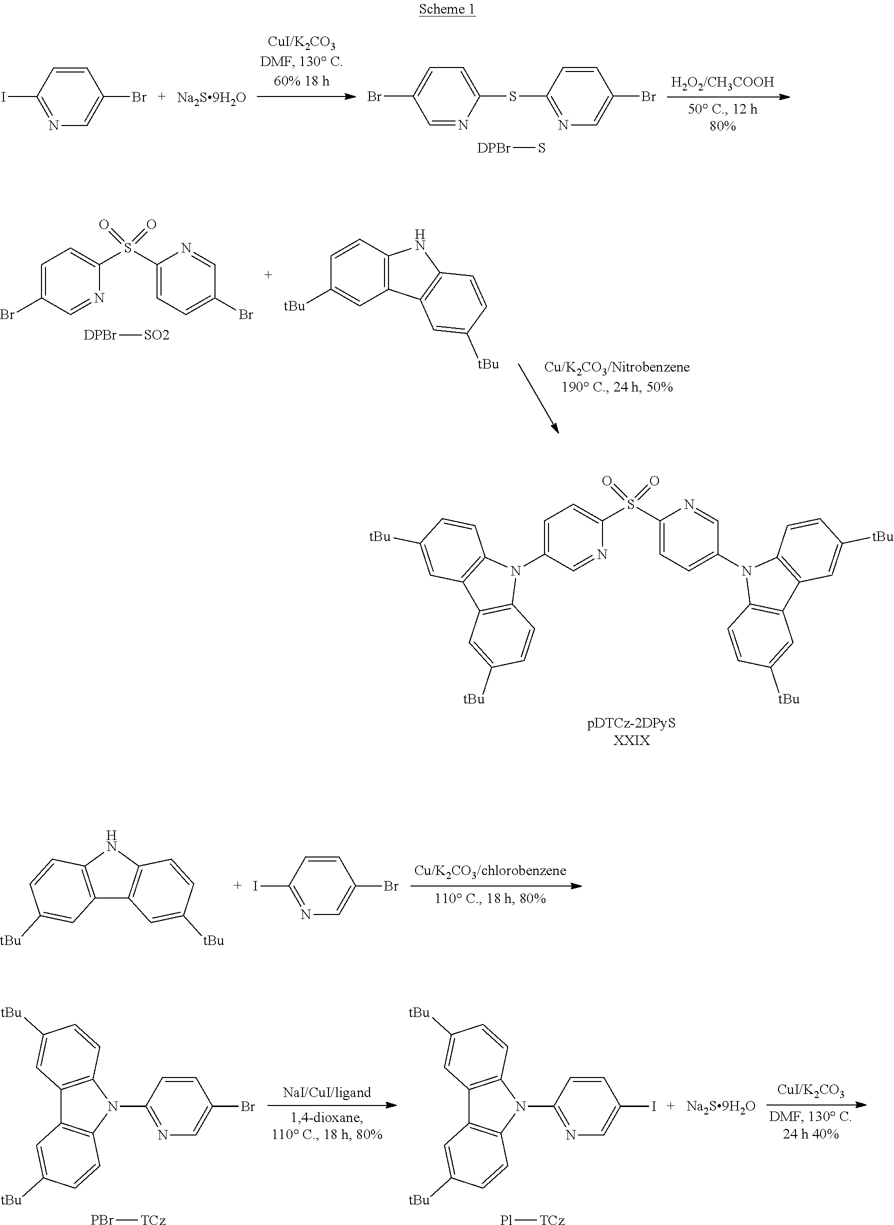

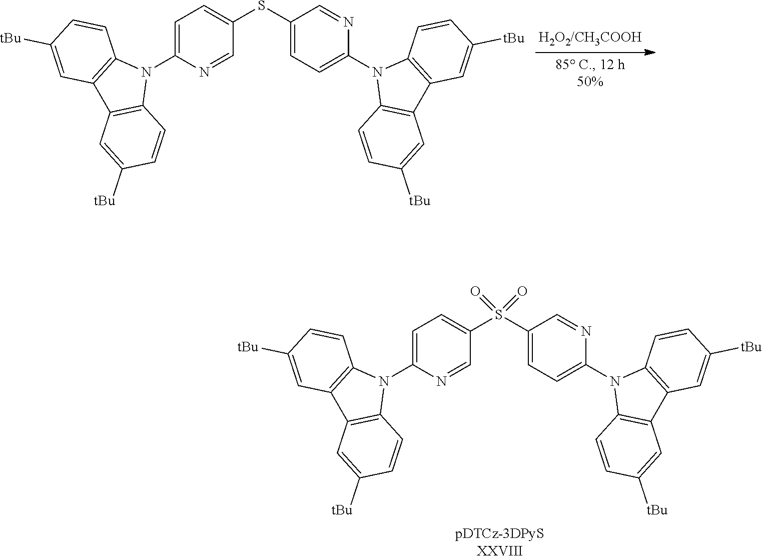 [SMILES: CC(C)(C)c1ccc2c(c1)c1cc(C(C)(C)C)ccc1n2-c1ccc(Sc2ccc(-n3c4ccc(C(C)(C)C)cc4c4cc(C(C)(C)C)ccc43)nc2)cn1 ; CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)nc2)cn1]

The synthesis of some carbazole containing donor groups D is illustrated in Scheme 2 below and described in more detail under the heading Detailed Description of Some Embodiments and Experimental Results, below.

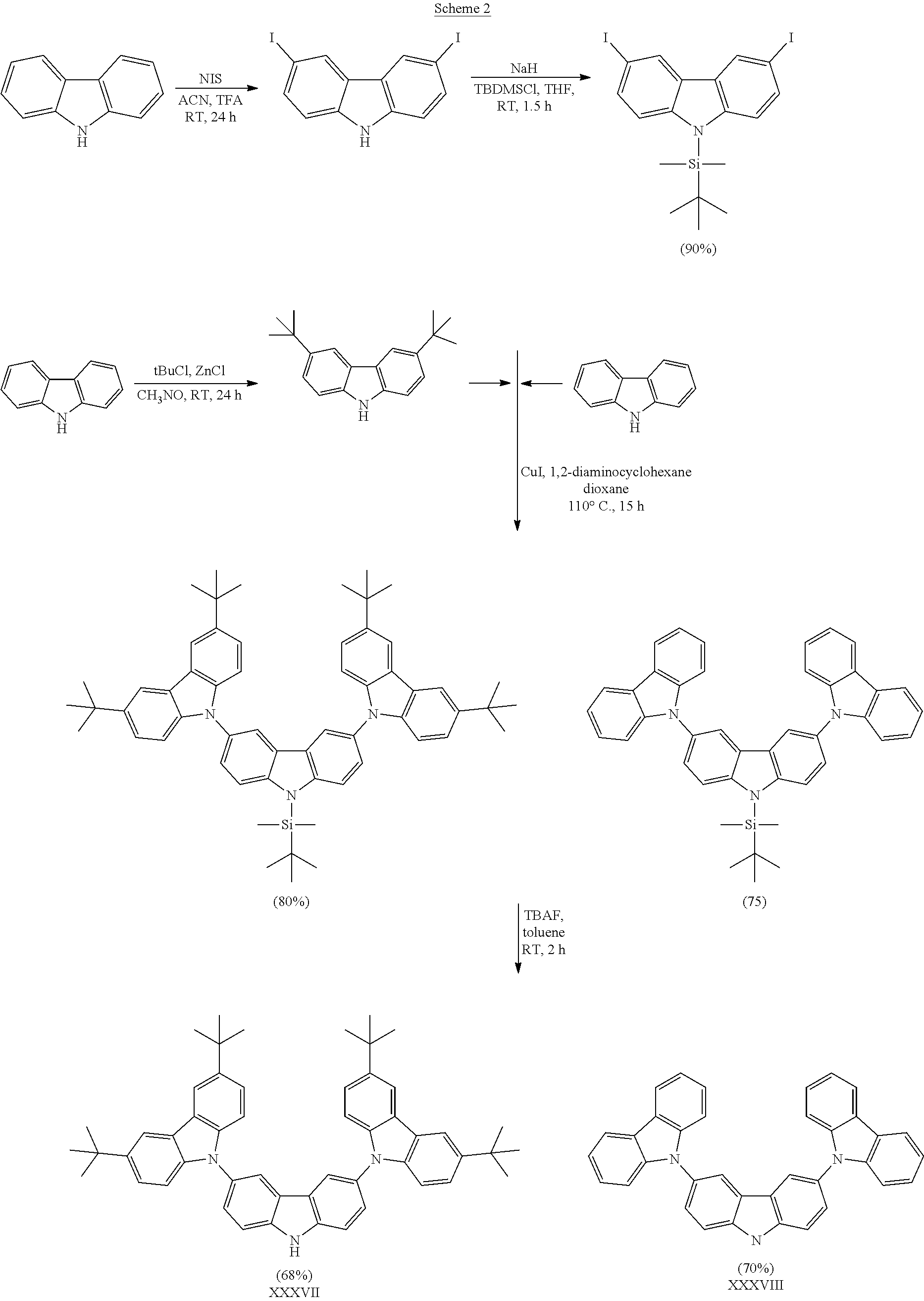

The synthesis of compounds of the invention where the acceptor group comprises phosphorus and making use of the carbazole donors XXXVII and XXXVIII is illustrated in Schemes 3, 4 and 5 below. More detail for the manufacture of XXXIII, XXXIV and XXXVI is given under the heading “Detailed Description of Some Embodiments and Experimental Results”, below.

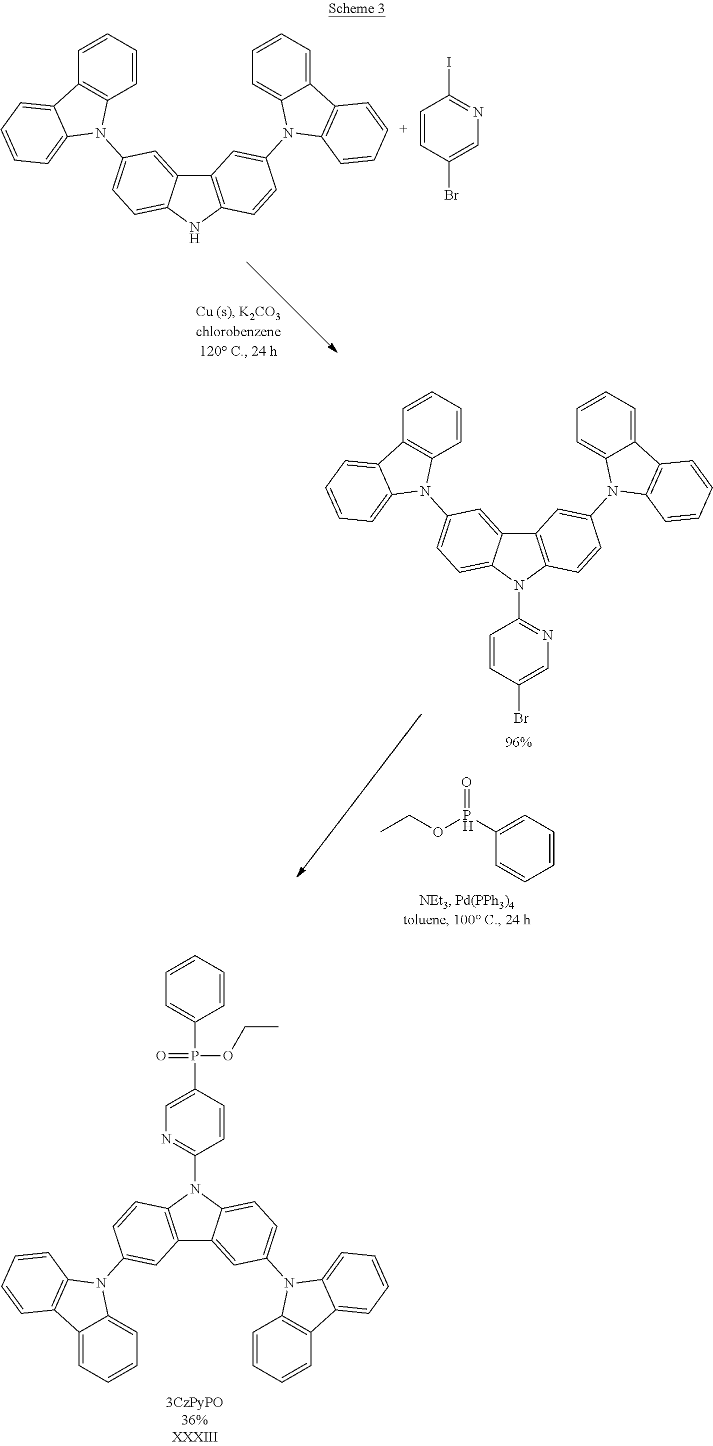

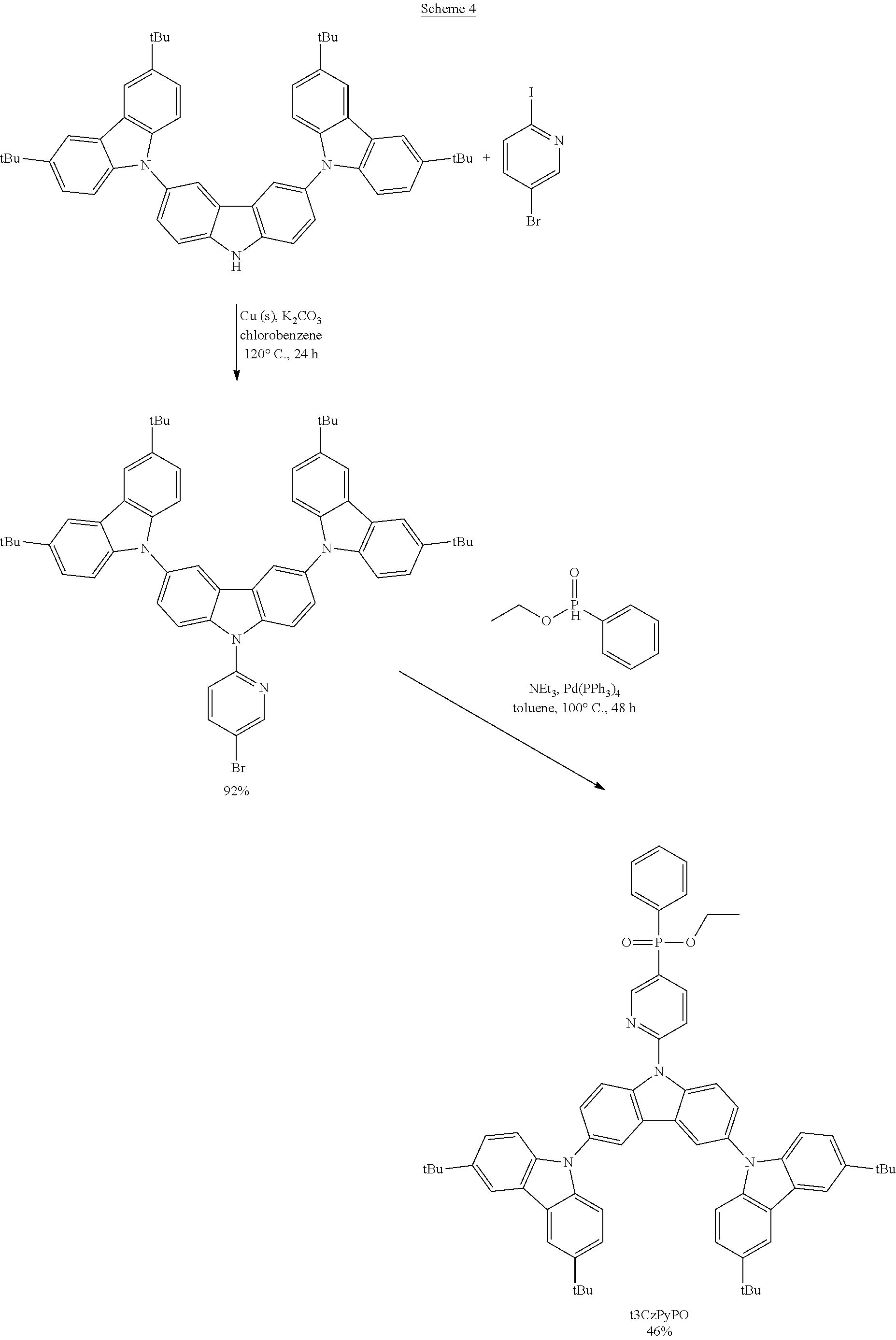

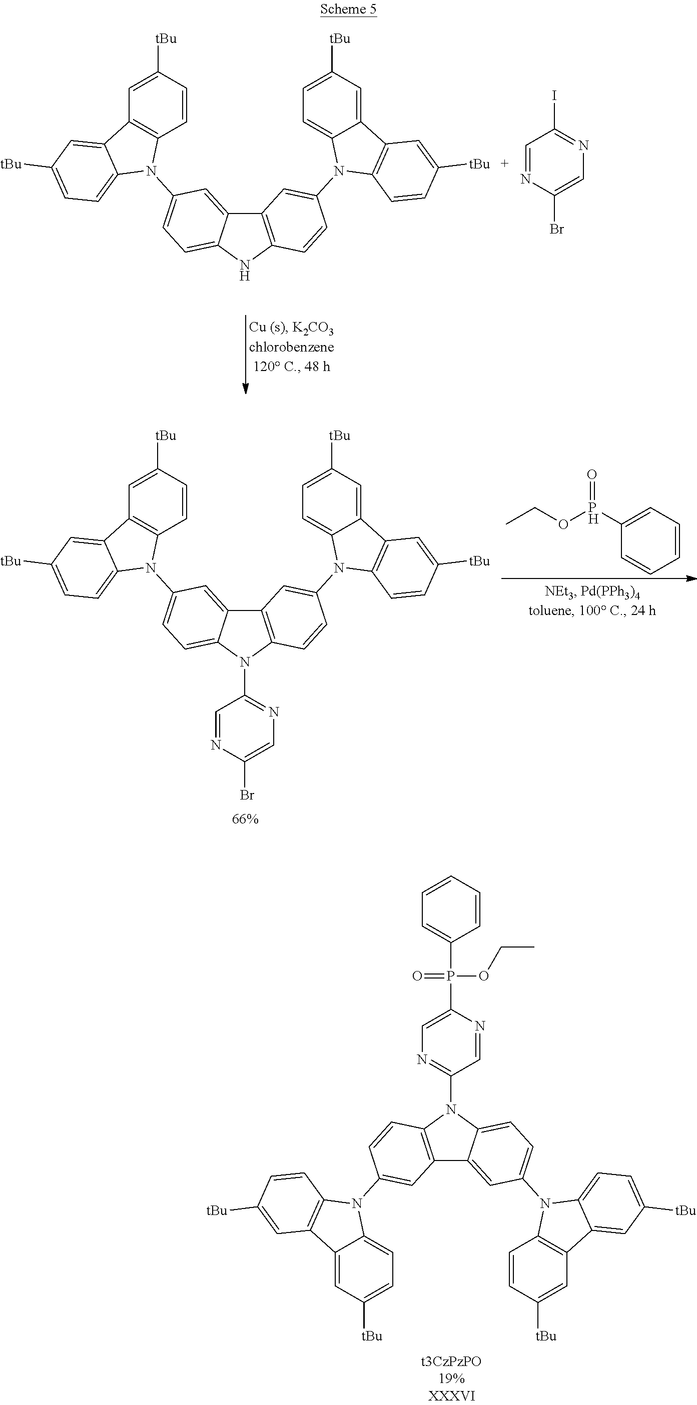

The compounds of the first aspect of the invention can be employed as light emitting materials. Thus according to a second aspect the present invention provides an electroluminescent device such as an OLED or a light emitting electrochemical cell (LEEC) comprising one or more of the compounds according to the first aspect of the invention as an emitter material.

### BRIEF DESCRIPTION OF THE DRAWINGS

FIG. 1 shows absorbance spectra of luminescent compounds DTCz-3DPyS (also referred to as 3DPS-pDTCz or XXVIII) and DTCz-2DPyS (also referred to as 2DPS-pDTCz or XXIX);

FIGS. 2a and 2b show photoluminescence (PL) spectra of compounds pDTCz-2DPyS (also referred to as 2DPS-pDTCz or XXIX) and pDTCz-3DPyS (also referred to as 3DPS-pDTCz or XXVIII), respectively, in various solvents;

FIGS. 3a and 3b show fluorescence (at 300 K) and phosphorescence (at 77 K) emission spectra of pDTCz-2DPyS (also referred to as 2DPS-pDTCz or XXIX) and pDTCz-3DPyS (also referred to as 3DPS-pDTCz or XXVIII), respectively;

FIG. 4 shows cyclic voltammetry traces of pDTCz-2DPyS (also referred to as 2DPS-pDTCz or XXIX) and pDTCz-3DPyS (also referred to as 3DPS-pDTCz or XXVIII);

FIGS. 5a and 5b show transient PL (photo luminescent) decay characteristics of pDTCz-2DPyS (also referred to as 2DPS-pDTCz or XXIX) and pDTCz-3DPyS (also referred to as 3DPS-pDTCz or XXVIII);

FIG. 6a shows absorbance spectra and FIGS. 6b, 6c and 6d show photoluminescence (PL) spectra of pDTCz-2DPyS (also referred to as 2DPS-pDTCz or XXIX) and pDTCz-3DPyS (also referred to as 3DPS-pDTCz or XXVIII) in various solvents;

FIG. 7 shows cyclic voltammetry traces of 3CzPyPO (XXXII), t3CzPyPO (XXXIV) and t3CzPzPO (XXXVI);

FIGS. 8a and 8b show solid state thin film spectra of 3CzPyPO (XXXIII), t3CzPyPO (XXXIV) and t3CzPzPO (XXXVI);

FIG. 9 shows schematically, the layers of an OLED device comprising DTCz-3DPyS (also referred to as 3DPS-pDTCz or XVIII) or DTCz-2DPyS (also referred to as 2DPS-pDTCz or XXIX) as emitter material; and

FIGS. 10a and 10b compare the performance of devices comprising DTCz-3DPyS (also referred to as 3DPS-pDTCz or XVIII), DTCz-2DPyS (also referred to as 2DPS-pDTCz or XXIX) and DPS-pDTCz (reference compound). FIG. 10a contains plots of external quantum efficiency as a function of luminance, and FIG. 10b contains electroluminescence spectra and device photos.

FIGS. 11a to 11c show (a) the electroluminescence (EL) spectrum (b) external quantum efficiencies (EQE) versus brightness and (c) current density (J) and brightness versus voltage (V) curves of devices based on pDTCz-DPmS (also referred to as Pm—SO₂-tCz or XXXIIa). FIGS. 11d to 11f show (d) EL spectrum, (e) EQE versus brightness and (f) J and brightness versus V curves of devices based on pDTCz-DPzS (also referred to as Pz-SO₂-tCz or XXXII).

### DETAILED DESCRIPTION OF SOME EMBODIMENTS AND EXPERIMENTAL RESULTS

Sulphone Containing Compounds

Scheme 1 (above) illustrates synthetic routes. More detailed explanation is provided below, with yields and spectroscopic information.

### bis(5-bromopyridin-2-yl)sulfane (DPBr-S)

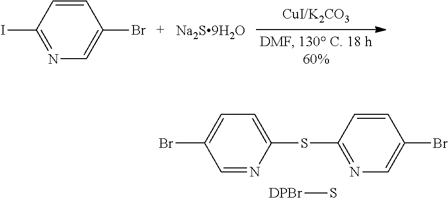

To a 150 mL three neck flask, 2-iodo-5-bromopyridine (280 mg, 1 mmol), sodium sulfide (140 mg, 0.6 mmol), copper (1) iodide (30 mg, 0.1 mmol) and potassium carbonate (140 mg, 1 mmol) were added. The for others I will ask Dongyang flask was degassed by vacuum-nitrogen-reflux for three times and 10 mL of DMF was injected. The mixture was stirred on 130° C. for 18 h under nitrogen. The mixture was washed with water and extracted with ethyl acetate for three times (50 mL×3). The organic solvent was removed by rotary evaporator and the crude product was purified by chromatography. DCM/Hexane=1/1 was used as eluent to obtain DPBr-S as a white solid. Yield 80%, 120 mg. ¹H NMR (400 MHz, CDCl₃) δ 8.61 (s, 2H), 7.77 (dd, J=8.4, 2.4 Hz, 2H), 7.39 (d, J=8.4 Hz, 2H).

### 6,6′-sulfonylbis(3-bromopyridine) (DPBr-SO2)

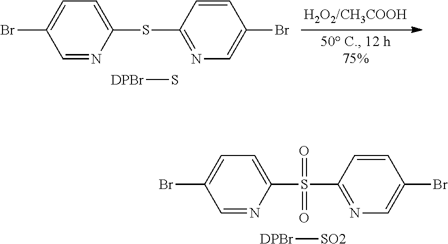

To a 50 mL flask, DPBr-S (60 mg, 0.2 mmol) was dissolved in 2 mL of glacial acetic acid, and 2 mL of hydrogen peroxide solution (30 wt %) was added and the mixture was stirred at 50° C. for 12 h. The mixture was poured into 20 mL of ice cold water and extracted with dichloromethane (DCM) for three times (20 mL×3). The organic solvent was removed by rotary evaporator and the crude product was purified by chromatography. DCM/Hexane=1/1 was used as eluent to obtain DPBr-SO2 as a white solid. Yield 75%, 50 mg. ¹H NMR (400 MHz, CDCl₃) δ 8.72-8.65 (m, 2H), 8.02 (dd, J=8.4, 2.3 Hz, 2H), 7.93 (dd, J=8.4, 0.7 Hz, 2H). HRMS ESI⁺ [M+H]⁺: C₁₀H₇Br₂O₂SN₂ cald for 378.8569, found 378.8567.

### 9,9′-(sulfonylbis(pyridine-6,3-diyl))bis(3,6-di-tert-butyl-9H-carbazole) (pDTCz-2DPyS, also referred to as 2DPS-pDTCz) Compound XXIX

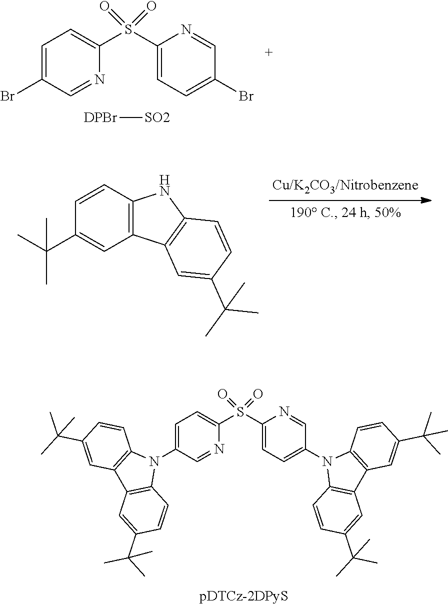

To a 50 mL flask, DPBr-SO2 (50 mg, 0.13 mmol), di-tert-butyl-9H-carbazole (100 mg, 0.3 mmol), copper powder (10 mg, 0.14 mmol) and potassium carbonate (50 mg, 0.4 mmol) were added. The flask was degassed by vacuum-nitrogen and cycles were repeated for three times and 5 mL of nitrobenzene was added. The mixture was stirred at 190° C. for 24 h under nitrogen atmosphere. The mixture was washed by water and extracted by DCM for three time (50 mL×3). The organic solvent was removed by rotary evaporator and the crude product was purified by chromatography. DCM/Hexane=1/2 was used as eluent to get pDTCz-2DPyS as a white solid. Yield 50%, 60 mg. ¹H NMR (400 MHz, CDCl₃) δ 9.08 (dd, J=2.5, 0.7 Hz, 2H), 8.67 (dd, J=8.4, 0.7 Hz, 2H), 8.29 (dd, J=8.4, 2.5 Hz, 2H), 8.16 (dd, J=1.9, 0.7 Hz, 4H), 7.53 (dd, J=8.7, 1.9 Hz, 4H), 7.48 (dd, J=8.6, 0.7 Hz, 4H), 1.49 (s, 36H). HRMS ESI⁺ [M+H]⁺: C₅₀H₅₄N₄O₂SH calced for 775.4040, found 775.4033. HPLC: H₂O (5%)/MeCN, 1.0 mL min⁻¹, 300 nm; tr (99.4%)=1.6 min.

### 9-(5-bromopyridin-2-yl)-3,6-di-tert-butyl-9H-carbazole (PBr-TCz)

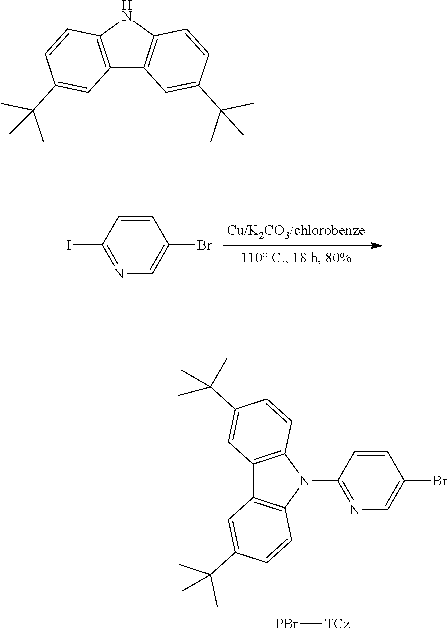

To a 250 mL flask, 2-iodo-5-bromopyridine (1.4 g, 5 mmol), di-tert-butyl-9H-carbazole (1.4 mg, 5 mmol), copper powder (320 mg, 5 mmol) and potassium carbonate (2.2 mg, 15 mmol) were added. The flask was degassed by vacuum-nitrogen and cycles were repeated for three times and 20 mL of chlorobenzene was injected. The mixture was stirred at 110° C. for 18 h under nitrogen atmosphere. The mixture was washed with water and extracted with DCM for three times (50 mL×3). The organic solvent was removed by rotary evaporator and the crude product was purified by chromatography. DCM/Hexane=1/3 was used as eluent to get PBr-TC as a white solid. Yield 80%, 1.2 g. ¹H NMR (400 MHz, CDCl₃) δ 8.75 (dd, J=2.6, 0.7 Hz, 1H), 8.12 (d, J=1.9 Hz, 2H), 8.01 (dd, J=8.6, 2.5 Hz, 1H), 7.80 (dd, J=8.7, 0.6 Hz, 2H), 7.58 (dd, J=8.6, 0.7 Hz, 1H), 7.52 (dd, J=8.7, 2.0 Hz, 2H), 1.49 (s, 18H).

### 9-(5-iodopyridin-2-yl)-3,6-di-tert-butyl-9H-carbazole (PI-TC)

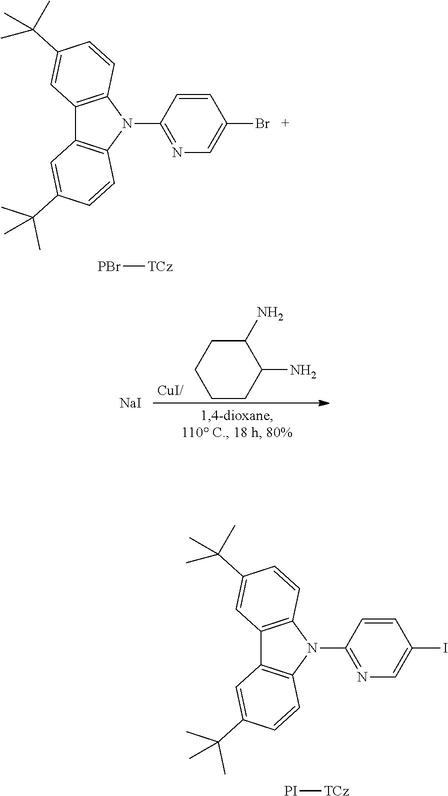

To a 100 mL flask, PBr-TC (250 mg, 0.6 mmol), sodium iodide (150 mg, 1 mmol), and copper (1) iodide (20 mg, 0.1 mmol) were added. The flask was degassed by vacuum-nitrogen for three time and 20 mL of chlorobenzene was injected. The mixture was stirred on 110° C. for 18 h under nitrogen atmosphere. The mixture was washed by water and extracted by DCM for three time (50 mL×3). The organic solvent was removed by rotary evaporator and the crude product was purified by chromatography. DCM/Hexane=1/3 was used as eluent to obtain I-Py-tCz as white solid. Yield 80%, 245 mg. ¹H NMR (400 MHz, CDCl₃) δ 8.89 (dd, J=2.3, 0.7 Hz, 1H), 8.18 (dd, J=8.5, 2.4 Hz, 1H), 8.12 (dd, J=2.0, 0.6 Hz, 2H), 7.81 (dd, J=8.7, 0.6 Hz, 2H), 7.55-7.47 (m, 3H), 1.48 (s, 18H).

### bis(6-(3,6-di-tert-butyl-9H-carbazol-9-yl)pyridin-3-yl)sulfane (pDTCz-3DPyS, also referred to as 3DPS-pDTCz)

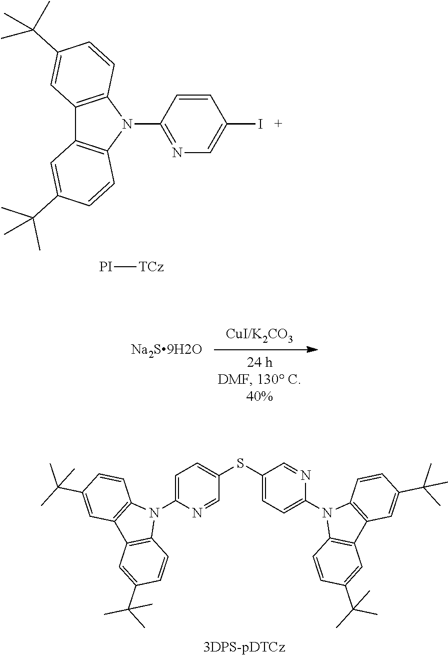

To a 100 mL three necks flask, PI-TC (300 mg, 0.6 mmol), sodium sulfide (70 mg, 0.3 mmol), copper (I) iodide (15 mg, 0.05 mmol) and potassium carbonate (80 mg, 0.7 mmol) were added. The flask was degassed by vacuum-nitrogen for three time and 10 mL of DMF was injected. The mixture was stirred on 130° C. for 24 h under nitrogen atmosphere. The reaction mixture poured to water and extracted with ethyl acetate for three times (20 mL×3). The organic solvent was removed by rotary evaporator and the crude product was purified by column chromatography. DCM/Hexane=1/1 was used as eluent to get 3DPS-pDTC as white solid. Yield 40%, 120 mg. ¹H NMR (400 MHz, CDCl₃) δ 8.78 (dd, J=2.5, 0.7 Hz, 2H), 8.12 (dd, J=2.0, 0.6 Hz, 4H), 7.96 (dd, J=8.5, 2.5 Hz, 2H), 7.87 (dd, J=8.7, 0.6 Hz, 4H), 7.69 (dd, J=8.5, 0.8 Hz, 2H), 7.55-7.49 (m, 4H), 1.49 (s, 36H).

### 9,9′-(sulfonylbis(pyridine-5,2-diyl))bis(3,6-di-tert-butyl-9H-carbazole) (pDTCz-3DPyS, also referred to as 3DPS-pDTCz) Compound XXVIII

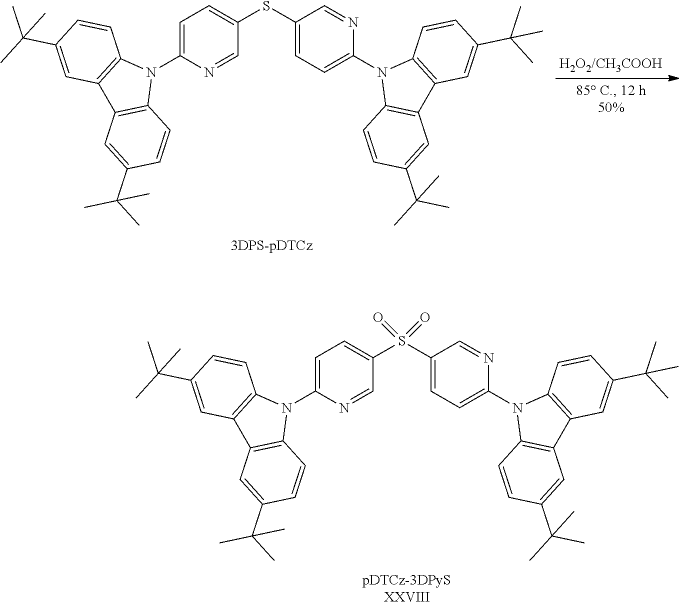

To a 50 mL flask, 3DPS-pDTC (70 mg, 1 mmol) was dissolved in 2 mL of glacial acetic acid, and 2 mL of peroxide hydrogen solution (30 wt %) was added. The reaction mixture was stirred at 50° C. for 12 h. The mixture was poured into 20 mL of ice cold water and extracted with dichloromethane (DCM) for three times (20 mL×3). The organic solvent was removed by rotary evaporator and the crude product was purified by chromatography. DCM/Hexane=1/1 was used as eluent to obtain 3DPS-pDTC as white solid. Yield 50%, 50 mg. ¹H NMR (400 MHz, CDCl₃) δ 9.30 (d, J=2.5 Hz, 2H), 8.43 (dd, J=8.7, 2.6 Hz, 2H), 8.10 (d, J=2.0 Hz, 4H), 7.99 (d, J=8.8 Hz, 4H), 7.87 (d, J=8.7 Hz, 2H), 7.53 (dd, J=8.8, 2.0 Hz, 4H), 1.48 (s, 36H). HRMS ESI⁺ [M+H]⁺: C₅₀H₅₄N₄O₂SH calculated for 775.4040, found 775.4034. HPLC 5% H₂O/MeCN, 1.0 mL min⁻¹, 300 nm; tr (98.2%)=6.1 min.

Photophysical Properties of Sulphone Compounds

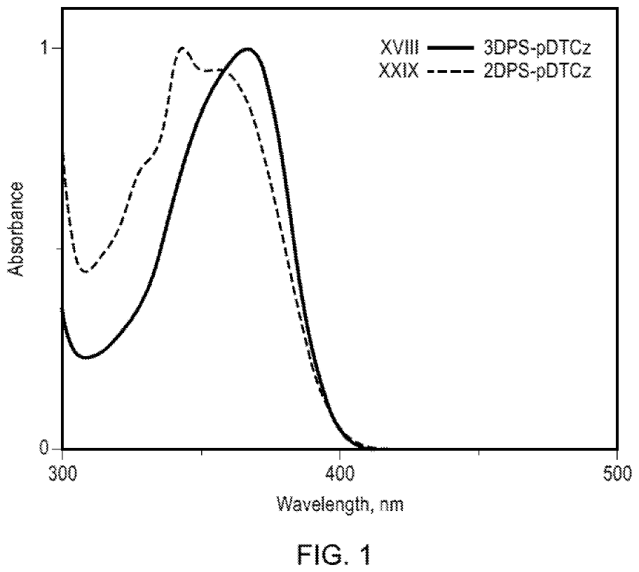

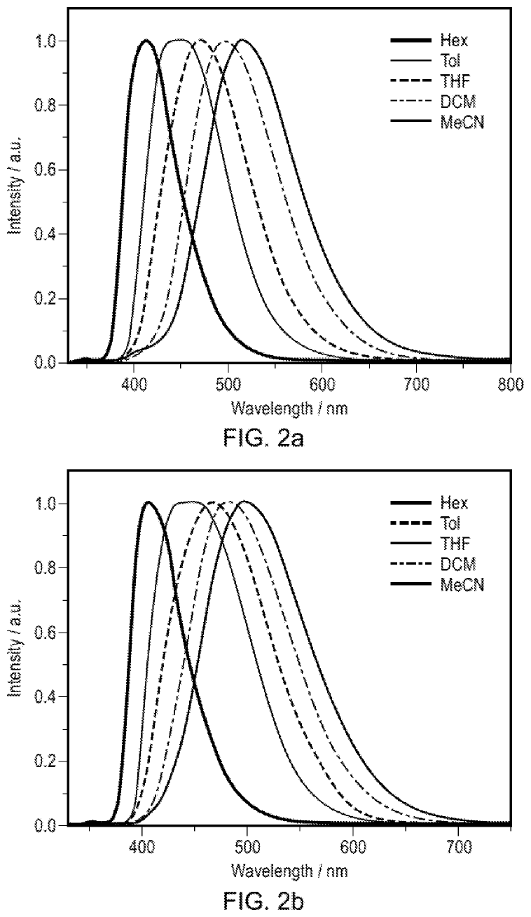

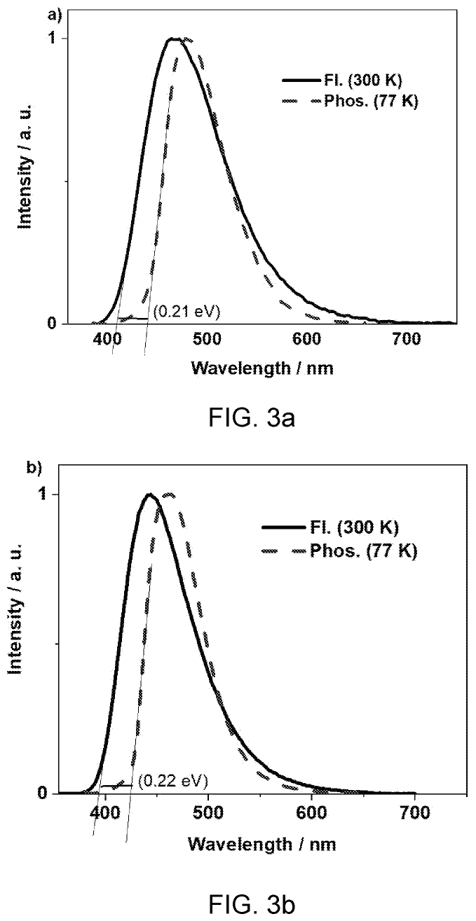

The ultraviolet-visible (UV-vis) absorption and steady-state photoluminescence (PL) spectra (in solution with various solvents) of XXVIII and XXIX are shown in FIGS. 1 and 2 respectively. Emission spectra are shown in hexane (Hex), toluene (Tol), tetrahydrofuran (THF), dichloromethane (DCM) and acetonitrile (MeCN). These materials show a strong intramolecular charge transfer (ICT) absorption band at 356 nm and 367 nm, respectively for pDTCz-2DPyS (also referred to as 2DPS-pDTCz or XXIX) and pDTCz-3DPyS (also referred to n as 3DPS-pDTCz or XXVIII). Emission spectra are broad and structureless in solution, and the emission spectra are bathochromically shifted in polar solvents, both indications of an emission from an ICT state. The emission maximum changes from 412 nm in hexane to 516 nm in MeCN for pDTCz-2DPyS (also referred to as 2DPS-pDTCz or XXIX) and 407 nm in hexane to 499 nm in MeCN for pDTCz-3DPyS (also referred to as 3DPS-pDTCz or XXVIII). Interestingly, the FWHM (peak Full Width at Half Maximum) of pDTCz-3DPyS (XXVIII) and pDTCz-2DPyS (XXIX) in hexane solution are 57 nm and 67 nm, respectively. These values are much smaller than a typical r TADF emitter. (A FWHM of 90 nm for the compound 2′-(phenoxazin-10-yl)-[1,1′:3′,1″-terphenyl]-5′-carbonitrile has been reported—ACS Appl. Mater. Interfaces, 2016, 8, 16791.) Herein, pDTCz-3DPyS (XXVIII) shows a narrower emission compared to pDTCz-2DPyS (XXIX) which is thought to be due to strong intramolecular H-bonding between pyridine and tert-butyl carbazole, which is not possible in pDTCz-2DPyS (XXIX). The photophysical properties in doped thin films in a suitably high triplet energy host, PPT, were investigated. In order to elucidate ΔE_{ST} the fluorescence and phosphorescence spectra were measured at 300 K and 77 K, respectively and the ΔE_{ST}, calculated from the difference between the onsets of these spectra, were found to be 0.21 and 0.22 eV for pDTCz-2DPyS and pDTCz-3DPyS, respectively.

TABLE 1
Photophysical properties of pDTCz-3DPyS (also referred to as 3DPS-pDTCz or XXVIII)
(first entry in Table) and pDTCz-2DPyS (also referred to as 2DPS-pDTCz or XXIX)
(second entry in table).

| | | | | HOMO/ | LUMO/ | E_{g}(S₁)/ | E_{T}(T₁)/ | ΔE_{ST}/ | |
|---|---|---|---|---|---|---|---|---|---|
| Emitter | λ_{abs}/nm^{a} | λ_{PL}/nm^{b} | λ_{PL}/nm^{c} | eV^{d} | eV^{e} | eV^{f} | eV^{g} | eV^{h} | Φ_{PL}/%^{I} |
| pDTCz-3DPyS | 367 | 444 | 462 | −5.71 | −2.57 | 3.14 | 2.92 | 0.22 | 62 |
| pDTCz-2DPyS | 356 | 467 | 478 | −5.75 | −2.73 | 3.02 | 2.81 | 0.21 | 67 |

^{a}ICT band measured in toluene at room temperature.
^{b}Fluorescence spectra measured in co-doped film at 300K in PPT host.
^{c}Phosphorescence spectra measured in a film with 7 wt % in PPT host at 77K.
^{d}Determined from the oxidation potential observed by CV in 10⁻³M DCM.
^{e}Calculated from HOMO + E_{g}.
^{f}E_{g} values are estimated from the onset of the fluorescence spectrum.
^{g}Estimated from the onset of phosphorescence spectrum.
^{h}λE_{ST} = E(S₁) − E(T₁).
^{I}Absolute Φ_{PL} of 7 wt % PPT film measured using an integrating sphere.

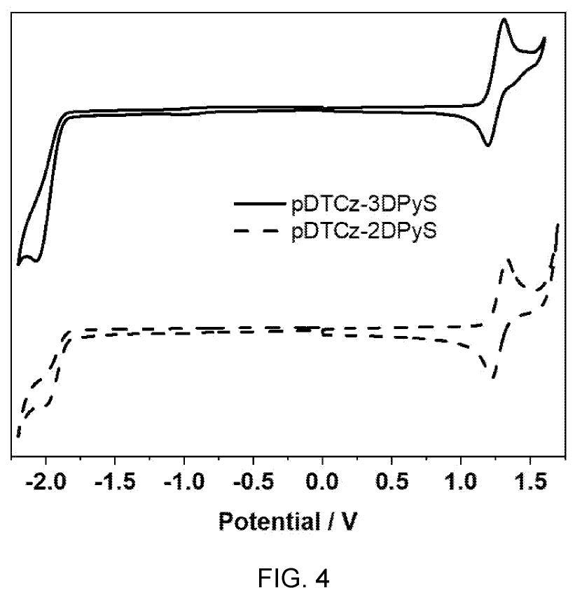

Electrochemical measurements on pDTCz-3DPyS (also referred to as 3DPS-pDTCz or XXVIII) and pDTCz-2DPyS (also referred to as 2DPS-pDTCz or XXIX) were carried out in DCM (dichloromethane). The cyclic voltammetry (CV) traces are shown in FIG. 4. The results are reported versus SCE (Standard Calomel Electrode) (Fc/Fc+=0.34 V in DCM). The oxidation waves were found to be reversible while reduction waves were found to be irreversible. The oxidation potential for XXIX [E_{1/2}^{(ox)}=1.29 V] and XXVIII [E_{1/2}^{(ox)}=1.25 V]. The HOMO levels −5.75 eV and −5.71 eV for pDTCz-2DPyS and pDTCz-3DPyS, respectively, were calculated from E_{1/2}^{(ox)} (E_{HOMO}=E_{1/2}^{(ox)}+4.8−0.34). The LUMO levels −2.73 and −2.57 eV, for pDTCz-2DPyS and pDTCz-3DPyS respectively, were estimated from the equation HOMO-E_{g} (Table 1), where the E_{g} is the singlet energy gap and determined from the onset of fluorescence spectrum.

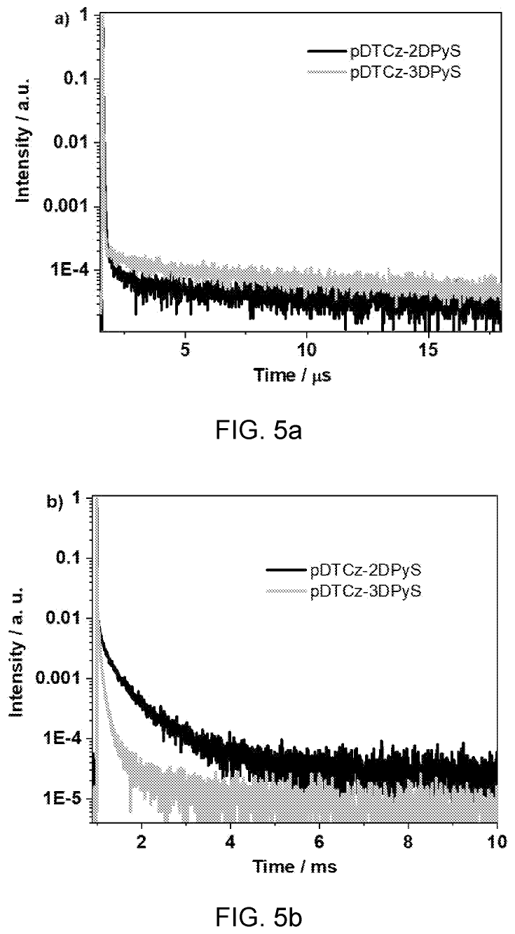

To confirm that these materials have TADF behaviour, the transient PL (photo luminescent) decay characteristic of these materials was measured in 10⁻⁵ M in toluene solution under vacuum and are shown in FIG. 5a. The transient decay curve of pDTCz-3DPyS (also referred to as 3DPS-pDTCz or XXVIII) shows biexponential decays with the prompt and delayed fluorescence lifetimes of 7.1 ns (95.43%) and 0.45 μs (4.57%), respectively. Similarly, the transient decay curve of pDTCz-2DPyS (also referred to as 2DPS-pDTCz or XXIX) also shows biexponential decay with the prompt and delayed fluorescence lifetimes of 15.1 ns (84.1%) and 0.85 μs (15.9%), respectively. The results support that these materials are TADF. To study the photophysical properties in the thin-film state, both materials were co-doped with the host matrix (PPT) in order to avoid concentration quenching. For pDTCz-3DPyS (XXVIII) the τₚ is 7.0 ns (98.8%) and the τ_{d} is 33.2 μs (1.2%) while for pDTCz-2DPyS (XXIX) the τₚ is 13.3 ns (73.8%) and the τ_{d} is biexponential in nature with 27.1 μs (16.4%) and 99.5 μs (9.8%). pDTCz-3DPySpDTCz-2DPyS

To further confirm the TADF mechanism, the Φ_{PL} was measured in a 7 wt % doped PPT film under a N₂ atmosphere. The Φ_{PL} was measured for pDTCz-2DPyS (XXIX), pDTCz-3DPyS (XXVIII) and pDTCz-DPS (reference compound, see below).

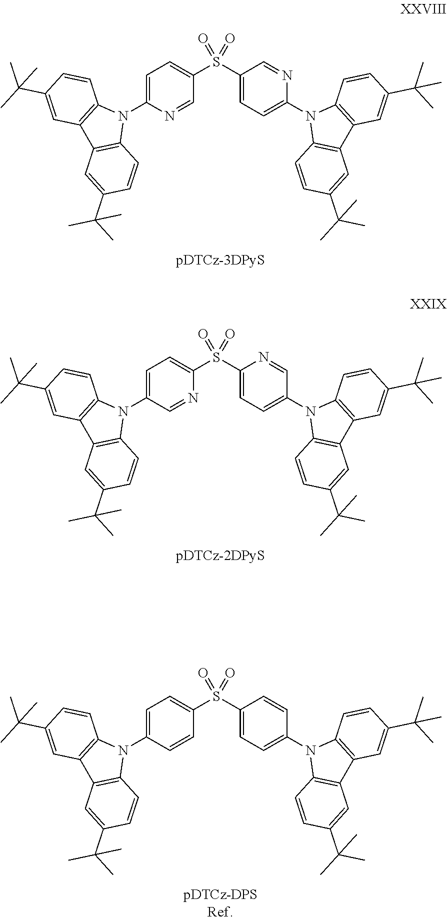

The DPL values measured were 67%, 62%, and 60%, respectively, for pDTCz-2DPyS (XXIX), pDTCz-3DPyS (XXVIII) and pDTCz-DPS (ref). The Φ_{PL} values decreased to 55%, 49% and 59%, respectively, under air, indicating the presence of an accessible triplet state. This observation further confirms that these materials are TADF emitters.

### 9-(5-bromopyrazin-2-yl)-3,6-di-tert-butyl-9H-carbazole (Br-Pz-tCz)

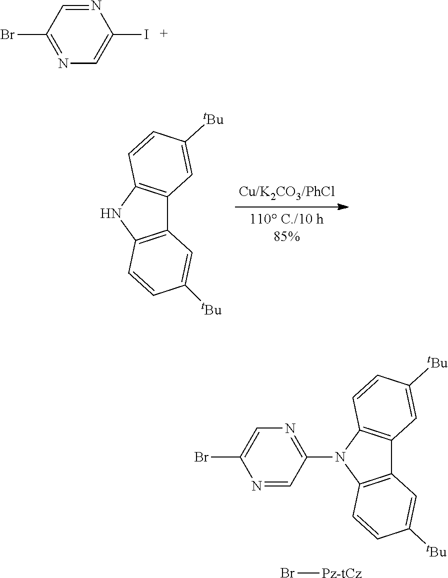

To a 250 mL flask were added 2-bromo-5-iodopyrazine (2.9 g, 10 mmol, 1 equiv.), di-tert-butyl-9H-carbazole (3.0 g, 11 mmol, 1.1 equiv.), copper powder (640 mg, 10 mmol, 1 equiv.) and potassium carbonate (4.5 g, 30 mmol, 3 equiv.). The flask was degassed by three cycles of vacuum-nitrogen purging and 50 mL of chlorobenzene was injected. The mixture was stirred at 110° C. for 10 h under a nitrogen atmosphere. After cooling, water was added to the reaction mixture followed by extraction with DCM (3×50 mL). The combined organic layers were dried with anhydrous magnesium sulfate. The organic solvent was removed under reduced pressure and the crude product was purified by silica gel column chromatography. DCM/Hexane=1/3 was used as eluent to afford Br-Pz-tCz as a faint yellow solid. Yield: 85%. R_{f}: 0.65 (33% DCM/Hexanes). Mp: 180-182° C. ¹H NMR (400 MHz, CDCl₃) δ (ppm): 8.84 (d, J=1.4 Hz, 1H), 8.74 (d, J=1.4 Hz, 1H), 8.13 (dd, J=2.0, 0.6 Hz, 2H), 7.84 (dd, J=8.8, 0.7 Hz, 2H), 7.54 (dd, J=8.7, 2.0 Hz, 2H), 1.49 (s, 18H). ¹³C NMR (101 MHz, CDCl₃) δ (ppm): 147.97, 145.94, 145.28, 139.30, 137.06, 134.57, 125.10, 124.37, 116.46, 110.73, 34.85, 31.87. HR-MS (LTQ Orbitrap XL) [M+H]⁺ Calculated: (C₂₄H₂₇N₃Br) 436.1383, 438.1363; Found: 436.1380, 438.1359.

### bis(5-(3,6-di-tert-butyl-9H-carbazol-9-yl)pyrazin-2-yl)sulfane (tCz-PzS)

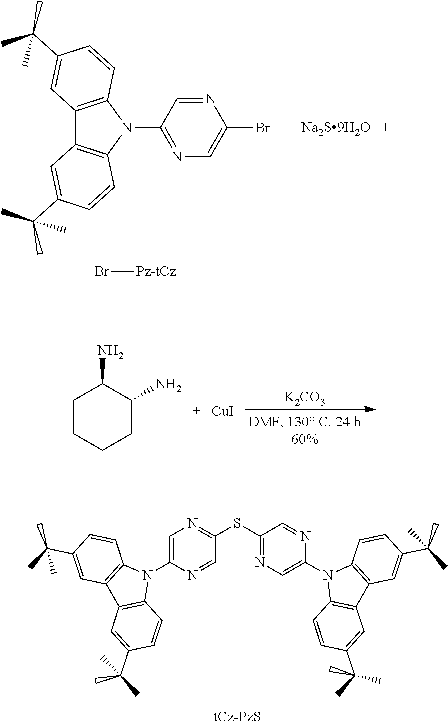

To a 100 mL three neck flask were added Br-Pz-tCz (960 mg, 2.2 mmol, 2.2 equiv.), sodium sulfide hydrate (100 mg, 1 mmol, 1 equiv.), copper(I) iodide (40 mg, 0.2 mmol, 0.2 equiv.), trans-1,2-cyclohexanediamine (45 mg, 0.4 mmol, 0.4 equiv.), and potassium carbonate (700 mg, 5 mmol, 5 equiv.). The flask was degassed by three cycles of vacuum-nitrogen purging and 20 mL of DMF was injected. The mixture was stirred at 130° C. for 24 h under a nitrogen atmosphere. The reaction mixture poured into 100 mL of icy water and extracted by ethyl acetate (3×20 mL). The combined organic layers were dried with magnesium sulfate and organic solvent was removed under reduced pressure. The crude product was purified by silica gel column chromatography. DCM/Hexane=1/1 was used as eluent to afford tCz-PzS as light-yellow solid. Yield: 60%. R_{f}: 0.52 (33% DCM/Hexanes). Mp: 211-212° C. ¹H NMR (500 MHz, CDCl₃) δ (ppm): 9.02 (s, 2H), 8.85 (s, 2H), 8.13 (d, J=1.9 Hz, 4H), 7.91 (d, J=8.7 Hz, 4H), 7.55 (dd, J=8.7, 1.9 Hz, 4H), 1.49 (s, 36H). ¹³C NMR (125 MHz, CDCl₃) δ (ppm): 151.27, 148.34, 145.30, 144.81, 140.61, 137.51, 136.98, 125.16, 124.37, 116.46, 110.83, 34.83, 31.88. HR-MS (LTQ Orbitrap XL) [M+H]⁺ Calculated: (C₄₈H₅₃N₆S) 745.4047, 746.4078; Found: 745.4043, 746.4077.

### 9,9′-(sulfonylbis(pyrazine-5,2-diyl))bis(3,6-di-tert-butyl-9H-carbazole) (pDTCz-DPzS)

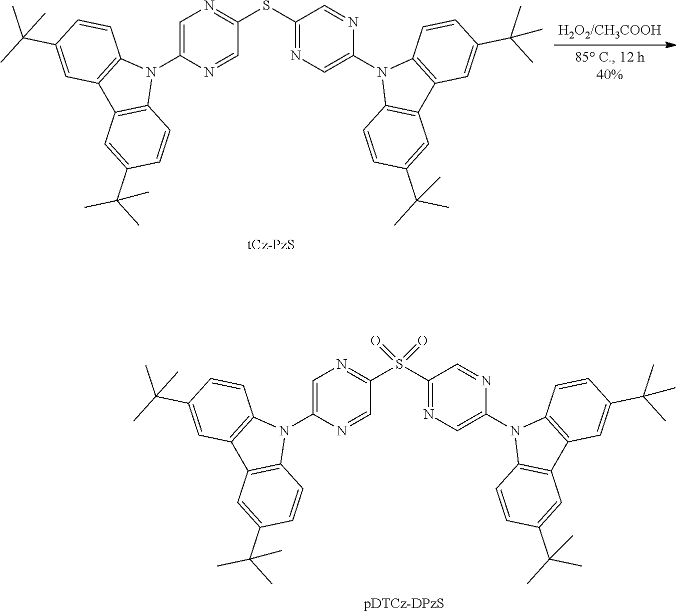

To a 100 mL two neck flask were added tCz-PzS (1.5 g, 2 mmol, 1 equiv.) and 20 mL of acetic acid. After tCz-PzS was dissolved in acetic acid, 30 mL of 30 wt % hydrogen peroxide was injected. The mixture was heated to 85° C. for 12 h. The mixture was then poured into 100 mL of icy water and extracted by DCM (3×50 mL). The combined organic layers were dried with magnesium sulfate and the organic solvent was removed under reduced pressure. The crude product was purified by silica gel column chromatography. DCM/Hexanes=4/1 was used as eluent to afford pDTCz-DPzS as a light-green solid. Yield: 40%. R_{f}: 0.68 (75% DCM/Hexanes). Mp: 292-294° C. ¹H NMR (500 MHz, CDCl₃) δ (ppm): 9.54 (d, J=1.3 Hz, 2H), 9.18 (d, J=1.3 Hz, 2H), 8.10 (d, J=1.9 Hz, 4H), 8.06 (d, J=8.7 Hz, 4H), 7.55 (dd, J=8.8, 2.0 Hz, 4H), 1.49 (s, 36H). ¹³C NMR (126 MHz, CDCl₃) δ (ppm): 151.54, 146.72, 145.74, 144.11, 138.26, 136.60, 126.09, 124.70, 116.63, 112.09, 34.91, 31.79. HR-MS (LTQ Orbitrap XL) [M+H]⁺ Calculated: (C₄₈H₅₃N₆O₂S) 777.3945, 778.3976; Found: 777.3939, 778.3972. Elemental analysis: Calcd for C₄₈H₅₂N₆O₂S: C, 74.20; H, 6.75; N, 10.82. Found: C, 74.38; H, 6.81; N, 10.95. HPLC: 10% H₂O/MeCN, 1.0 mL min-, 300 nm; tr (98.5%)=17.7 min.

### 9-(5-bromopyrimidin-2-yl)-3,6-di-tert-butyl-9H-carbazole (Br—Pm-tCz)

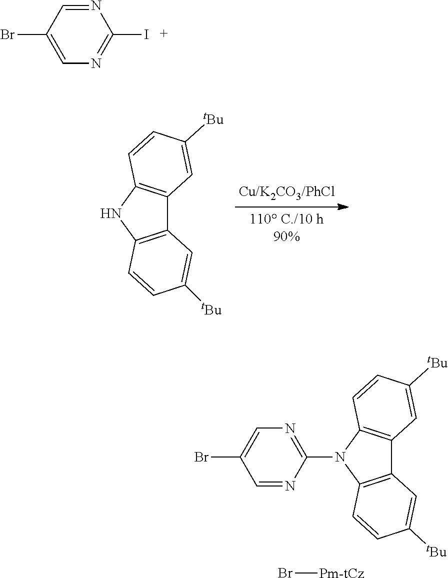

To a 250 mL flask were added 5-bromo-2-iodopyrimidine (2.9 g, 10 mmol, 1 equiv.), di-tert-butyl-9H-carbazole (3 g, 11 mmol, 1.1 equiv.), copper powder (640 mg, 10 mmol, 1 equiv.) and potassium carbonate (4.5 g, 30 mmol, 3 equiv.). The flask was degassed by three cycles of vacuum-nitrogen purging and 50 mL of chlorobenzene was injected. The mixture was stirred at 110° C. for 10 h under nitrogen atmosphere. The reaction mixture was then poured into water and extracted with DCM (3×50 mL). The combined organic layers were dried with anhydrous magnesium sulfate, filtered and the solvent removed under reduced pressure. The crude product was purified by silica gel column chromatography. DCM/Hexane=1/3 was used as eluent to afford Br—Pm-tCz as a white solid. Yield: 90%. R_{f}: 0.65 (33% DCM/Hexanes). Mp: 197-199° C. ¹H NMR (400 MHz, CDCl₃) δ (ppm): 8.81 (s, 2H), 8.72 (d, J=8.9 Hz, 2H), 8.08 (dd, J=2.0, 1.0 Hz, 2H), 7.57 (dd, J=8.8, 2.1 Hz, 2H), 1.50 (s, 18H). ¹³C NMR (101 MHz, CDCl₃) δ (ppm): 158.14, 157.29, 145.68, 137.23, 126.06, 124.33, 116.05, 115.62, 112.22, 34.77, 31.84. HR-MS (LTQ Orbitrap XL) [M+H]⁺ Calculated: (C₂₄H₂₇N₃Br) 436.1383, 438.1363; Found: 436.1380, 438.1359.

### bis(2-(3,6-di-tert-butyl-9H-carbazol-9-yl) pyrimidin-5-yl) sulfane (tCz-PmS)

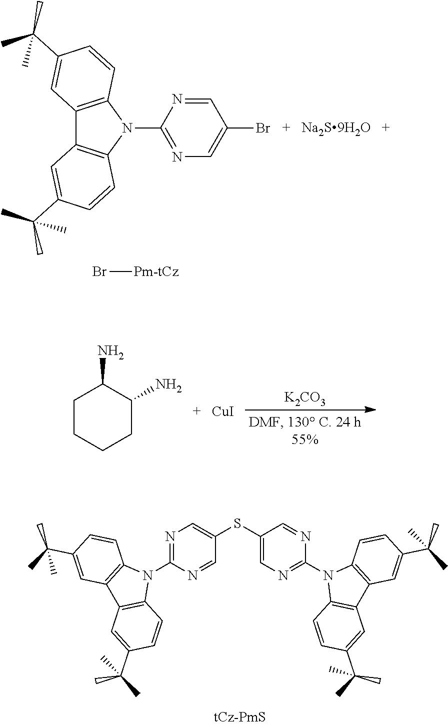

To a 100 mL three neck flask were added Br—Pm-tCz (960 mg, 2.2 mmol, 2.2 equiv.), sodium sulfide hydrate (100 mg, 1 mmol, 1 equiv.), copper(I) iodide (40 mg, 0.2 mmol, 0.2 equiv.), trans-1,2-cyclohexanediamine (45 mg, 0.4 mmol, 0.4 equiv.), and potassium carbonate (700 mg, 5 mmol, 5 equiv.). The flask was degassed by three cycles of vacuum-nitrogen purging and 20 mL of DMF was injected. The mixture was stirred at 130° C. for 24 h under a nitrogen atmosphere. The reaction mixture was poured into 100 mL of icy water and extracted with ethyl acetate (3×20 mL). The combined organic layers were dried with magnesium sulfate and the organic solvent was removed under reduced pressure. The crude product was purified by silica gel column chromatography. DCM/Hexane=1/1 was used as eluent to afford tCz-PmS as a light-yellow solid. Yield: 60%. R_{f}: 0.52 (33% DCM/Hexanes). Mp: 284-285° C. ¹H NMR (400 MHz, CDCl₃) δ (ppm): 8.92 (dd, J=7.8, 2.9 Hz, 2H), 8.72 (dd, J=8.9, 1.8 Hz, 2H), 8.08 (d, J=2.0 Hz, 2H), 7.57 (dd, J=8.9, 1.8 Hz, 2H), 1.50 (s, 18H). ¹³C NMR (101 MHz, CDCl₃) δ (ppm): 162.77, 158.04, 157.29, 145.68, 137.19, 126.11, 124.32, 116.19, 115.62, 34.77, 31.84. HR-MS (LTQ Orbitrap XL) [M+H]⁺ Calculated: (C₄₈H₅₃N₆S) 745.4047, 746.4078; Found: 745.4043, 746.4077.

### 9,9′-(sulfonylbis(pyrimidine-5,2-diyl))bis(3,6-di-tert-butyl-9H-carbazole) (pDTCz-DPmS)

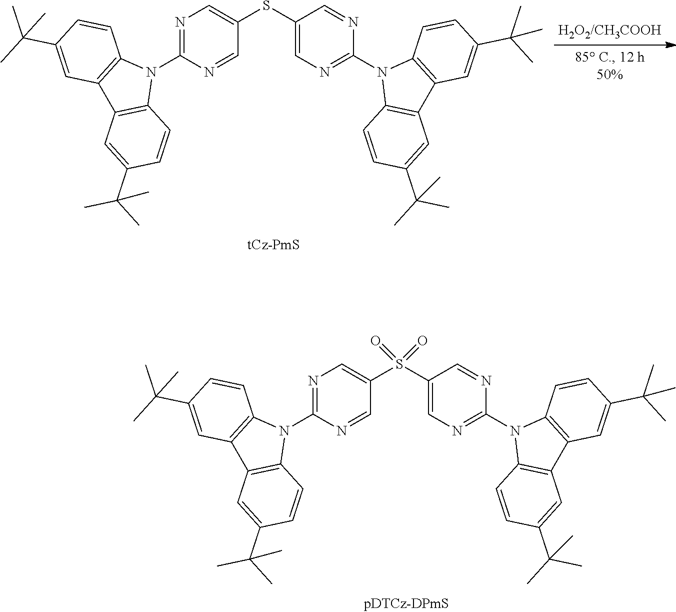

To a 100 mL of two neck flask were added tCz-PmS (1.5 g, 2 mmol, 1 equiv) and 20 mL of acetic acid. After tCz-PmS was dissolved in acetic acid, 30 mL of 30 wt % hydrogen peroxide were injected. The mixture was heated to 85° C. for 12 h. The mixture was then poured into 100 mL of icy water and extracted with DCM (3×50 mL). The combined organic layers were dried with magnesium sulfate, filtered and the organic solvent was removed under reduced pressure. The crude product was purified by silica gel column chromatography. DCM/Hexanes=4/1 was used as eluent to afford pDTCz-DPmS as white solid. Yield: 50%. R_{f}: 0.68 (75% DCM/Hexanes). Mp: 292-294° C. ¹H NMR (500 MHz, CDCl₃) δ (ppm): 9.31 (s, 4H), 8.84 (d, J=8.9 Hz, 4H), 8.04 (d, J=2.0 Hz, 4H), 7.57 (dd, J=8.9, 2.0 Hz, 4H), 1.48 (s, 36H). ¹³C NMR (126 MHz, CDCl₃) δ (ppm): 160.24, 157.35, 147.18, 137.06, 128.65, 127.05, 124.69, 117.57, 115.80, 34.83, 31.74. HR-MS (LTQ Orbitrap XL) [M+H]⁺ Calculated: (C₄₈H₅₃N₆O₂S) 777.3945, 778.3976; Found: 777.3942, 778.3974. Elemental analysis: Calcd for C₄₈H₅₂N₆O₂S: C, 74.20; H, 6.75; N, 10.82. Found: C, 74.10; H, 6.82; N, 10.82. HPLC: 10% H₂O/MeCN, 1.0 mL min⁻¹, 300 nm; tr (97.5%)=29.2 min.

Synthesis of Phosphinate Based Compounds

### Synthesis of I₂Cz

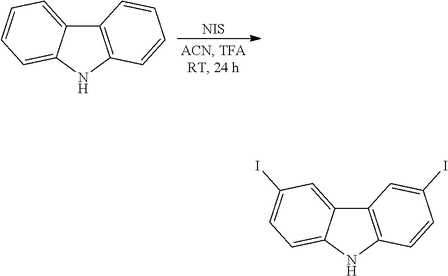

Carbazole (5.0 g, 30.0 mmol) was added to acetonitrile (200 mL) at room temperature. TFA (1 pipette full), followed by NIS (14.2 g, 63.0 mmol) was added over 20 minutes and the reaction was left to stir at room temperature covered in aluminium foil for 24 hr. Upon completion water was added and the resulting precipitate filtered. This crude product was then directly protected before further purification. ¹H NMR (400 MHz, CDCl₃): δ 8.35 (d, J=1.7 Hz, 2H), 7.71 (dd, J=8.5, 1.7 Hz, 2H), 7.24 (dd, J=8.5, 0.4 Hz, 2H).

### Synthesis of I₂CzTBDMS

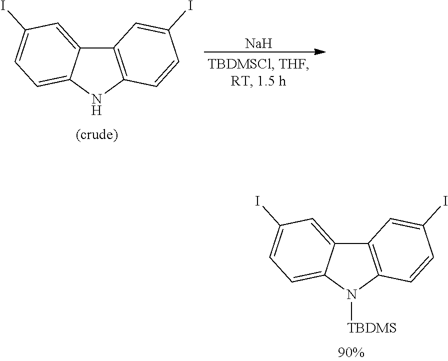

3,6-diiodo-9H-carbazole (5.00 g, 11.9 mmol) was dissolved in dry THF (50 mL) and the reaction vessel cycled 3 times with vacuum and nitrogen. 60% NaH in oil (0.95 g, 23.8 mmol) was added and the reaction left for 30 minutes. Tert-butyldimethylsilylchloride (2.16 g, 14.3 mmol) was then added and the solution left to stir for 1 hr. Upon completion, the reaction was quenched with distilled water and ethyl acetate was used for extraction. Purification of the crude was performed using column chromatography using EtOAc:hexane (3:97) affording a slightly off-white powder (5.71 g, 90%). ¹H NMR (400 MHz, CDCl₃): δ 8.33 (d, J=1.8 Hz, 2H), 7.64 (dd, J=8.8, 1.9 Hz, 2H), 7.38 (d, J=8.8 Hz, 2H), 1.03 (s, 9H), 0.75 (s, 6H).

### Synthesis of 3CzTBDMS

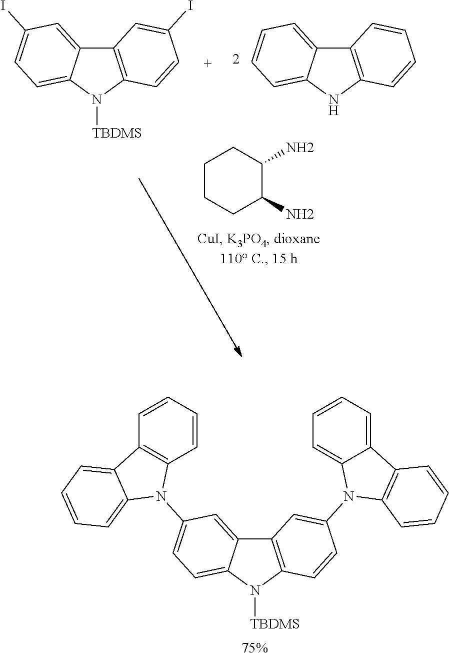

9H-carbazole (1.24 g, 7.42 mmol), 9-tert-butyldimethylsilyl-3,6-diiodocarbazole (1.93 g, 3.62 mmol), CuI (0.07 g, 0.36 mmol) and K₃PO₄ (4.66 g, 21.94 mmol) were added to a 2-necked flask equipped with a condenser and cycled 3 times with vacuum and nitrogen. (±)-trans-1,2-cyclohexanediamine (0.06 g, 0.54 mmol) and dried 1,4-dioxane (30 mL) were added and the reaction left to stir at 110° C. under the flow of nitrogen for 25 hours. Once complete, the reaction was allowed to cool, diluted with toluene, filtered through silica and dried. Purification was performed using column chromatography EtOAc:hexane (2:98) affording an off-white powder (3.41 g, 75%). ¹H NMR (400 MHz, CDCl₃): δ 8.21 (d, J=2.1 Hz, 2H), 8.18 (dt, J=7.8, 1.0 Hz, 4H), 7.90-7.86 (m, 2H), 7.59 (dd, J=8.8, 2.2 Hz, 2H), 7.45-7.38 (m, 8H), 7.32-7.27 (m, 4H), 1.22 (d, J=1.1 Hz, 9H), 0.93 (s, 6H).

### Synthesis of 3tCzTBDMS

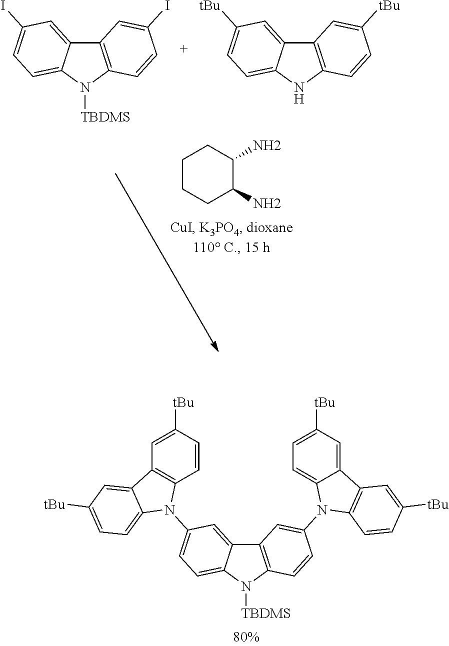

Synthesis procedure of 3CzTBDMS is followed to synthesize 3tCzTBDMS. Crude product was recrystallized from ethanol to get pure compound (80%). ¹H NMR (400 MHz, CDCl₃): δ 8.17 (dt, J=1.8, 0.8 Hz, 6H), 7.84 (d, J=8.8 Hz, 2H), 7.57 (dd, J=8.8, 2.2 Hz, 2H), 7.46 (dd, J=8.7, 2.0 Hz, 4H), 7.36 (dd, J=8.6, 0.6 Hz, 4H), 1.48 (s, 36H), 1.21 (s, 9H), 0.91 (s, 6H).

### Synthesis of 3Cz

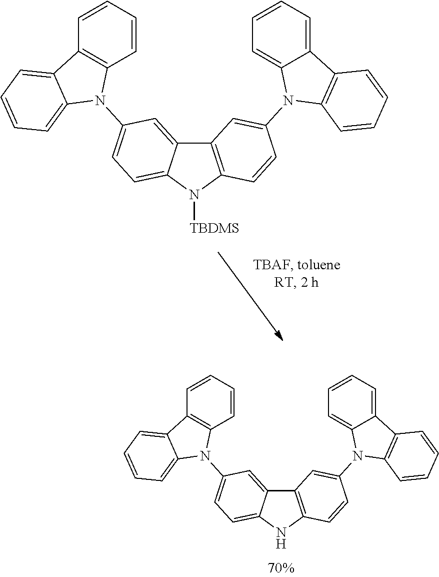

3CzTBDMS (1.65 g, 2.7 mmol), TBAF.3H₂O (1.26 g, 4.0 mmol) and toluene (15 mL) were combined and allowed to stir for 2 hr at room temperature. Upon completion, the reaction mixture was quenched with sat. NH₄Cl and extracted with DCM. DCM was evaporated off and the solid was recrystallized from ethanol (70%). ¹H NMR (400 MHz, CDCl₃): δ 9.69 (s, 1H), 8.21 (s, 2H), 8.18 (d, J=7.8 Hz, 4H), 7.83 (d, J=8.6 Hz, 2H), 7.62 (d, J=8.6 Hz, 2H), 7.42-7.37 (m, 8H), 7.30 (dd, J=6.0, 1.9 Hz, 3H), 7.28-7.27 (m, 1H). HRMS (m/z): [M+H]⁺ calculated for C₃₆H₂₃N₃: 498.1965, found: 498.1958.

### Synthesis of t3Cz

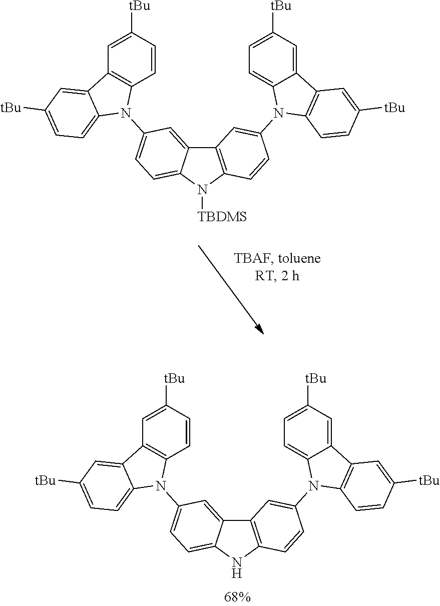

Deprotection and subsequent purification as above (3Cz), resulting in a white solid (68%). ¹H NMR (400 MHz, CDCl₃): δ 8.43 (s, 1H), 8.18 (dd, J=1.9, 0.7 Hz, 4H), 7.66-7.60 (m, 2H), 7.51-7.45 (m, 4H), 7.40-7.27 (m, 2H), 7.27-7.26 (m, 1H), 7.23-7.15 (m, 5H), 1.49 (s, 36H). HRMS (m/z): [M+H]⁺ calculated for C₅₂H₅₅N₃: 722.4469, found: 722.4463.

### Synthesis of 3CzPyBr

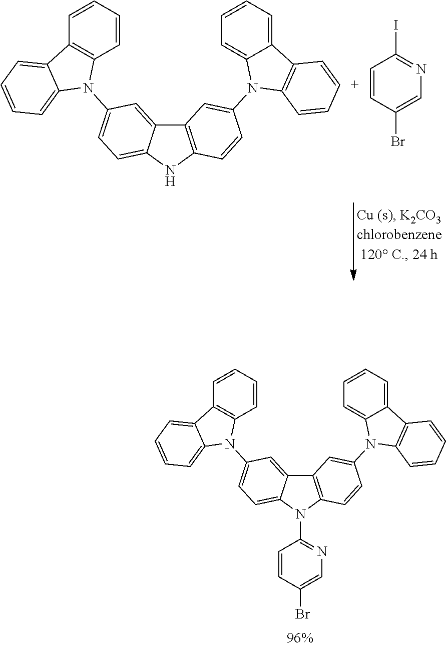

3Cz (0.50 g, 1.0 mmol), 5-bromo-2-iodopyridine (0.29 g, 1.0 mmol), copper (0.07 g, 1.0 mmol) and K₂CO₃ (0.28 g, 2.0 mmol) were combined in a 2-necked flask equipped with a condenser and cycled 3 times with vacuum and nitrogen. Chlorobenzene (15 mL) was then added and stirred at 120° C. under the flow of nitrogen for 19 hours whereupon full consumption of both starting materials was observed. The reaction was then allowed to cool and the copper filtered out over Celite bed and washed with DCM. The solvents were removed under vacuum resulting in an off-white solid (0.63 g, 96%). ¹H NMR (400 MHz, CDCl₃): δ 8.89 (dd, J=2.5, 0.6 Hz, 1H), 8.29 (d, J=1.7 Hz, 2H), 8.20 (d, J=7.7 Hz, 3H), 8.12 (dd, J=8.5, 2.5 Hz, 1H), 8.09 (d, J=8.7 Hz, 2H), 7.72-7.67 (m, 2H), 7.46-7.42 (m, 6H), 7.35-7.30 (m, 4H). HRMS (m/z): [M+H]⁺ calculated for C₄₁H₂₅BrN₄: 653.1335, found: 653.1331.

### Synthesis of 3tCzPyBr

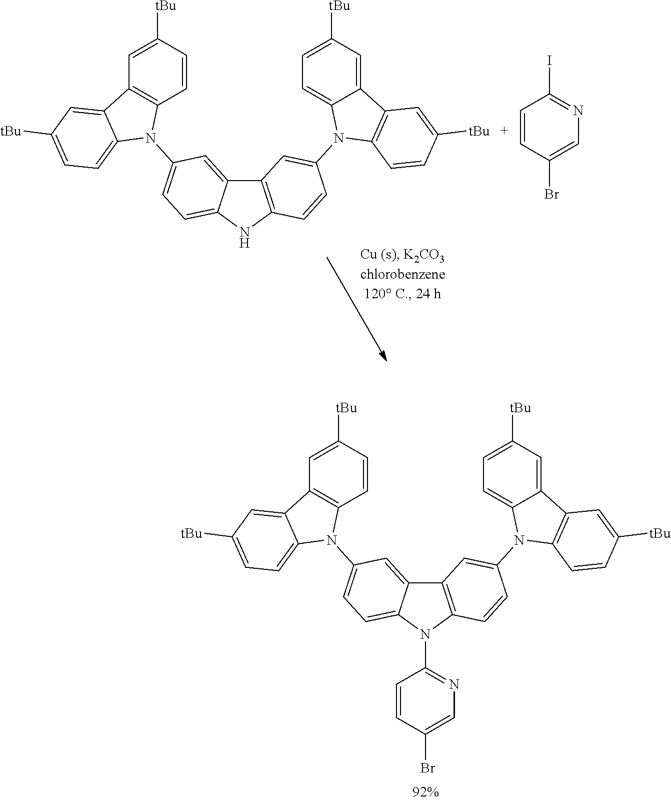

Procedure as above with t3CzPyBr, resulting in an off-white powder (92%). ¹H NMR (400 MHz, CDCl₃): δ 8.90 (d, J=2.5 Hz, 1H), 8.24 (d, J=2.0 Hz, 2H), 8.21-8.16 (m, 5H), 8.08 (d, J=8.7 Hz, 2H), 7.77 (d, J=8.5 Hz, 1H), 7.69 (dd, J=8.8, 2.1 Hz, 2H), 7.49 (dd, J=8.7, 1.9 Hz, 4H), 7.37 (dd, J=8.6, 0.6 Hz, 4H), 1.49 (s, 36H).

### Synthesis of 3tCzPzBr

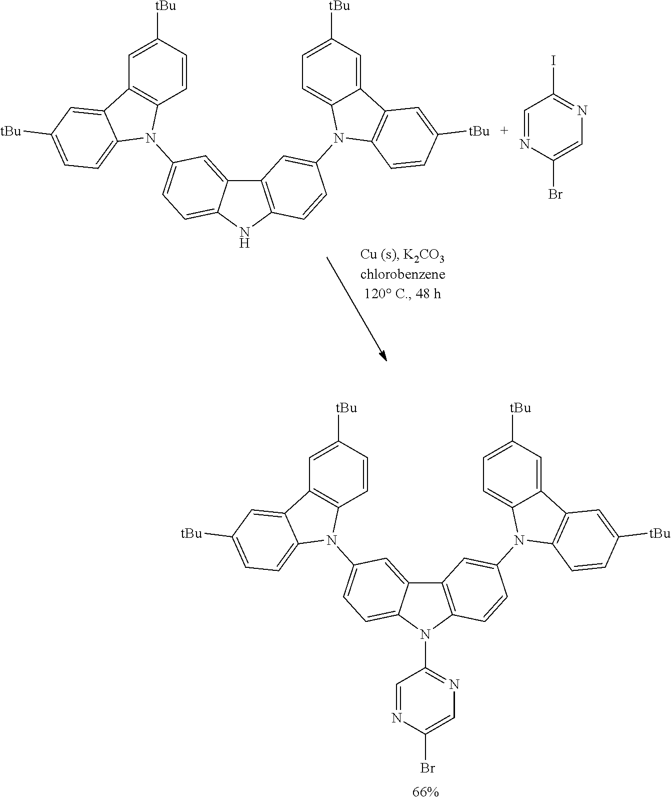

Procedure as with previous pyridine coupling (3CzPyBr) was followed. After 24 hours TLC indicated large presence of remaining t3Cz, it was observed that the 120° C. was not achieved and thus an alternate side-reaction may have occurred. Additional 5-bromo-2-iodopyrazine (1 eq) and K₂CO₃ (2 eq) were added and the temperature increased to 130° C. and the reaction left for a further 24 hours. Improvement was seen so the reaction mixture was filtered over Celite bed and washed with DCM, affording a yellow powder (66%). ¹H NMR (400 MHz, CDCl₃): δ 9.02 (d, J=1.4 Hz, 1H), 8.89 (d, J=1.4 Hz, 1H), 8.26 (dt, J=2.4, 1.2 Hz, 2H), 8.19 (dd, J=2.0, 0.6 Hz, 4H), 8.15-8.10 (m, 2H), 7.73 (dd, J=8.7, 2.0 Hz, 2H), 7.49 (dd, J=8.6, 1.9 Hz, 4H), 7.37 (dd, J=8.6, 0.7 Hz, 4H), 1.49 (s, 36H). HRMS (m/z): [M+H]⁺ calculated for C₅₆H₅₆BrN₅: 878.3792, found: 878.3788.

### Synthesis of 3CzPyPO (XXXIII)

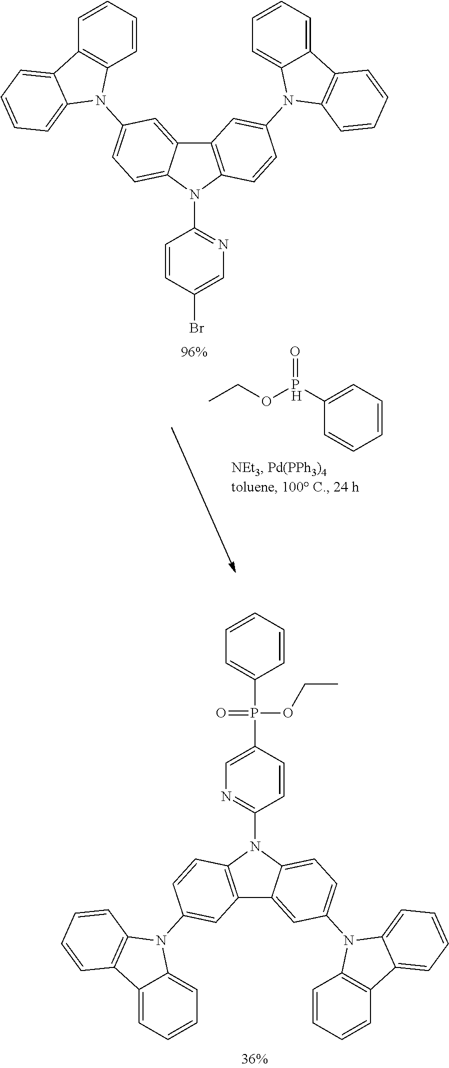

3CzPyBr (0.50 g, 0.77 mmol), Pd(PPh₃)₄ (0.09 g, 0.08 mmol) and ethyl phenylphosphinate (0.13 g, 0.77 mmol) are combined in a flask and cycled 3 times with vacuum and nitrogen. Finally n-methylmorpholine (0.16 g, 1.53 mmol) and dry toluene (15 mL) were added without further vacuum cycling due to the base volatility. The reaction was then left to stir at 100° C. under the flow of nitrogen for 24 hours. Monitoring of the reaction via TLC after 24 hours showed no further conversion of starting material to product so the reaction was cooled and filtered using Celite bed with DCM and MeOH. An NMR of the crude at this point indicated a 60% product conversion via the integration of the downfield pyridine peak. Purification was performed using column chromatography with an initial eluent of 5% EtOAc:Hex which was increased incrementally to 50% in order to remove remaining starting material and multiple less polar impurities present. In order to wash the product an eluent of 1% MeOH:DCM was used, resulting in a pale yellow solid (0.21 g, 36%). ¹H NMR (400 MHz, CDCl₃): δ 9.20 (dd, J=5.8, 1.6 Hz, 1H), 8.46 (ddd, J=11.1, 8.3, 2.2 Hz, 1H), 8.29 (d, J=1.8 Hz, 2H), 8.23 (d, J=8.8 Hz, 2H), 8.19 (d, J=7.7 Hz, 4H), 8.04-7.97 (m, 2H), 7.95-7.91 (m, 1H), 7.70 (dd, J=8.8, 2.1 Hz, 2H), 7.66 (dd, J=7.3, 1.4 Hz, 1H), 7.62 (dd, J=7.4, 3.6 Hz, 2H), 7.43 (d, J=3.7 Hz, 8H), 7.32 (dt, J=8.1, 4.1 Hz, 4H), 4.40-4.22 (m, 2H), 1.53 (t, J=7.1 Hz, 3H); ¹³C{¹H} NMR (126 MHz, CDCl₃): δ 153.9, 152.7, 152.6, 142.2, 142.1, 141.5, 138.7, 132.9, 132.9, 131.9, 131.9, 131.8, 129.1, 129.0, 126.6, 126.0, 125.6, 123.2, 120.4, 119.9, 119.6, 118.0, 117.9, 113.2, 109.6, 61.8, 16.7; ³¹P{¹H} NMR (202 MHz, CDCl₃): δ 28.27. HRMS (m/z): [M+H]⁺ calculated for C₄₉H₃₅N₄O₂P: 743.2531, found: 743.2572.

### Synthesis of t3CzPyPO (XXXIV)

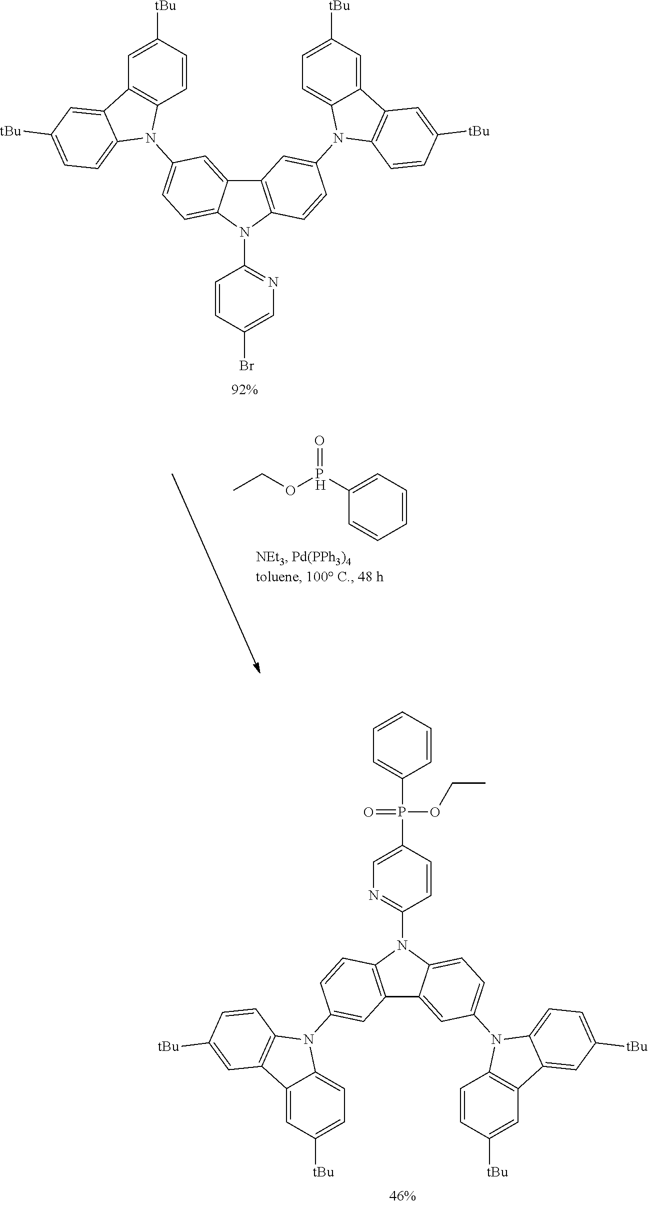

Procedure of 3CzPyPO followed. Reaction was left for 48 hours and crude product was recrystallized from hexane and a pale yellow powder (46%). ¹H NMR (400 MHz, CDCl₃): δ 9.20-9.15 (m, 1H), 8.49-8.41 (m, 1H), 8.25-8.16 (m, 8H), 8.03-7.92 (m, 2H), 7.75-7.68 (m, 4H), 7.63-7.56 (m, 2H), 7.50-7.46 (m, 5H), 7.39-7.35 (m, 3H), 4.38-4.21 (m, 2H), 1.56-1.46 (m, 39H); ¹³C {¹H} NMR (126 MHz, CDCl₃): δ 154.0, 152.6, 152.5, 142.7, 142.2, 142.1, 139.9, 138.4, 135.2, 132.9, 132.5, 131.9, 131.8, 129.1, 129.0, 126.3, 125.6, 123.7, 123.2, 119.1, 117.9, 117.9, 116.3, 113.1, 109.0, 61.8, 34.8, 32.1, 16.6; ³¹P{¹H} NMR (202 MHz, CDCl₃): δ 28.33. HRMS (m/z): [M+H]⁺ calculated for C₆₅H₆₇N₄O₂P: 967.5002, found: 967.5064

### Synthesis of t3CzPzPO (XXXVI)

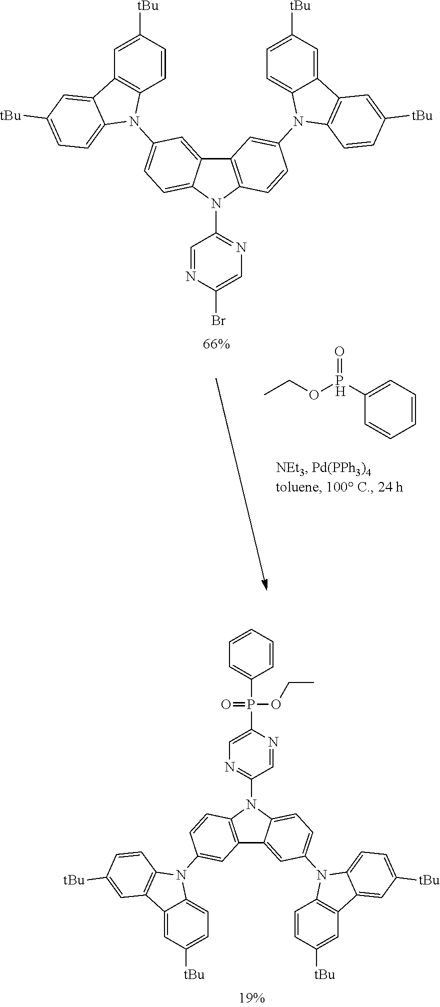

Procedure of 3CzPyPO followed. TLC after 24 hours indicated the occurrence of multiple side reactions. Purification was done by a dry column chromatography with increasing polarity. This afforded a glassy yellow solid (19%). ¹H NMR (400 MHz, CDCl₃): δ 9.44 (s, 1H), 9.41 (s, 1H), 8.32-8.28 (m, 4H), 8.22 (d, J=2.0 Hz, 4H), 8.19-8.12 (m, 2H), 7.79-7.73 (m, 2H), 7.69-7.59 (m, 2H), 7.52 (dd, J=8.7, 2.0 Hz, 4H), 7.46-7.37 (m 5H), 4.17 (q, J=7.1 Hz, 2H), 1.57-1.50 (m, 39H)¹³C{¹H} NMR (126 MHz, CDCl₃): δ 149.6, 147.4, 147.2, 146.7, 145.4, 142.9, 140.1, 140.0, 139.7, 137.9, 133.3, 133.1, 132.5, 132.5, 132.2, 132.2, 129.8, 128.9, 128.8, 126.5, 126.2, 123.7, 123.3, 119.2, 116.3, 113.2, 109.0, 62.2, 34.8, 32.1, 16.7; 31P{¹H} NMR (202 MHz, CDCl₃): δ 24.08; HRMS (m/z): [M+H]⁺ calculated for C₆₄H₆₆N₅O₂P: 968.5027, found: 968.5023.

Photophysical Properties of Phosphorus Containing Compounds

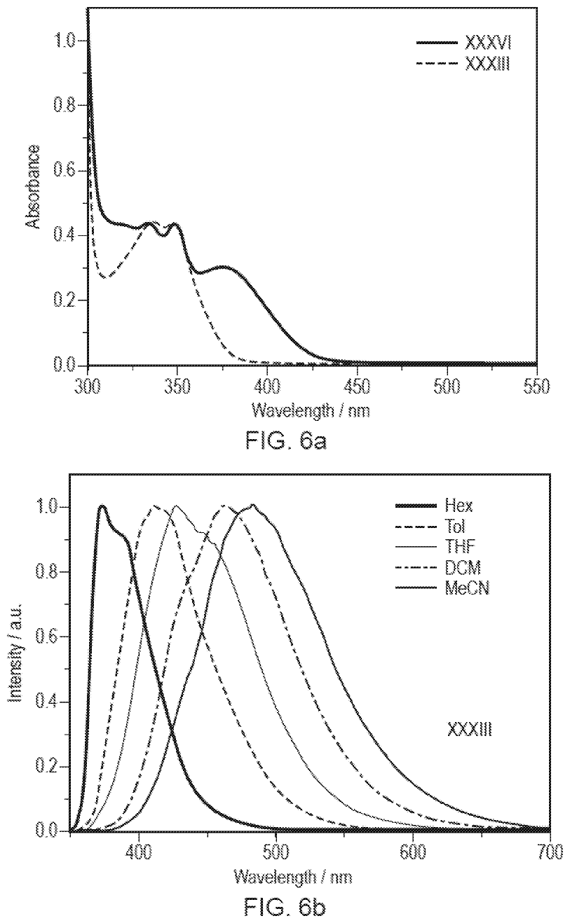

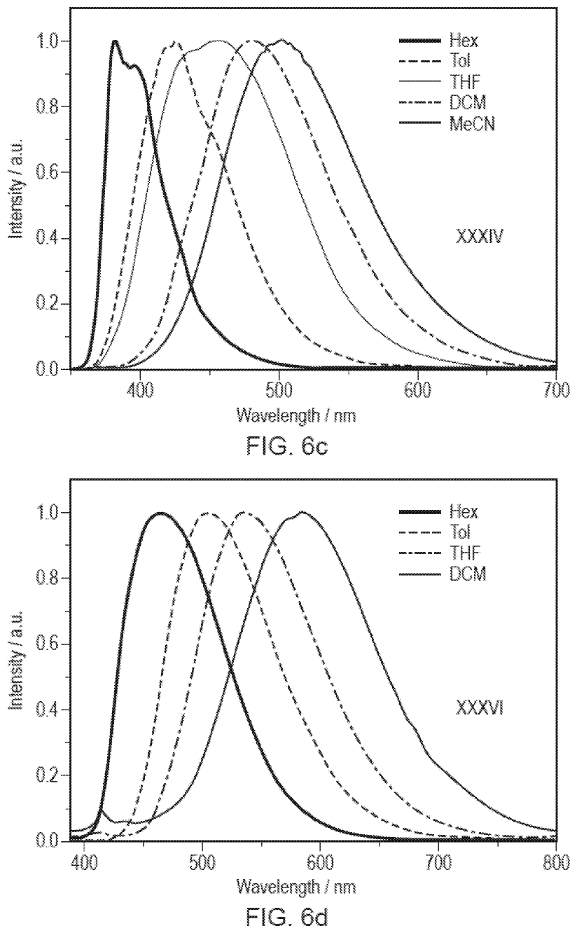

The ultraviolet-visible (UV-vis) absorption of t3CzPyPO (XXXIII) and t3CzPzPO (XXXVI) and steady-state photoluminescence (PL) spectra of 3CzPyPO (XXXIII), t3CzPyPO (XXXIV) and t3CzPzPO (XXXVI) and are shown in FIGS. 6a to 6d. The (UV-vis) absorption spectrum in FIG. 6a for t3CzPyPO shows a structured absorption at 336 and 348 nm and t3CzPzPO shows the both structured and structureless broad strong intramolecular charge transfer (ICT) absorption band at 334 nm, 348 nm and 377 nm (broad). Emission spectra are broad and structureless in solution except hexane, and the emission spectra are bathochromically shifted in polar solvents, both indications of an emission from an ICT state. The emission maximum changes from 375 nm in hexane to 484 nm in MeCN for 3CzPyPO and 382 nm in hexane to 502 nm in MeCN for t3CzPyPO. However, emission spectra are broad and structureless for t3CzPzPO in all solvents and emission maximum changes from 466 nm in hexane to 585 nm in DCM.

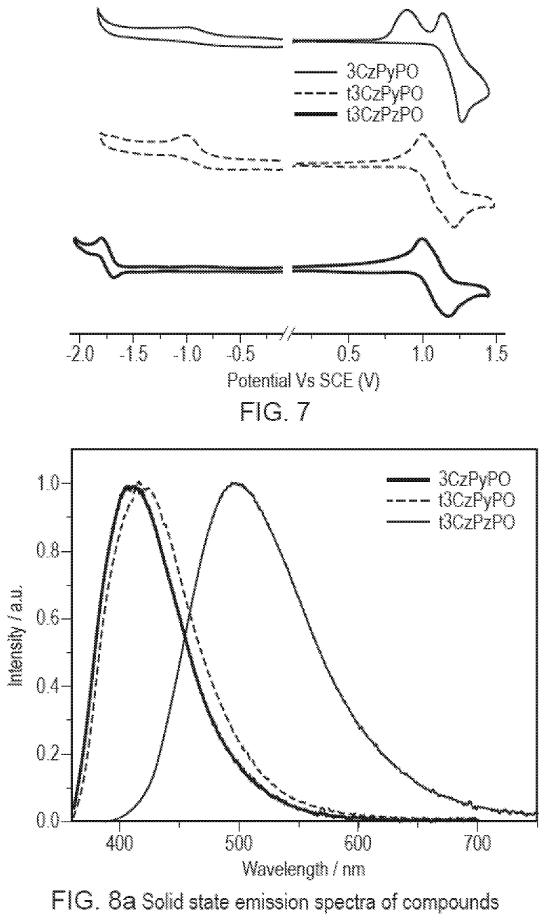

Electrochemical measurements on 3CzPyPO (XXXIII), t3CzPyPO (XXXIV) and t3CzPzPO (XXXVI) were carried out in dichloromethane. The cyclic voltammetry (CV) traces are shown in FIG. 7. The oxidation waves were found to be reversible for all three materials, while reduction waves were found to be irreversible for 3CzPyPO, t3CzPyPO and reversible for t3CzPzPO. The oxidation potential for 3CzPyPO [E_{1/2}^{(ox)}=1.20 V], t3CzPyPO [E_{1/2}^{(ox)}=1.10 V] and t3CzPzPO [E_{1/2}^{(ox)}=1.09 V]. The HOMO levels −5.54 eV, 5.44 eV and −5.43 eV for 3CzPyPO, t3CzPyPO and t3CzPzPO respectively, were calculated from E_{1/2}^{(ox)} (E_{HOMO}=E_{1/2}^{(ox)}+4.8−0.46).

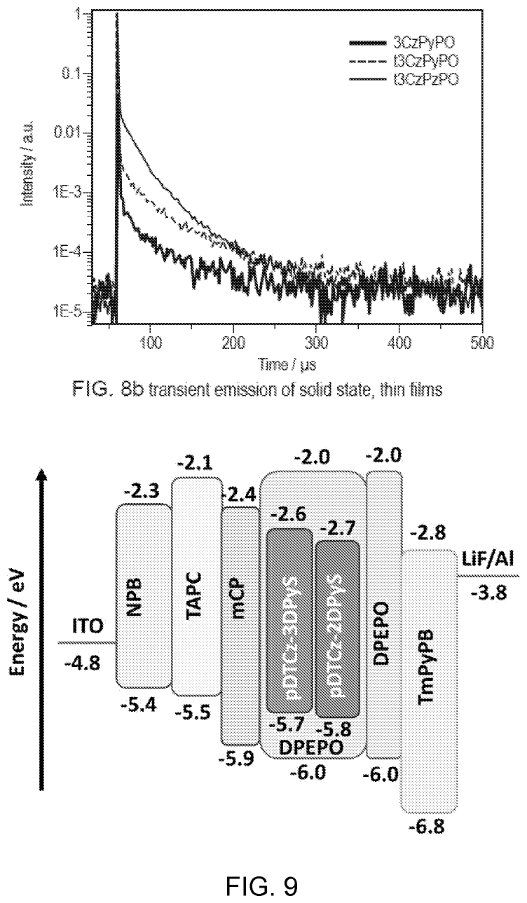

To study the photophysical properties in the thin-film state, these materials were co-doped with the host matrix (PMMA) in order to avoid concentration quenching. The solid state emission spectra are shown in FIG. 8a and the transient PL profile in FIG. 8b. The transient PL profile of 3CzPyPO (XXXIII) in PMMA at 300 K consists of fast and slow components with lifetimes of 9.2 ns and 29 μs ascribed to the prompt and delayed respectively. Similarly, lifetimes of 6.4 ns and 29 μs and 9.6 ns and 24.92 μs, respectively for t3CzPyPO (XXXIV) and t3CzPzPO (XXXVI) in PMMA film at 300 K. The PLQY measured in 10 wt % doped PMMA film under N₂ atmosphere of the doped thin films are 17%, 17.3% and 41.7%, respectively, for 3CzPyPO, t3CzPyPO and t3CzPzPO. The PLQY reduced to 15%, 15% and 35% for 3CzPyPO, t3CzPyPO and t3CzPzPO, respectively under air, indicating the presence of an accessible triplet state. This observation further confirms that these materials are TADF emitters.

Density Functional Theoretical (DFT) Calculations

To gain insight into structure-property relationships, density functional theoretical (DFT) calculations on pDTCz-2DPyS (also referred to as 2DPS-pDTCz), pDTCz-3DPyS (also referred to as 3DPS-pDTCz), p3Cz-3DPyS (also referred to as 3DPS-p3Cz) and pDTCz-3PPS (also referred to as 3PPS-pDTCz) (structures XV to XVIII) were carried out.

Initially the geometries of these emitters were fully optimized using a DFT methodology employing the PBE0 functional with the standard Pople 6-31G(d,p) basis set and Tamm-Dancoff approximation (TDA) was treated as a variant of Time-dependent density functional theory (TD-DFT). The molecular orbitals were visualized using GaussView 5.0 software. The four molecules exhibited similar HOMO and LUMO distribution with the HOMO are localized on the carbazole based donors and slightly extending to the heterocyclic rings, while the LUMO are localized on the sulfone and heterocyclic rings. There is partial overlap between HOMO and LUMO in the Het rings. This provides some explanation of the high oscillator strength of these four emitters.

The HOMO and LUMO energy level for pDTCz-2DPyS were calculated to be −5.77 eV and −1.71 eV, and the S, state and T₁ state were calculated to be 3.45 eV and 3.13 eV and ΔE_{ST} value is 0.32 eV. While the HOMO and LUMO energy level for pDTCz-3DPyS were calculated to be −5.74 eV and −1.51 eV, and the S, state and T₁ state were calculated to be 3.43 eV and 3.14 eV and ΔE_{ST} value is 0.29 eV. Although, pDTCz-2DPyS and pDTCz-3DPyS show comparable ΔE_{ST}, pDTCz-3DPyS exhibita higher a oscillator strength (Table 2). The high singlet energy of these molecules indicate that these materials are deep blue emitters and low calculated ΔE_{ST} values indicate that these materials are TADF emitters. Noticeably, the use of the extended donor (p3Cz-3DPyS) within the emitter design shows low ΔE_{ST} values. The calculated HOMO/LUMO energy level, singlet/triplet state and ΔE_{ST} are summarized in Table 2.

TABLE 2
Calculated HOMO/LUMO energy level and singlet/triplet state for
pDTCz-2DPyS (also referred to as 2DPS-pDTCz), pDTCz-3DPyS
(also referred to as 3DPS-pDTCz), p3Cz-3DPyS (also referred to
as 3DPS-p3Cz) and pDTCz-3PPS (also known as 3PPS-pDTCz).

| | HOMO | LUMO | | | ΔE_{ST} | Oscillator |
|---|---|---|---|---|---|---|
| Compounds | (eV) | (eV) | S₁(eV) | T₁(eV) | (eV) | strength (f) |
| pDTCz-2DPyS | −5.77 | −1.71 | 3.45 | 3.13 | 0.32 | 0.35 |
| pDTCz-3DPyS | −5.74 | −1.51 | 3.43 | 3.13 | 0.29 | 0.78 |
| pDTCz-3PPS | −5.76 | −1.55 | 3.26 | 2.78 | 0.48 | 0.34 |
| p3Cz-3DPyS | −5.60 | −2.18 | 3.00 | 2.84 | 0.16 | 0.36 |

TABLE 3
shows similarly calculated results for energy level and singlet/
triplet state for 3CzPyPO, t3CzPyPO, tCzPzPO, and t3CzPzPO.
(XXXIII, XXXIV, XXXV and XXXVI)

| | HOMO | LUMO | | | ΔE_{ST} | Oscillator |
|---|---|---|---|---|---|---|
| Compounds | (eV) | (eV) | S₁(eV) | T₁(eV) | (eV) | strength (f) |
| 3CzPyPO | −5.45 | −1.48 | 3.48 | 3.17 | 0.31 | 0.29 |
| t3CzPyPO | −5.24 | −1.15 | 3.37 | 3.10 | 0.27 | 0.27 |
| tCzPzPO | −5.75 | −1.53 | 3.50 | 2.92 | 0.58 | 0.24 |
| t3CzPzPO | −5.32 | −1.89 | 2.96 | 2.73 | 0.23 | 0.15 |

Testing of a Compound of the Invention in an OLED Device

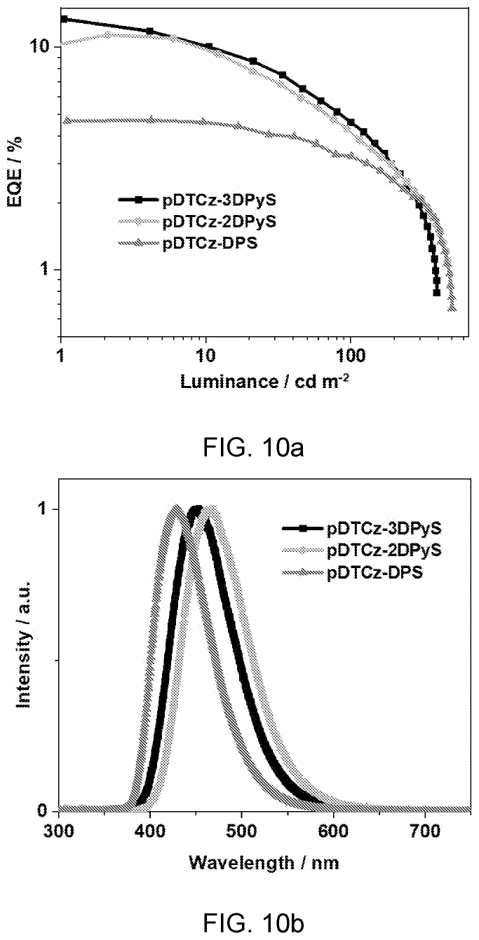

Multilayer devices were fabricated employing compounds XXVIII (pDTCz-3DPyS, also referred to as 3DPS-pDTCz), XXIX (pDTCz-2DPyS, also referred to as 2DPS-pDTCz) or reference compound (DPS-pDTCz) as emitters. FIG. 9 shows, in schematic form, the layers of an OLED device making use of compound XXVIII (pDTCz-3DPyS) and XXIX (pDTCz-2DPyS). FIG. 10 shows graphically the electroluminescent properties of the devices—the EQE (External Quantum Efficiency) and luminance, the electroluminescent spectra and device photos.

The electroluminescence (EL) properties of the pDTCz-3DPyS compounds were investigated using the following device structure: ITO/NPB (30 nm)/TAPC (20 nm)/mCP (10 nm)/DPEPO: pDTCz-3DPyS
or pDTCz-2DPyS or DPS-pDTCz (7 wt %) (30 nm)/PPT (5 nm)/TmPyPb (30 nm)/LiF (1 nm)/AI (100 nm). or pDTCz-2DPyS or pDTCz-DPS (7 wt %) (30 nm)/PPT (5 nm)/TmPyPb (30 nm)/LiF (1 nm)/AI (100 nm). pDTCz-3DPyS The N,N′-bis(1-naphthyl)-N,N′-diphenyl-1,1′-biphenyl-4,4′-diamine (NPB) layer on the ITO (indium tin oxide) anode layer acts as a hole injection material.

The 1,1-bis[4-[N,N′-di(p-tolyl)amino]phenyl]cyclohexane (TAPC) acts as a hole-transporting material.

1,3-Bis(N-carbazolyl)benzene (mCP) acts as an exciton blocker.

The (oxybis(2,1-phenylene))-bis(diphenylphosphine oxide) (DPEPO) acts an exciton blocker.

The 2,8-Bis(diphenylphosphoryl)dibenzo[b,d]thiophene (PPT) acts as an exciton blocking layer.

The 1,3,5-tri(m-pyrid-3-yl-phenyl)benzene (TmPyPb) is the electron-transporting material.

LiF and Al are used as the electron injection layer and the cathode, respectively.

The maximum external quantum efficiency (EQE) of 13.4%, 11.4% and 4.6, respectively for XXVII, XXIX and the reference compound were achieved. The devices comprising the compounds of the invention XXVIII and XXIX showed improved performance in EQE, current efficiency (CE) and power efficiency (PE) when compared with the device comprising the reference compound (see Table 4). The electroluminescence (EL) spectrum of the OLED containing XXVIII (pDTCz-3DPyS) and XXIX (pDTCz-2DPyS) exhibits blue emission with the emission maximum at 452 nm and 466 nm; the CIE coordinates of (0.15, 0.13) and (0.15; 0.18), respectively.

TABLE 4
The Electroluminescence performances of the OLEDs using
compounds XXVIII (pDTCz-3DPyS, also referred to as 3DPS-
pDTCz), XXIX (pDTCz-2DPyS, also referred to as 2DPS-pDTCz)
or reference compound (DPS-pDTCz)

| | | CEₘₐₓ/ | PEₘₐₓ/ | | |
|---|---|---|---|---|---|
| | EQEₘₐₓ/ | CE₁₀₀ | PE₁₀₀ | | |
| Devices | EQE₁₀₀ | (cd/A) | (lm/W) | λ_{EL}/nm | CIE @6 V |
| (pDTCz-3DPyS) | 13.4/4.5 | 13.2/4.3 | 10.9/2.3 | 452 | (0.15, 0.13) |
| (pDTCz-2DPyS) | 11.4/4.2 | 15.1/5.8 | 11.6/2.6 | 466 | (0.15, 0.18) |
| (pDTCz-DPS) | 4.6/3.2 | 2.5/1.7 | 2.2/1.0 | 428 | (0.15, 0.08) |

EQE, external quantum efficiency; CE, current efficiency; PE, power efficiency; data are reported as maxima and at 100 cd m⁻²; and λ_{EL}, the wavelength where the EL spectrum has the highest intensity, CIE=Internationale de L'Éclairage coordinates. The electroluminescence (EL) properties of the compounds XXXII and XXXIIa were investigated using the following device structure: ITO/TAPC (40 nm)/mCP (10 nm)/DPEPO: pDTCz-DPmS or pDTCz-DPzS (7 wt %) (30 nm)/PPT (5 nm)/TmPyPb (30 nm)/LiF (1 nm)/AI (100 nm).

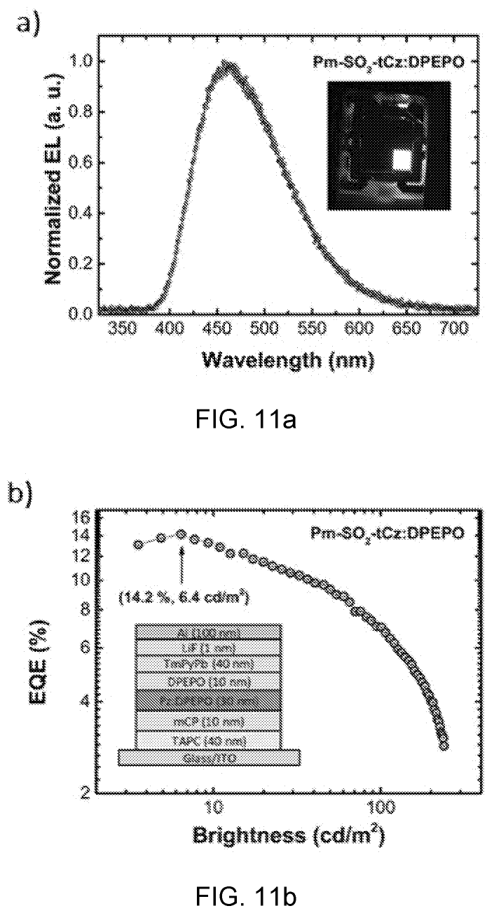

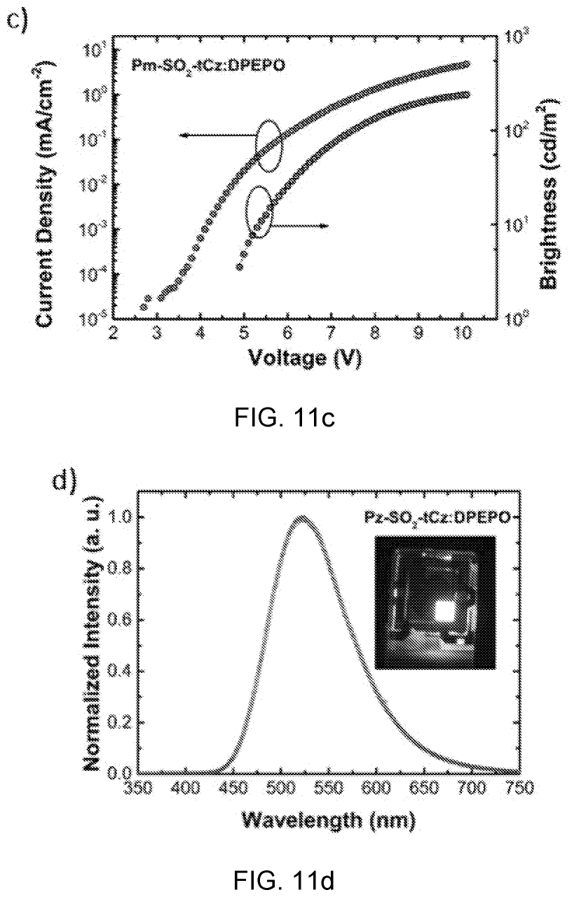

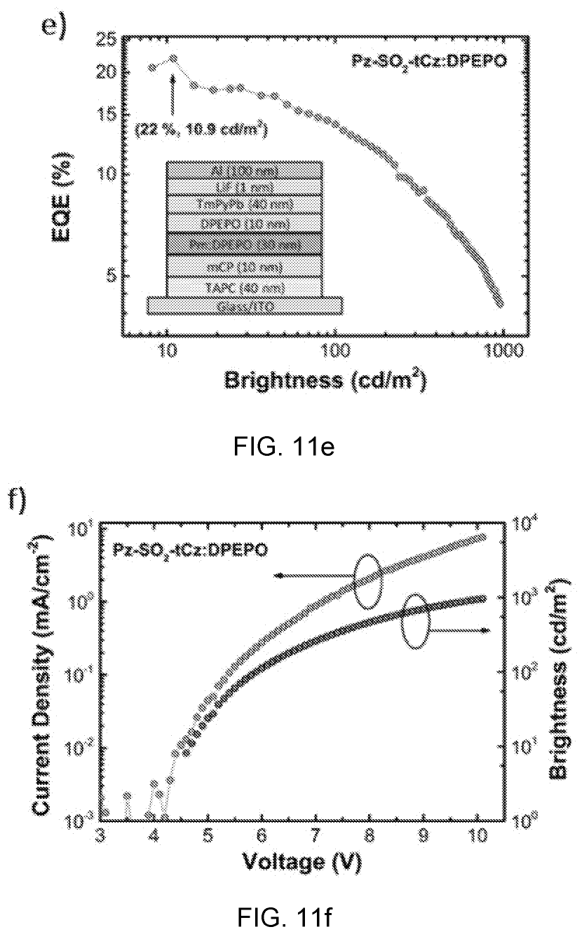

Plots in FIGS. 11b and 11e show EQE as a function of brightness. A maximum EQE of 22% and 14% respectively for XXXII and XXXIIa were achieved. The devices comprising the compounds of the invention XXXII and XXXIIa showed improved EQE (see Table 5). The electroluminescence (EL) spectrum of the OLED containing XXXII (pDTCz-DPzS) and XXXIIa (pDTCz-DPmS) exhibits green and blue emission, respectively with the emission maximum at 522 nm and 461 nm; the CIE coordinates of (0.31; 0.53) and (0.19; 0.26), respectively (see FIGS. 11a and 11d).

Plots in FIGS. 11c and 11f show the current density versus voltage and brightness versus voltage curves of devices containing pDTCz-DPmS (also referred to as Pm—SO₂-tCz or XXXIIa) and pDTCz-PzS (also referred to as pz-SO₂-tCz or XXXII). The devices exhibit low turn-on voltages (˜3.6 V for that containing pDTCz-DPmS and ˜4.4 V for that containing pDTCz-DPzS) and high brightness levels (reaching 240 cd/m² for that containing pDTCz-DPmS and 961 cd/m² for that containing pDTCz-DPzS).

TABLE 5
The Electroluminescence performances of the OLEDs using
compounds pDTCz-DPmS and pDTCz-DPzS as the emitters.

| | EQEₘₐₓ/ | Brtₘₐₓ | EQE₁₀₀/ | CIE | λ_{EL}/ |
|---|---|---|---|---|---|
| Device | % | cd/m² | % | (x, y) | nm |
| pDTCz-DPmS | 14 | 240 | 7 | (0.19, 0.26) | 461 |
| pDTCz-DPzS | 22 | 961 | 14 | (0.31, 0.53) | 522 |

EQE = External quantum efficiency;
Brt = brightness.
Subscript 100 refers to values taken at 100 cd/m².
CIE = Internationale de L'Éclairage coordinates.

1-43. (canceled)

44. An organic thermally activated delayed fluorescence (TADF) compound selected from the group consisting of compounds according to formula Ia and formula Ib: 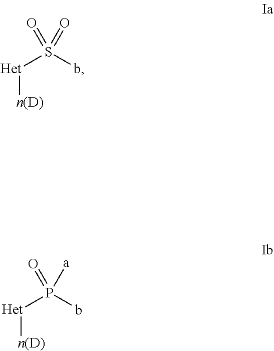
wherein:
Het is a heteroaryl group containing at least one heteroatom;
n(D)denotes n donor groups D bonding to the heteroaryl group Het;
n is at least 1; and
a and -b denotes bonding to another group.

45. The organic TADF compound of claim 44 wherein each group Het is, independently for each occurrence, selected from the group consisting of substituted and unsubstituted pyridyl, pyridazinyl, pyrazinyl, pyrimidinyl, 1,2,4-triazinyl, 1,3,5-triazinyl, imidazolyl benzimidazolyl indolyl, quinolinyl, benzothiazolyl, purinyl thiophenyl, benzothiophenyl, oxadiazolyl, benzoxadiazolyl, thiazolyl, quinazolinyl, phthalazinyl and pteridinyl.

46. The organic TADF compound of claim 44 wherein for the TADF compound according to formula Ia, each group Het is not 2-pyridyl.

47. The organic TADF compound of claim 44 wherein each group Het is not 2-pyridyl.

48. The organic TADF compound of claim 44, wherein the group bonded at position -b for compounds of formula Ia or formula Ib is selected from the group consisting of: —H, substituted or unsubstituted primary, secondary or tertiary alkyl, that may be cyclic and may be unsaturated; substituted or unsubstituted aryl or heteroaryl; substituted or unsubstituted aryl hydroxyl; substituted or unsubstituted aryloxy; and substituted or unsubstituted thioalkyl or thioaryl.

49. The organic TADF compound of claim 48 wherein the group bonded at position -b is an aryl or heteroaryl group.

50. The organic TADF compound of claim 48 wherein the group bonded at position -b is an aryl or heteroaryl group comprising at least one donor group D as a substituent.

51. The organic TADF compound of claim 44, and according to formula Ib, wherein the group at position -a is selected from the group consisting of: —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; substituted or unsubstituted aryloxy or heteroaryloxy; substituted or unsubstituted aryl hydroxyl; substituted or unsubstituted aryloxy; and substituted or unsubstituted thioalkyl or thioaryl.

52. The organic TADF compound of claim 44, wherein the group bonding at position -a has oxygen bonding to the phosphorus.

53. The organic TADF compound of claim 51 wherein the group bonding at position -a is aryl or heteroaryl and is optionally substituted with one or more donor groups D.

54. The organic TADF compound of claim 44, selected from the group consisting of compounds according to formula IIIa and formula IIIb 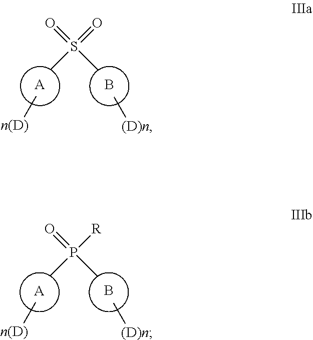
wherein:
rings A and B are aryl and at least one ring is a heteroaryl group Het;
n(D) denotes, independently for each occurrence, n donor groups D bonding to the respective rings A and B;
n is at least 1 for the at least one group Het; and
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; substituted or unsubstituted aryloxy, heteroaryloxy; substituted or unsubstituted aryl hydroxyl; substituted or unsubstituted aryloxy; and substituted or unsubstituted thioalkyl or thioaryl.

55. The organic TADF compound of claim 44, according to formula IIIc: 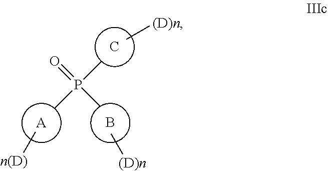
wherein:
rings A, B and C are aryl and at least one ring is a heteroaryl group Het;
n(D) denotes, independently for each occurrence, n donor groups D bonding to the respective rings A, B and C; and
n is at least 1 for the at least one group Het.

56. The organic TADF compound of claim 44, selected from the group consisting of compounds according to formulas IIId, IIIe and IIIf: 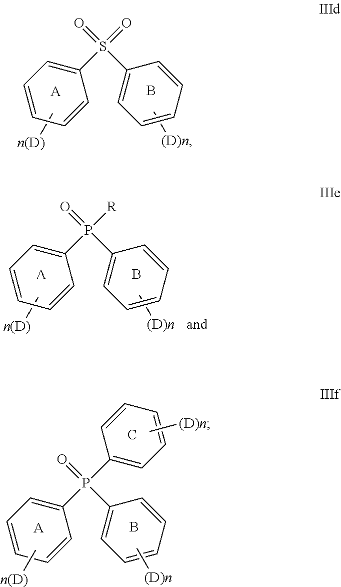
wherein at least one of the positions in one of the six membered rings A, B and C is a nitrogen atom;
n(D)- denotes the presence of n donor groups D each bonded to a carbon atom in the respective ring; and
wherein n is at least 1 for one of the rings that contains a nitrogen atom; and
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; and substituted or unsubstituted aryloxy or heteroaryloxy; substituted or unsubstituted aryl hydroxyl; and substituted or unsubstituted thioalkyl or thioaryl.

57. The organic TADF compound of claim 44 selected from the group consisting of compounds according to formula IVa and formula IVb: 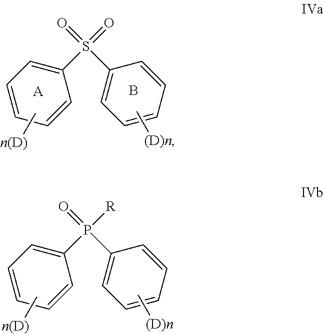
wherein at least one of the positions in one of the six membered rings is a heteroatom;
n(D)-denotes the presence of n donor groups D each bonded to a carbon atom in the respective ring; wherein n is at least 1 for one of the rings; and
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; and substituted or unsubstituted aryloxy or heteroaryloxy; substituted or unsubstituted aryl hydroxyl; and substituted or unsubstituted thioalkyl or thioaryl.

58. The organic TADF compound of claim 44, wherein carbon atoms that are not bonded to a donor group D or to the acceptor moieties 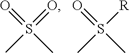
are independently for each occurrence selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo, aryl, aryl hydroxy, amino, alkoxy, alkylthio, carboxy, cyano, thio, formyl, ester, acyl, thioacyl, amido, sulfonamido, and carbamate.

59. The organic TADF compound of claim 44 selected from the group consisting of: 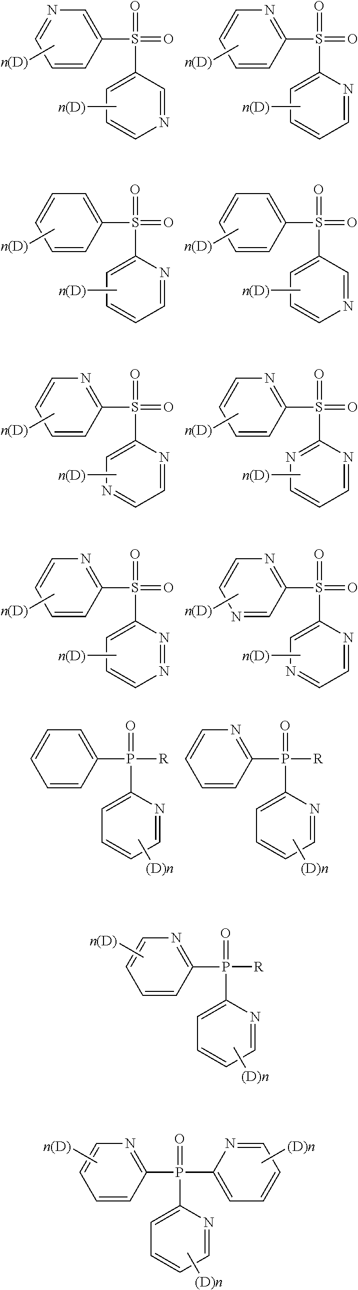 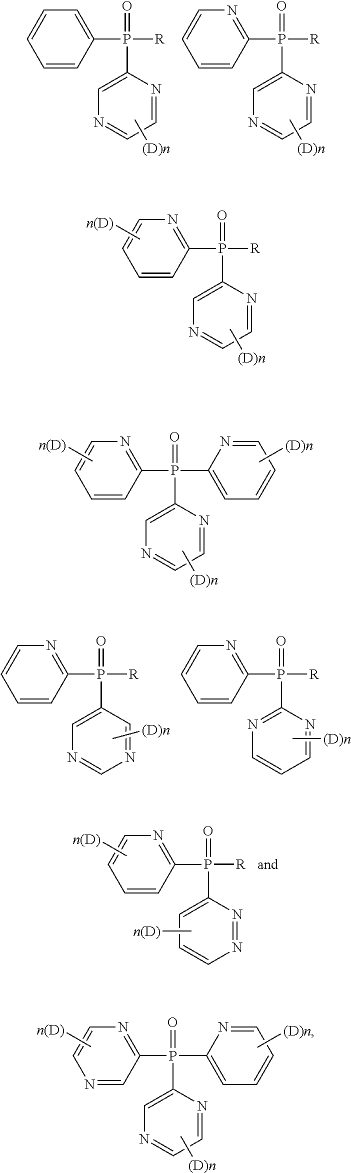
wherein:
n is from 0 to 5;
n is at least 1 for one heteroaryl ring in the structure; and
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; and substituted or unsubstituted aryloxy or heteroaryloxy substituted or unsubstituted aryl hydroxyl; and substituted or unsubstituted thioalkyl or thioaryl.

60. The organic TADF compound of claim 44 selected from the group consisting of:
compounds according to formula Va, Vb, VIa and VIb: 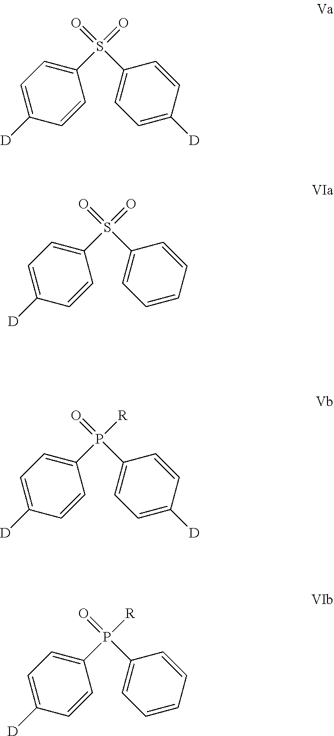
wherein D are donor groups;
wherein at least one of the positions in a six membered ring including a group D is a heteroatom; and
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; and substituted or unsubstituted aryloxy or heteroaryloxy; substituted or unsubstituted aryl hydroxyl; and substituted or unsubstituted thioalkyl or thioaryl.

61. The organic TADF compound of claim 44 selected from the group consisting of:

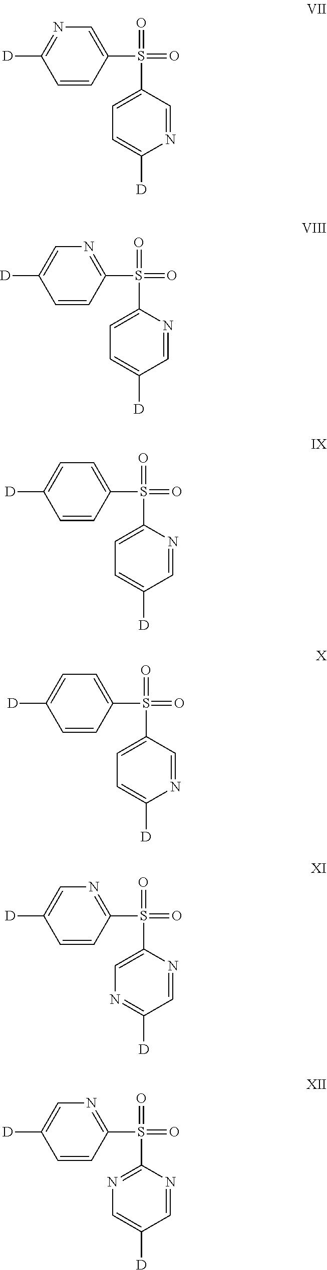

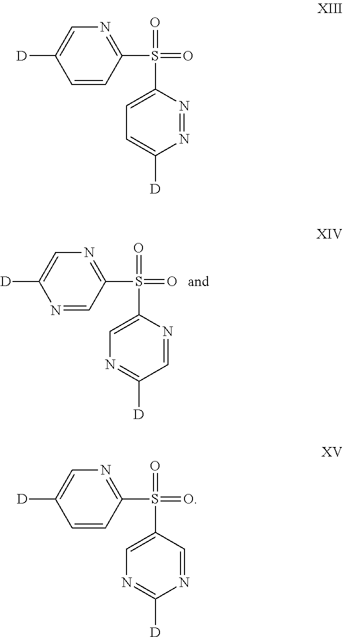 [SMILES: [2H]c1ccc(S(=O)(=O)c2ccc([2H])nn2)nc1 ; [2H]c1cnc(S(=O)(=O)c2cnc([2H])cn2)cn1 ; "and" ; [2H]c1ccc(S(=O)(=O)c2cnc([2H])nc2)nc1]

62. The organic TADF compound of claim 44 selected from the group consisting of:

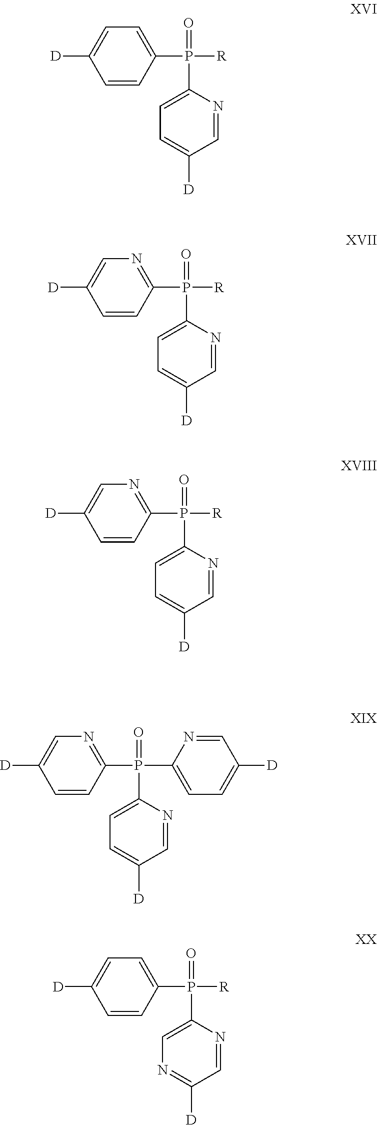

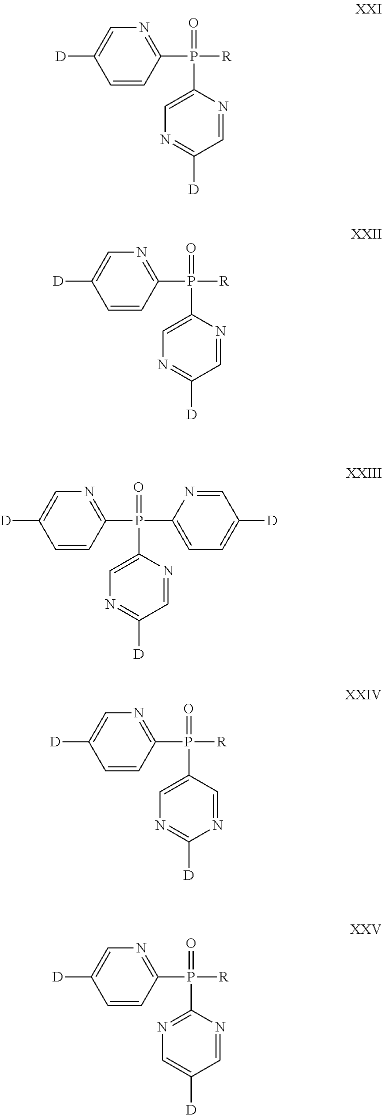

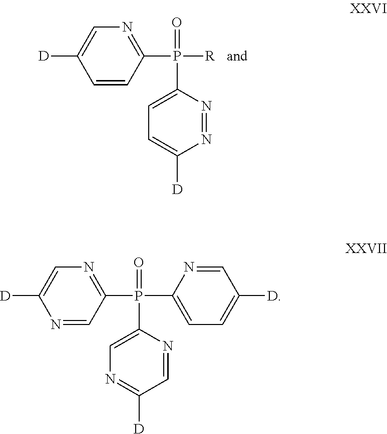

63. The organic TADF compound of claim 44, wherein the compound has two heteroaryl groups Het bonded to S or P, and wherein each Het group may be the same or different.

64. The organic TADF compound of claim 54, wherein the compound has two heteroaryl groups Het bonded to S, and wherein each Het group may be the same or different.

65. The organic TADF compound of claim 54, wherein R is selected from the group consisting of substituted or unsubstituted primary, secondary or tertiary alkoxy, that may be cyclic and may be unsaturated; substituted or unsubstituted aryloxy, heteroaryloxy; substituted or unsubstituted aryl hydroxyl; and substituted or unsubstituted aryloxy.

66. The organic TADF compound of claim 44, wherein donor groups D are selected from the group consisting of: 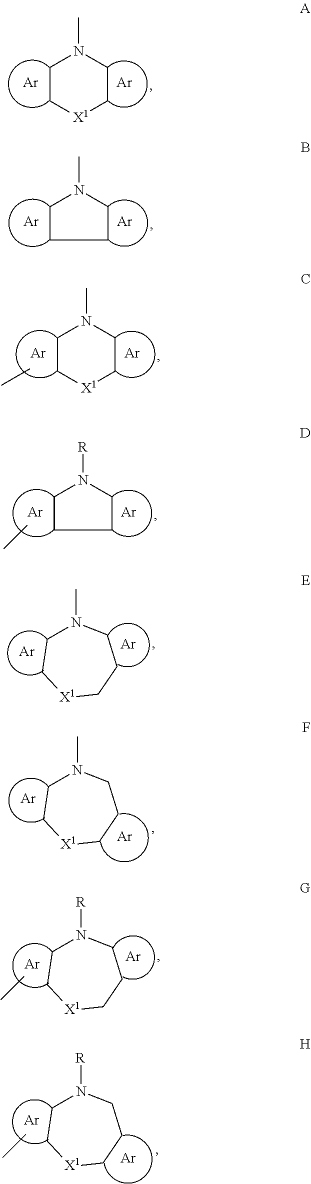 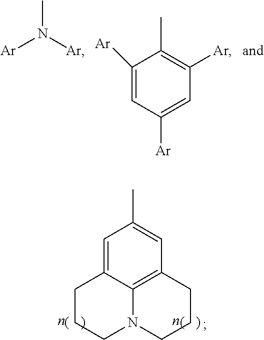
wherein X¹ is selected from the group consisting of O, S, NR, SiR₂, PR and CR₂, wherein each R is independently selected from the group consisting of —H, alkyl, aryl or heteroaryl;
each Ar is independently for each occurrence selected from the group consisting of substituted or unsubstituted aryl or heteroaryl; and 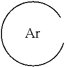
represents, independently for each occurrence a substituted or unsubstituted aryl or heteroaryl ring fused to the central ring of structures A B, C, D, E or F, selected from a five or a six membered substituted or unsubstituted aryl or heteroaryl ring, and in structures C, D, G and H bonding to the rest of the molecule is para to the nitrogen;
n ( ) indicates the optional presence of saturated —CH₂— groups in the rings annelated to the benzene ring, wherein n is independently for each occurrence, 0, 1, or 2; substituents on —Ar and 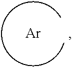
where present can include phosphine oxide or phosphine sulphide, to moderate the donor properties.

67. The organic TADF compound of claim 44, wherein donor groups D are selected from the group consisting of: 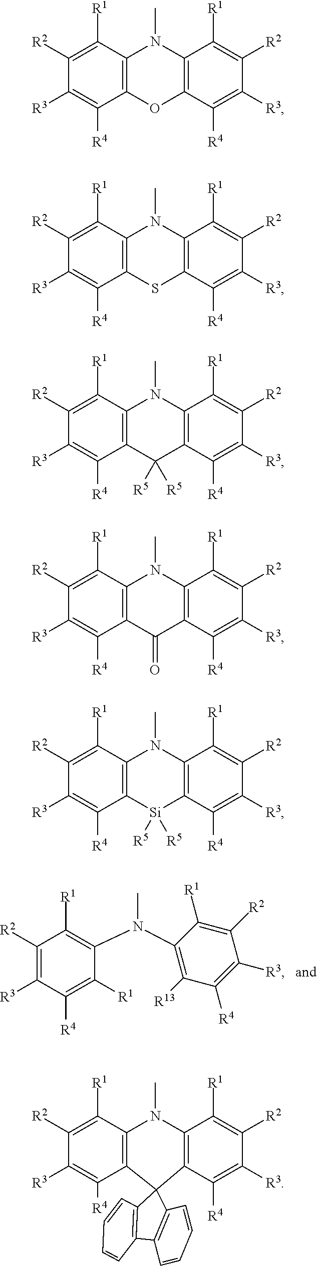
wherein each group R¹, R², R³, R⁴, and R⁵ is, independently for each occurrence, selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo, aryl, aryl hydroxy, amino, alkoxy, alkylthio, carboxy, cyano, thio, formyl, ester, acyl, thioacyl, amido, sulfonamido, carbamate, phosphine oxide, and phosphine sulphide; and
wherein in the structure 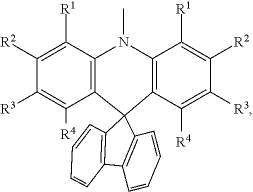
the fluorene moiety may have one or more of the hydrogens substituted by the options indicated for the groups R¹, R², R³, R⁴ and R⁵.

68. The organic TADF compound of claim 44, wherein donor groups D are selected from the group consisting of: 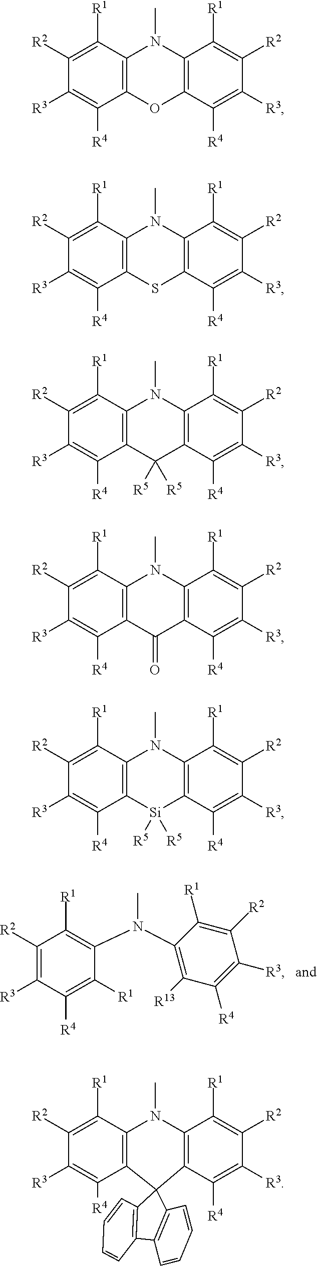
wherein each group R¹, R², R³, R⁴, and R⁵ is, independently for each occurrence, selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo (e.g. fluoro, chloro, bromo and iodo), aryl, aryl hydroxy, amino, alkoxy, alkylthio, carboxy, cyano, thio, formyl, ester, acyl, thioacyl, amido, sulfonamido, carbamate, phosphine oxide, and phosphine sulphide; and
wherein in the structure 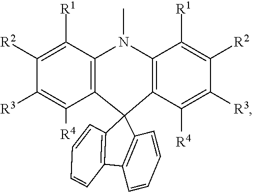
the fluorene moiety may have one or more of the hydrogens substituted by the options indicated for the groups R¹, R², R³, R⁴ and R⁵.

69. The organic TADF compound of claim 44, wherein when the TADF compound is selected from the group consisting of compounds according to formula Ia, donor groups D are not:

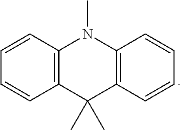 [SMILES: CN1c2ccccc2C(C)(C)c2ccccc21 ; "."]

70. The organic TADF compound of claim 44, wherein donor groups D are selected from the group consisting of: 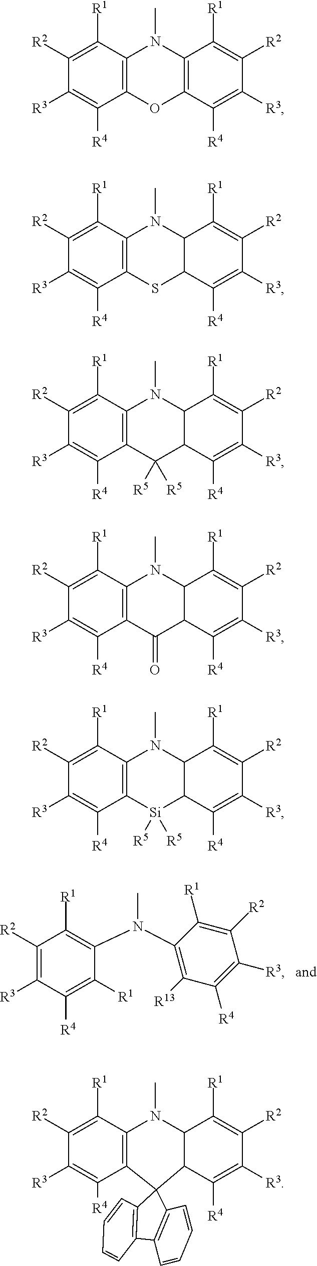
wherein each group R¹, R², R³, R⁴, and R⁵ is, independently for each occurrence, selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo, aryl, aryl hydroxy, amino, alkoxy, alkylthio, carboxy, cyano, thio, formyl, ester, acyl, thioacyl, amido, sulfonamido, carbamate, phosphine oxide, and phosphine sulphide; and
wherein in the structure 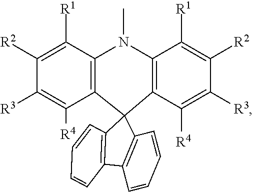
the fluorene moiety may have one or more of the hydrogens substituted by the options indicated for the groups R¹, R², R³, R⁴ and R⁵.

71. The organic TADF compound of claim 44, wherein donor groups D are selected from the group consisting of: 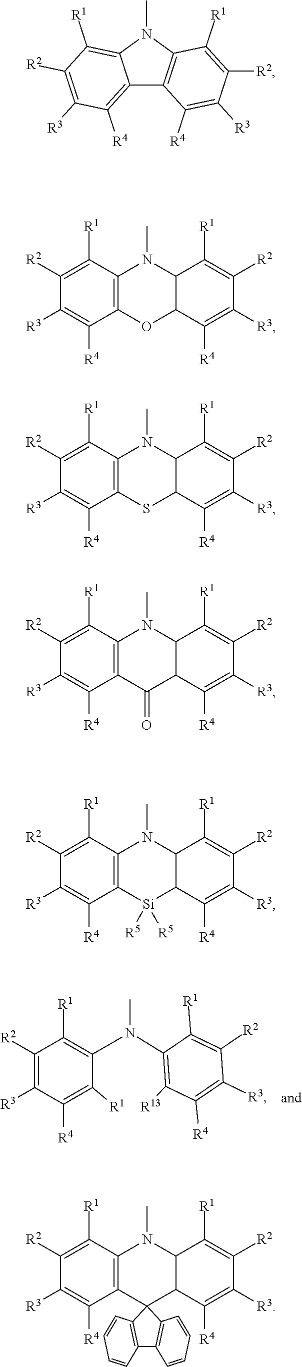
wherein each group R¹, R², R³, R⁴, and R⁵ is, independently for each occurrence, selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo, aryl, aryl hydroxy, amino, alkoxy, alkylthio, carboxy, cyano, thio, formyl, ester, acyl, thioacyl, amido, sulfonamido, carbamate, phosphine oxide, and phosphine sulphide; and
wherein in the structure 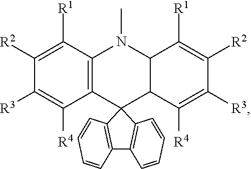
the fluorene moiety may have one or more of the hydrogens substituted by the options indicated for the groups R¹, R², R³, R⁴ and R⁵.

72. The organic TADF compound of claim 44 wherein donor groups D are of the form; 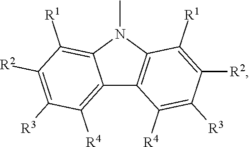
wherein each group R¹, R², R³ and R⁴ is, independently for each occurrence, selected from the group consisting of —H, substituted or unsubstituted primary, secondary or tertiary alkyl, that may be cyclic and may be unsaturated; substituted or unsubstituted aryl or heteroaryl, —CF₃, —OMe, —SF₅, —NO₂, halo, aryl, aryl hydroxy, amino, alkoxy, alkylthio, carboxy, cyano, thio, formyl, ester, acyl, thioacyl, amido, sulfonamido, carbamate, phosphine oxide and phosphine sulphide.

73. The organic TADF compound of claim 72, wherein at least one donor group D is selected from the group consisting of:

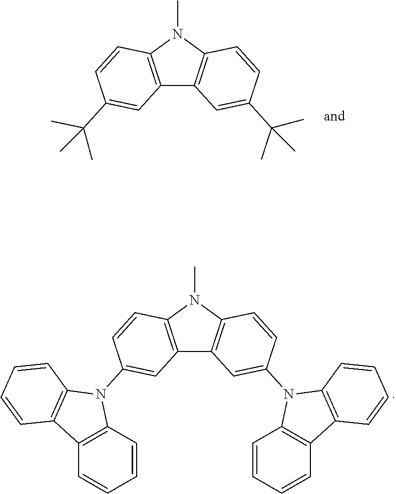 [SMILES: Cn1c2ccc(C(C)(C)C)cc2c2cc(C(C)(C)C)ccc21 ; Cn1c2ccc(-n3c4ccccc4c4ccccc43)cc2c2cc(-n3c4ccccc4c4ccccc43)ccc21]

74. The organic TADF compound of claim 44, wherein the compound is not:

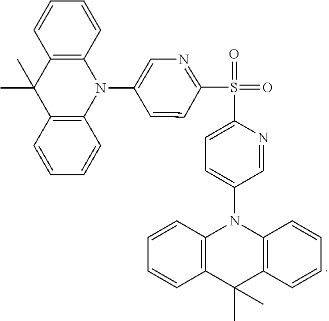 [SMILES: CC1(C)c2ccccc2N(c2ccc(S(=O)(=O)c3ccc(N4c5ccccc5C(C)(C)c5ccccc54)cn3)nc2)c2ccccc21]

75. The organic TADF compound of claim 44, wherein the compound is not a sulphone-containing compound of formula Ia which contains a donor group D which is:

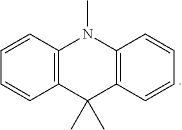 [SMILES: CN1c2ccccc2C(C)(C)c2ccccc21]

76. The organic TADF compound of claim 44, wherein the compound is not a sulphone-containing compound of formula Ia which contains two heteroaryl group Hets bonded to the accepting moiety wherein each Het group is 2-pyridyl or wherein each Het group is pyridyl.

77. The organic TADF compound of claim 44, wherein the compound is a sulphone-containing compound of formula Ia which contains two heteroaryl group Hets and wherein the compound is not 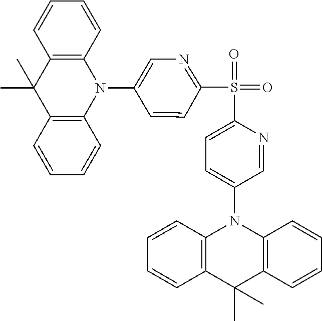 [SMILES: CC1(C)c2ccccc2N(c2ccc(S(=O)(=O)c3ccc(N4c5ccccc5C(C)(C)c5ccccc54)cn3)nc2)c2ccccc21]
or
wherein the compound does not contain a donor group D which is: 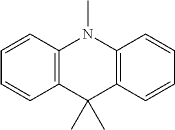 [SMILES: CN1c2ccccc2C(C)(C)c2ccccc21]
or
wherein both Het groups are not 2-pyridyl or not pyridyl.

78. The organic TADF compound of claim 44, wherein when the TADF compound is not

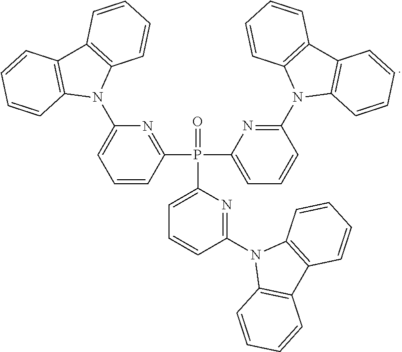 [SMILES: O=P(c1cccc(-n2c3ccccc3c3ccccc32)n1)(c1cccc(-n2c3ccccc3c3ccccc32)n1)c1cccc(-n2c3ccccc3c3ccccc32)n1]

79. The organic TADF compound of claim 44, wherein the compound is not: 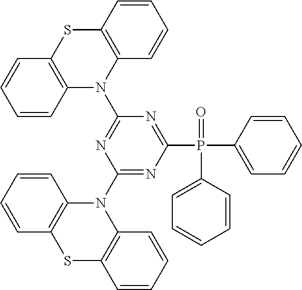 [SMILES: O=P(c1ccccc1)(c1ccccc1)c1nc(N2c3ccccc3Sc3ccccc32)nc(N2c3ccccc3Sc3ccccc32)n1]
or wherein the TADF compound is not a phosphorus containing compound of formula Ib which contains a donor group D which is:

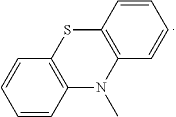

80. The organic TADF compound of claim 44, wherein the compound is not a phosphorus containing compound of formula Ib which contains a donor group D which is: 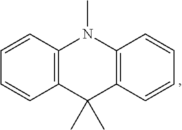 [SMILES: CN1c2ccccc2C(C)(C)c2ccccc21]
substituted or unsubstituted.

81. The organic TADF compound of claim 44, wherein the compound is a phosphorus containing compound of formula Ib or of formula IIIb, IVb, IIIe, Vb or VIb, 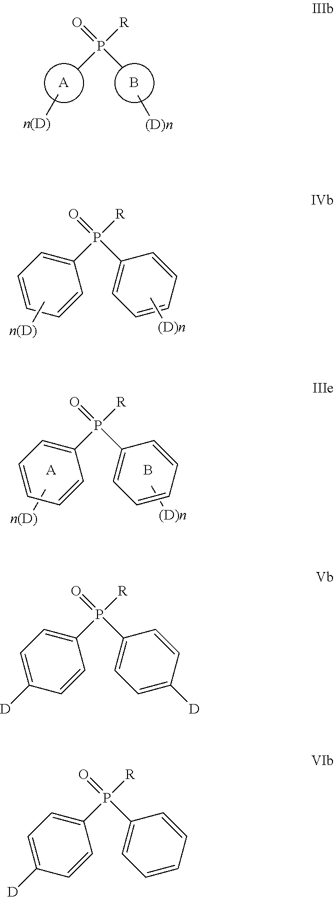
wherein at least one of the positions in one of the six membered rings A and B is a nitrogen atom;
n(D)-denotes the presence of n donor groups D each bonded to a carbon atom in the respective ring; wherein D are donor groups;
wherein at least one of the positions in a six membered ring including a group D is a heteroatom;
R is selected from the group consisting of —H, substituted or unsubstituted primary, secondary or tertiary alkyl, that may be cyclic and may be unsaturated; substituted or unsubstituted aryl or heteroaryl; substituted or unsubstituted primary, secondary or tertiary alkoxy, that may be cyclic and may be unsaturated; and substituted or unsubstituted aryloxy or heteroaryloxy; substituted or unsubstituted aryl hydroxyl; and substituted or unsubstituted thioalkyl or thioaryl; and
wherein the group at position a of formula Ib or R of formula IIIb, IVb, IIIe, Vb or VIb is bonded to the phosphorus atom via a heteroatom.

82. The organic TADF compound of claim 81, wherein said compound is a phosphinate or a phosphonate.

83. The organic TADF compound of claim 44 selected from the group consisting of:

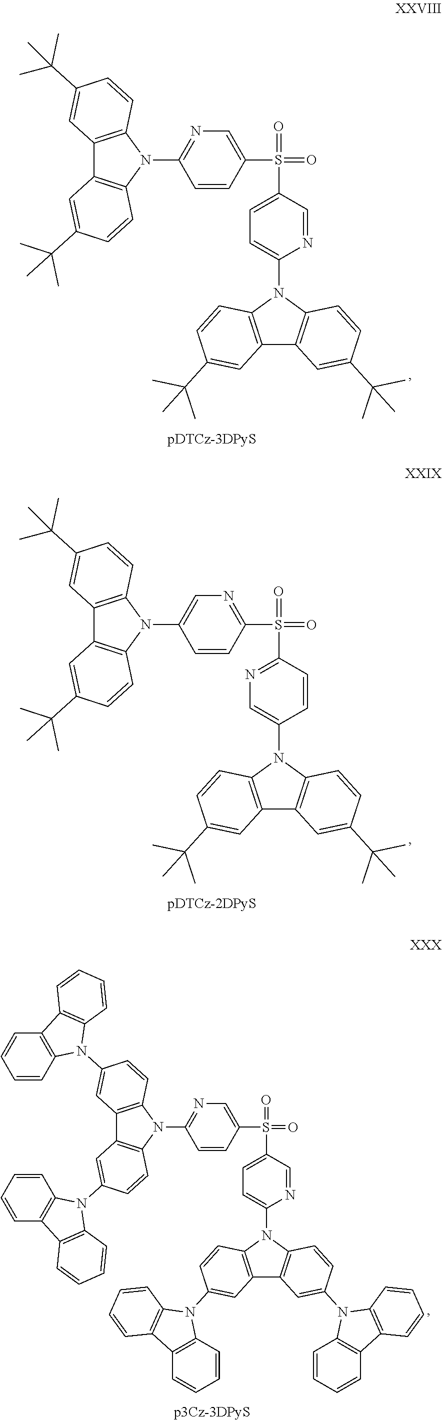

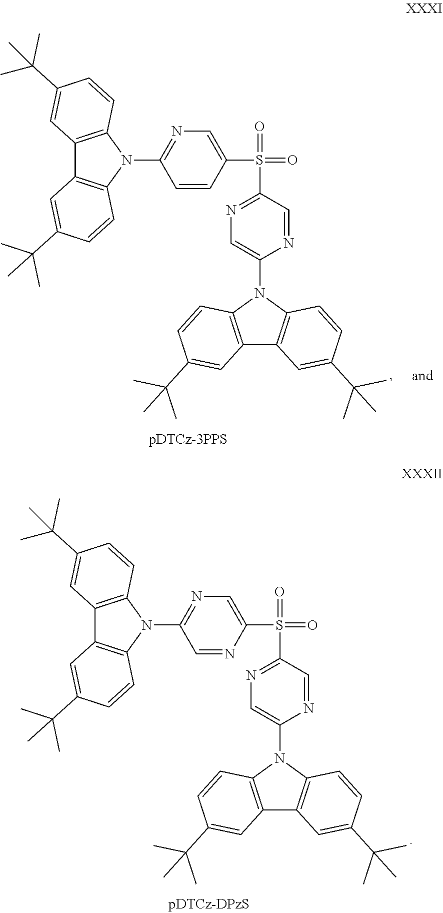

84. The organic TADF compound of claim 44 selected from the group consisting of:

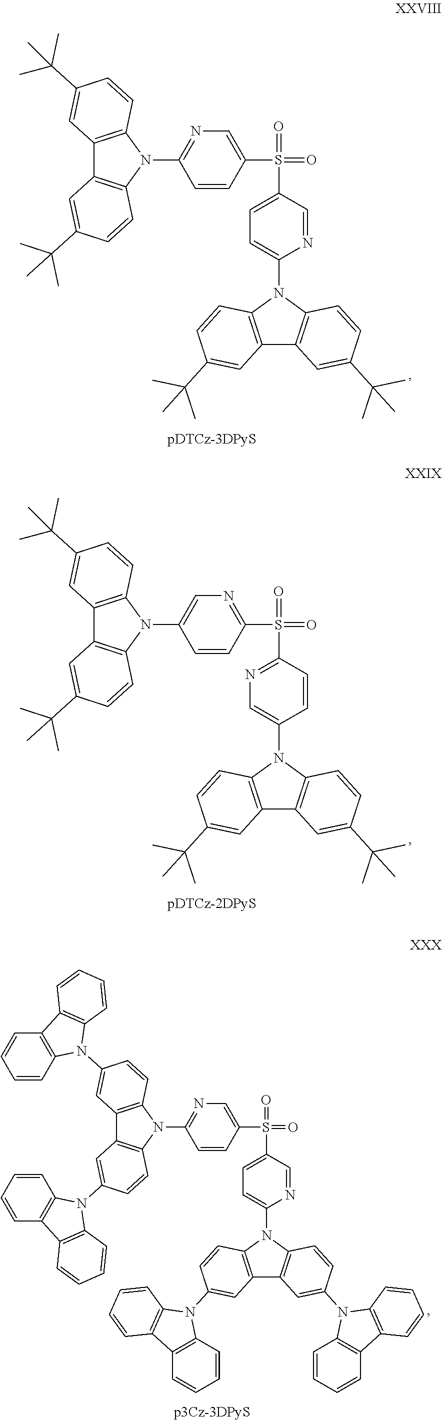

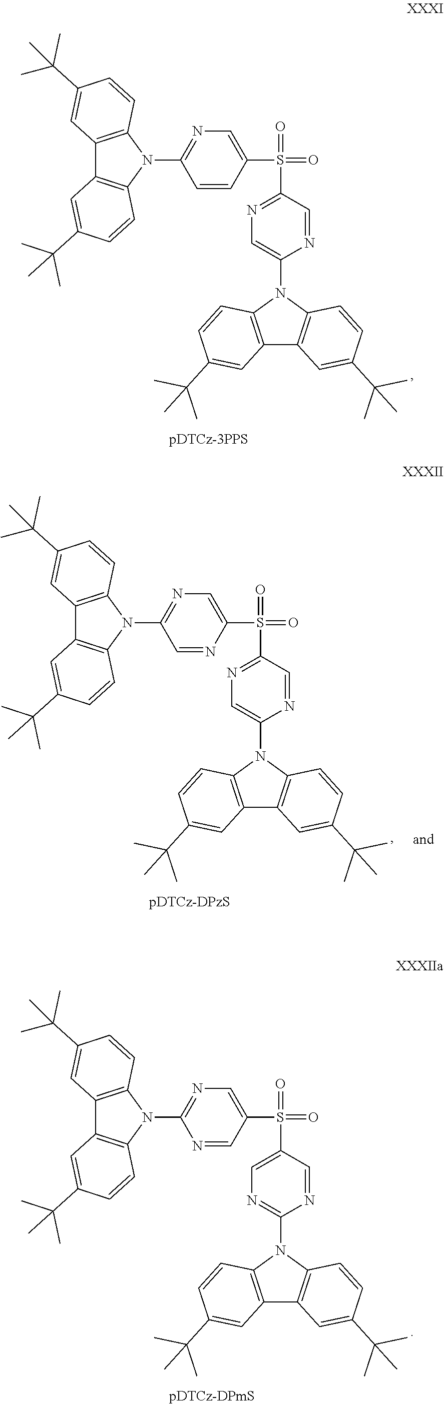

85. The organic TADF compound of claim 44 selected from the group consisting of:

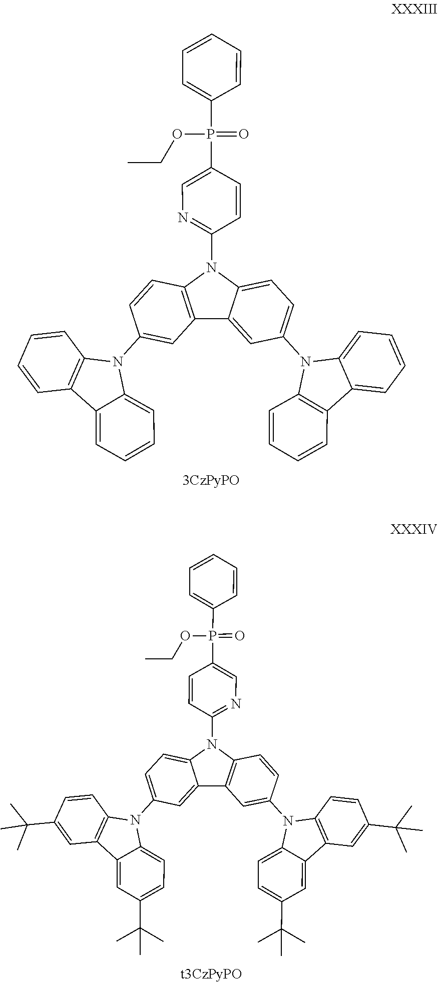 [SMILES: CCOP(=O)(c1ccccc1)c1ccc(-n2c3ccc(-n4c5ccccc5c5ccccc54)cc3c3cc(-n4c5ccccc5c5ccccc54)ccc32)nc1 ; CCOP(=O)(c1ccccc1)c1ccc(-n2c3ccc(-n4c5ccc(C(C)(C)C)cc5c5cc(C(C)(C)C)ccc54)cc3c3cc(-n4c5ccc(C(C)(C)C)cc5c5cc(C(C)(C)C)ccc54)ccc32)nc1]

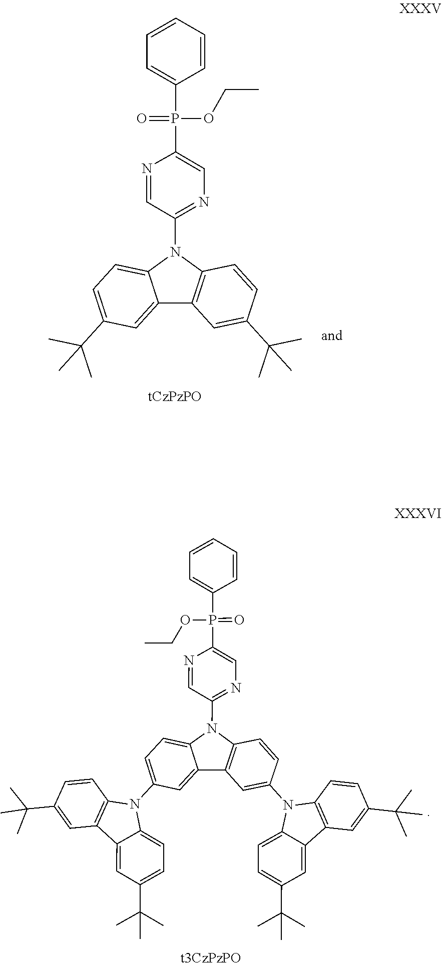 [SMILES: CCOP(=O)(c1ccccc1)c1cnc(-n2c3ccc(C(C)(C)C)cc3c3cc(C(C)(C)C)ccc32)cn1 ; CCOP(=O)(c1ccccc1)c1cnc(-n2c3ccc(-n4c5ccc(C(C)(C)C)cc5c5cc(C(C)(C)C)ccc54)cc3c3cc(-n4c5ccc(C(C)(C)C)cc5c5cc(C(C)(C)C)ccc54)ccc32)cn1]

86. An electroluminescent device such as an OLED or a light emitting electrochemical cell (LEEC) comprising one or more of the compounds according to claim 44, as an emitter material.
